(19) United States
(12) Patent Application Publication
(10) Pub. No.: US 2021/0155955 A1
(43) Pub. Date: May 27, 2021

(54) THERAPEUTIC CONSTRUCTS FOR TREATING CANCER

(71) Applicant: CANCER TARGETING SYSTEMS, INC., Newton Centre, MA (US)

(72) Inventors: Christopher Ullman, Hertfordshire (GB); Christine Anne Carrington, Cambridgeshire (GB)

(21) Appl. No.: 17/047,008

(22) PCT Filed: Apr. 10, 2019
(86) PCT No.: PCT/US2019/026822

§ 371 Date: Oct. 12, 2020

Related U.S. Application Data
(60) Provisional application No. 62/655,922, filed on Apr. 11, 2018.

(51) Int. Cl. C12N 15/86; C07K 14/54; C07K 16/28; C07K 14/195; A61K 9/51; A61P 35/00

(52) U.S. Cl. CPC C12N 15/86; C07K 14/5434; C07K 16/2818; C12Y 207/01021; C07K 14/195; A61K 9/5146; A61P 35/00; C07K 16/2827

(57) ABSTRACT

The present disclosure provides nucleic acid constructs for the treatment of cancer, comprising a cancer-specific promoter and one or more therapeutic genes.

### CROSS-REFERENCE TO RELATED APPLICATION

The present application claims priority to U.S. Provisional Patent Application No. 62/655,922, filed on Apr. 11, 2018, the contents of which are hereby incorporated by reference in their entirety.

### FIELD OF TECHNOLOGY

The present technology generally relates to genetic constructs and methods for their use in cancer treatment. In particular, transcription of genes in the constructs is driven by cancer specific promoters so that expression is directly within the tumor microenvironment.

### BACKGROUND

Targeted treatment of cancer, and especially metastases, remains an important but elusive goal. Systemic cancer treatments can cause toxicity by inappropriate activation of the immune system in healthy tissues. By precisely directing expression of anti-cancer agents within the cancer cells, higher concentrations of these agents can be achieved within the tumor and lower levels elsewhere. Cancer-cell specific/selective promoters, with broad activity across a wide range of different tumor cells, can be used to direct the expression of single or multiple anti-cancer agents to stimulate local activation of the immune system or release suppression through inhibition of immunological checkpoints.

While investigators use many strategies to provide tumor therapies, high systemic toxicity and non-specific activity limit their acceptance. One such agent is interleukin-12 (IL-12), which is known to have potent anti-tumor activities but has undesirable side effects when administered systemically, either subcutaneously or intravenously (Car, et al., 1999, Tox. Pathology 27, 58-63). Having the ability to limit expression to within the tumor microenvironment will enable therapeutic levels of IL-12 to be produced at the tumor site, where it is most needed therapeutically in diseased tissue, and not elsewhere in healthy tissues in the body.

U.S. Pat. No. 6,737,523 (Fisher, et al.), the complete contents of which is hereby incorporated by reference, describes a progression elevated gene-3 (PEG-3) promoter, which is specific for directing gene expression in cancer cells. The patent describes the use of the promoter to express genes of interest in cancer cells in a specific manner.

United States Patent Publication No. 2009/0311664 describes cancer cell detection using viral vectors that are conditionally competent for expression of a reporter gene only in cancer cells.

There is an ongoing need to develop improved methods of cancer treatment that can be administered systemically while being highly specific for cancer cells and enabling expression of therapeutic agents. Plasmid-based nanoparticles offer the opportunity to deliver such agents. Indeed, the CpG content of such plasmids has been shown to elicit immune activation that can assist an anti-cancer response (Bode et al., 2011, Expert Rev Vaccines 10, 499-511). Therefore, for cancer treatment, it has been perceived as a benefit not to reduce CpG content. However, in other medical indications, there are advantages in developing plasmids that have lower CpG content to reduce methylation and inactivation of expression and to reduce inappropriate inflammation through stimulation of the innate immune system in gene therapy. This application demonstrates that systemic delivery of plasmid nanoparticles, which have been precisely formulated to reduce free polymer and designed to reduce CpG content in the plasmid backbone and the gene of interest, leads to a therapeutic effect in treating cancer. The response is not compromised by a reduction in CpG content. On the contrary, the response is more selective for the expressed gene of interest.

### SUMMARY

In one aspect, the present disclosure provides methods for treating cancer in a subject in need thereof, comprising administering to the subject a nucleic acid construct comprising an expression cassette, wherein the expression cassette comprises a cancer-specific promoter and one or more therapeutic genes.

In some embodiments, the cancer-specific promoter is the PEG-3 promoter. In some embodiments, the one or more therapeutic genes is a cytokine, a thymidine kinase, a toxin, a pathogen-associated molecular pattern (PAMP), a danger-associated molecular pattern (DAMP), an immune checkpoint inhibitor gene, or any combination thereof.

In some embodiments, the thymidine kinase is HSV1-TK. 5. In some embodiments, the PAMP is flagellin (FliC). In some embodiments, the cytokine is a single chain variant of IL-12 (scIL-12).

In some embodiments, if multiple therapeutic genes are present, the multiple therapeutic genes are separated by a picornavirus 2A ribosome skipping sequence. In some embodiments, the picornavirus ribosome skipping sequence is P2A or T2A.

In some embodiments, the therapeutic gene is engineered to have a reduced CpG content compared to its wild-type counterpart. In some embodiments, the nucleic acid construct comprises a CpG-free plasmid backbone.

In some embodiments, the nucleic acid construct is formulated into nanoparticles with a cationic polymer. In some embodiments, the cationic polymer is linear polyethylenimine. In some embodiments, the nanoparticles are prepared at a N/P ratio of 4 or 6. In some embodiments, the nanoparticles are lyophilized. In some embodiments, the nucleic acid construct is delivered systemically.

In some embodiments, the cancer is selected from the group consisting of breast cancer, melanoma, carcinoma of unknown primary (CUP), neuroblastoma, malignant glioma, cervical cancer, colon cancer, hepatocarcinoma, ovarian cancer, lung cancer, pancreatic cancer, and prostate cancer.

In some embodiments, the immune checkpoint inhibitor gene encodes a monoclonal antibody selected from the group consisting of an anti-PD-1 antibody, an anti-PD-L1 antibody, and an anti-CTLA-4 antibody. In some embodiments, the immune checkpoint inhibitor gene encodes an immune checkpoint inhibitor fusion protein comprising a PD-1 fusion protein. In some embodiments, the PD-1 fusion protein comprises a fusion of PD-1 and an immunoglobulin Fc region. In some embodiments, the cytokine is selected from the group consisting of IL-12, IL-24, IL-2, IL-15, and GM-CSF.

In one aspect, the present disclosure provides nucleic acid constructs for the treatment of cancer comprising an expression cassette, wherein the expression cassette comprises a cancer-specific promoter and one or more therapeutic genes.

In some embodiments, the cancer-specific promoter is the PEG-3 promoter. In some embodiments, the one or more therapeutic genes is a cytokine, a thymidine kinase, a toxin, a pathogen-associated molecular pattern (PAMP), a danger-associated molecular pattern (DAMP), an immune checkpoint inhibitor gene, or any combination thereof.

In some embodiments, the thymidine kinase is HSV1-TK. In some embodiments, the PAMP is flagellin (FliC).

In some embodiments, if multiple therapeutic genes are present, the multiple therapeutic genes are separated by a picornavirus 2A ribosome skipping sequence. In some embodiments, the picornavirus ribosome skipping sequence is P2A or T2A.

In some embodiments, the therapeutic gene is engineered to have a reduced CpG content compared to its wild-type counterpart. In some embodiments, the nucleic acid construct comprises a CpG-free plasmid backbone.

In some embodiments, the nucleic acid construct is formulated into nanoparticles with a cationic polymer. In some embodiments, the cationic polymer is linear polyethylenimine. In some embodiments, the nanoparticles are prepared at a N/P ratio of 4 or 6. In some embodiments, the nanoparticles are lyophilized. In some embodiments, the nucleic acid construct is delivered systemically.

In some embodiments, the cancer is selected from the group consisting of breast cancer, melanoma, carcinoma of unknown primary (CUP), neuroblastoma, malignant glioma, cervical cancer, colon cancer, hepatocarcinoma, ovarian cancer, lung cancer, pancreatic cancer, and prostate cancer.

In some embodiments, the immune checkpoint inhibitor gene encodes a monoclonal antibody selected from the group consisting of an anti-PD-1 antibody, an anti-PD-L1 antibody, and an anti-CTLA-4 antibody. In some embodiments, the immune checkpoint inhibitor gene encodes an immune checkpoint inhibitor fusion protein comprising a PD-1 fusion protein.

In some embodiments, the PD-1 fusion protein comprises a fusion of PD-1 and an immunoglobulin Fc region. In some embodiments, the cytokine is selected from the group consisting of IL-12, IL-24, IL-2, IL-15, and GM-CSF. In some embodiments, the cytokine is a single chain variant of IL-12 (scIL-12).

In one aspect, the present disclosure provides compositions for the treatment of cancer comprising an expression cassette, wherein the expression cassette comprises a cancer-specific promoter and one or more therapeutic genes.

In some embodiments, the cancer-specific promoter is the PEG-3 promoter. In some embodiments, the one or more therapeutic genes is a cytokine, a thymidine kinase, a toxin, a pathogen-associated molecular pattern (PAMP), a danger-associated molecular pattern (DAMP), an immune checkpoint inhibitor gene, or any combination thereof.

In some embodiments, the thymidine kinase is HSV1-TK. In some embodiments, the PAMP is flagellin (FliC).

In some embodiments, if multiple therapeutic genes are present, the multiple therapeutic genes are separated by a picornavirus 2A ribosome skipping sequence. In some embodiments, the picornavirus ribosome skipping sequence is P2A or T2A.

In some embodiments, the therapeutic gene is engineered to have a reduced CpG content compared to its wild-type counterpart. In some embodiments, the nucleic acid construct comprises a CpG-free plasmid backbone.

In some embodiments, the nucleic acid construct is formulated into nanoparticles with a cationic polymer. In some embodiments, the cationic polymer is linear polyethylenimine. In some embodiments, the nanoparticles are prepared at a N/P ratio of 4 or 6. In some embodiments, the nanoparticles are lyophilized. In some embodiments, the nucleic acid construct is delivered systemically.

In some embodiments, the cancer is selected from the group consisting of breast cancer, melanoma, carcinoma of unknown primary (CUP), neuroblastoma, malignant glioma, cervical cancer, colon cancer, hepatocarcinoma, ovarian cancer, lung cancer, pancreatic cancer, and prostate cancer.

In some embodiments, the immune checkpoint inhibitor gene encodes a monoclonal antibody selected from the group consisting of an anti-PD-1 antibody, an anti-PD-L1 antibody, and an anti-CTLA-4 antibody. In some embodiments, the immune checkpoint inhibitor gene encodes an immune checkpoint inhibitor fusion protein comprising a PD-1 fusion protein. In some embodiments, the PD-1 fusion protein comprises a fusion of PD-1 and an immunoglobulin Fc region. In some embodiments, the cytokine is selected from the group consisting of IL-12, IL-24, IL-2, IL-15, and GM-CSF. In some embodiments, the cytokine is a single chain variant of IL-12 (scIL-12).

The present technology generally relates to genetic constructs and methods for their use in cancer treatment. The gene constructs used in these methods comprise a promoter that is specifically or selectively active in cancer cells. These promoters may be referred to herein as “cancer promoters” or “cancer specific/selective promoters” or simply as “specific/selective promoters”. Due to the specificity afforded by these promoters, compositions, which include the constructs of the invention, can be advantageously administered systemically to a subject that is in need of cancer treatment. As used herein, the terms “cancer-specific promoter” and “cancer-selective promoter” are used interchangeably.

The present technology provides methods and compositions for precise delivery of anti-tumor agents to cancer cells and the tumor microenvironment, even when delivery is made systemically, since the anti-tumor agents associated with the methods are only expressed within cancer cells. This advantageously results in few or no side effects for patients being treated by the method, as opposed to the severe toxicity that has been observed in systemic treatment with anti-cancer agents such as IL-12 (Car et al., 1999, Tox. Pathology 27, 58-63). Systemic delivery enables the possibility to act on more than one tumor site in parallel and at an early stage, which is particularly relevant for metastatic disease.

In some embodiments, the present technology provides methods of treating tumors, cancerous cells, or cancerous tissues in a subject in need thereof. The method comprises administering to the subject a nucleic acid construct comprising a therapeutic gene operably linked to a cancer specific or cancer selective promoter. In another embodiment an additional step includes administering a prodrug which is activated by a therapeutic gene. In some embodiments, at least one, and possibly both, of the steps of administering may be carried out systemically. In some embodiments, the nucleic acid construct is present in a polyplex with a cationic polymer, such as polyethylenimine. In some embodiments, the tumors, cancerous tissues or cells include cancer cells of a type selected from groups consisting of breast cancer, melanoma, carcinoma of unknown primary (CUP), neuroblastoma, malignant glioma, cervical, colon, hepatocarcinoma, ovarian, lung, pancreatic, and prostate cancer. In some embodiments, the nucleic acid construct is present in a plasmid. In other embodiments, the nucleic acid construct is present in a viral vector such as a conditionally replication-competent adenovirus. In some embodiments, the cancer specific or cancer selective promoter is progression elevated gene-3 (PEG-3) promoter. In some embodiments, the gene encoding an anti-tumor agent is operably linked to a tandem gene expression element, for example, a ‘ribosome skipping’ 2A peptide sequence or an internal ribosomal entry site (IRES) that allows expression of multiple therapeutic genes. In other embodiments, the gene encoding an anti-tumor agent is operably linked to a cancer specific or cancer selective promoter. The anti-tumor agent may be HSV1-TK, mda-7/IL-24, IL-2, IL-12, GM-CSF, IL-15 or another cytokine or combinations of cytokines, for example.

In one aspect, the present disclosure provides a nucleic acid construct for the treatment of cancer, comprising an expression cassette comprising a cancer-specific promoter and a nucleic acid sequence encoding an immune checkpoint inhibitor fusion protein.

In some embodiments, the cancer-specific promoter is the PEG-3 promoter. In some embodiments, the therapeutic gene is a nucleic acid construct comprising a sequence encoding HSV1-TK. In some embodiments, the therapeutic gene is a nucleic acid construct comprising a sequence encoding HSV1-TK variant SR39. In some embodiments, the therapeutic gene is a nucleic acid construct comprising a sequence encoding the sodium iodide symporter (NIS). In some embodiments, the therapeutic gene is a nucleic acid construct comprising a sequence encoding a cytokine. In some embodiments, the cytokine is selected from the group consisting of IL-12, IL-24, IL-2, IL-15, and GM-CSF. In some embodiments, the therapeutic gene is a nucleic acid construct comprising a checkpoint inhibitor comprised of a fusion of an antibody heavy chain and light chain against PD-1 or CTLA-4 or PD-L1. In some embodiments, the therapeutic gene is an immune checkpoint inhibitor fusion protein comprising a PD-1 fusion protein. In some embodiments, the PD-1 fusion protein comprises a fusion of PD-1 and an immunoglobulin Fc region. In some embodiments, there are multiple therapeutic genes expressed from a single PEG-3 promoter and linked through picornaviral 2A ribosome skipping sequences.

In some embodiments, the construct comprises a plasmid that has been modified to have reduced CpG content. In some embodiments, the construct comprises a CpG-free plasmid backbone. In some embodiments, the construct comprises a nanoplasmid. In some embodiments, the construct comprises a minicircle. In some embodiments, the nucleic acid construct further comprises a picornavirus 2A ribosome skipping sequence. In some embodiments, the nucleic acid construct further comprises an IRES tricistronic cassette.

In some embodiments, the cytokine is expressed as a single-chain construct. In some embodiments, the construct is formulated into a nanoparticle. In some embodiments, the nucleic acid construct is present in a polyplex with a cationic polymer. In some embodiments, the cationic polymer is polyethylenimine.

In some embodiments, the cancer is selected from a group consisting of breast cancer, melanoma, carcinoma of unknown primary (CUP), neuroblastoma, malignant glioma, cervical cancer, colon cancer, hepatocarcinoma, ovarian cancer, lung cancer, pancreatic cancer, and prostate cancer.

### BRIEF DESCRIPTION OF THE DRAWINGS

FIGS. 1A-1C are diagrams of exemplary expression cassettes of the constructs of the present technology, disclosed herein, for use in therapeutic applications. Each CpG-free expression cassette is driven by the cancer specific activity of PEG-3. Cassettes are shown including a therapeutic gene, such as a cytokine or a gene such as thymidine kinase (HSV1-TK) or a checkpoint inhibitor and a toxin or a pathogen associated molecular pattern (PAMP), such as flagellin (FliC). X, Y, and Z, and can be any combination of the above. Each is separated by a picornavirus ribosome skipping sequence, such as P2A or T2A and a Furin-GSG site where removal of the 2A sequence is required. Cloning sites useful in the construction of the expression cassettes are shown.

FIGS. 2A-2B show expression of cytokine gene constructs in CpG-free plasmid backbone, formulated into nanoparticles with linear polyethylenimine and expressed in human lung cancer cell line, NCI-H460. The expression cassettes are shown for each PEG-3-TKISR39-cytokine plasmid construct used in transfections (FIG. 2A). FIG. 2B shows cytokine expression, as determined by ELISA, in the cell culture supernatant from transfected H460 cells.

FIGS. 3A-3B show human IL-2 and murine IL-12 expression levels from a cassette containing three payload genes expressed from a single PEG-3 promoter (PEG-TK-hIL2-mIL12) in H460 cells, as determined by ELISA. PEG-TK control (HSV1-TK; no IL2 or IL12) or cassettes containing either PEG-TK-hIL2 or PEG-TK-mIL12 are also shown as controls for the specificity of the antibodies used in the ELISA. Results from the anti-human IL-2 ELISA are shown in the left-hand panel and anti-murine IL-12 ELISA are shown in the panel on the right.

FIGS. 4A-4B show expression levels of murine IL-12 and human IL-15 from a three-gene cassette (PEG-TK-mIL12-hIL15 or PEG-SR39-mIL12-hIL15) cloned into a CpG-free plasmid transfected in H460 cells, as determined by ELISA, FIG. 4B. PEG-TK (PEG-3 HSV1-TK) or PEG-SR39 plasmids are provided as negative controls for each antibody. Cassettes were constructed with two P2A sites (TK-mIL12-hIL15; SR39-mIL12-hIL15) or one P2A and one T2A site (TK-hIL15-mIL12; SR39-hIL15-mIL12). The left-hand panel shows the ELISA data from the anti-murine IL-12 assay, the right-hand panel for the anti-human IL-15 assay.

FIG. 5 shows the expression of FliC domains from a cassette containing murine IL-12 and flagellin domains, as determined by Western blot using anti-FliC antibody. The expression of FliC can be seen as an obvious band in lanes PEG-TK-mIL12-flag and PEG-SR39-mIL12-flag. The predicted unglycosylated molecular weight is 39.5 kDa. Non-expressing empty plasmid pGL3 or constructs containing PEG-3 HSV1-TK (PEG-TK or PEG-TK with a 3′ BamHI cloning site) or PEG-SR39 are shown as negative controls.

FIGS. 6A-6D. Plasmid expression cassettes that were ligated into a CpG-free plasmid backbone and formulated into nanoparticles are shown in FIG. 6A. The biological activity of the formulated nanoparticles was tested in in vitro assays. FIG. 6B shows a cytotoxicity assay for the effect of ganciclovir, which is phosphorylated by HSV1-TK and causes cell death, resulting in an increase in fluorescence (in RFU) in this assay. The curves show that active HSV1-TK was expressed by plasmids PEG-TK-hIL2-mIL12, PEG-TK-mIL12, PEG-TK-mIL2-mIL12, but not PEG-lucia, in LL/2 cells as determined by an increase in fluorescence, correlating with cell death. FIG. 6C shows a cell proliferation assay demonstrating the proliferation of murine CTLL2 T cells following stimulation with cytokines. The x-axis shows a dilution series of cell culture supernatant and the luminescence reading (in RLU) on the y-axis reflects the relative number of CTLL2 cells 48 h after transfection with the listed nanoparticle formulations. Proliferation was observed with all cytokine containing plasmids (PEG-TK-hIL2-mIL12, PEG-mIL2-mIL12, PEG-TK-mIL2-mIL12) but not with PEG-lucia (negative control). FIG. 6D: PBMC proliferation to test functional activity of mIL-12 captured from supernatants of LL/2 cells transfected with the listed nanoparticle formulations. Proliferation of human PBMCs from two human donors (301 and 303) occurred in all formulations expressing murine IL-12 but not in the control (PEG-lucia), which expresses an irrelevant payload. The x-axis shows a dilution series of the culture supernatants used as a source of captured IL-12 and the luminescence reading (in RLU) on the y-axis reflects the relative number of cells.

FIG. 7 shows a Kaplan Meier survival plot of anti-tumor activity of the nanoparticles containing the indicated PEG-3 plasmids in C57BL/6 mice inoculated with an orthotopic LL/2 Red-FLuc model of lung cancer. Mice were dosed at 4-day intervals, beginning at day 5 (post tumor cell inoculation), as indicated by the arrows above the chart. The study was terminated on Day 23. Both PEG-TK-hIL2-mIL12 and PEG-mIL12 significantly (Log rank test, p≤0.001) extended survival in this model compared to the vehicle control (Trehalose) and PEG-lucia.

FIG. 8. Anti-tumor activity of PEG-3 nanoparticles used in the study shown in FIG. 7 was assessed through comparison of the mean in vivo luminescence signal±SEM (Total Flux (p/s)) in the lungs of mice at day 13 after implantation of LL/2 Red-FLuc cells orthotopically into the lungs of C57BL/6 mice. The luminescence signal is indicative of tumor cell growth. There was a significant reduction in signal (Dunnett's multiple comparisons test, p≤0.05) in the PEG-mIL12 group compared to the Trehalose vehicle control group and the PEG-Lucia nanoparticle control, indicating significant inhibition of tumor growth.

FIG. 9 shows a Kaplan Meier survival plot of anti-tumor activity in vivo of the nanoparticles containing the indicated PEG-3 plasmids in an orthotopic LL/2 Red-Fluc model of lung cancer in mice. Days at which nanoparticles were dosed post tumor cell inoculation are indicated by the arrows above the plot. Nanoparticles PEG-mIL12, PEG-TK-mGMCSF, PEG-TK-hIL15-mIL12 and PEG-TK-IL12-flag, but not PEG-lucia, significantly (p≤0.05, Log rank test) improved survival of the mice longer compared to the 9.5% Trehalose vehicle control.

FIG. 10 shows a Kaplan Meier survival plot of anti-tumor activity in vivo of nanoparticles containing the indicated PEG-3 plasmids in a B16F10-Red-FLuc experimental model of metastatic lung cancer. Nanoparticles were dosed at 3-day intervals, beginning at day 5 (post tumor cell inoculation) as indicated by the arrows above the plot. Nanoparticle formulations PEG-mIL12, PEG-TK-mIL12, PEG-mIL2-mIL12, PEG-TK-mIL2-mIL12 significantly extended survival (p≤0.05, Log rank test) of the mice compared to vehicle control and PEG-lucia. PEG-lucia also significantly extended survival in this study compared to the vehicle control.

FIG. 11. Anti-tumor activity of PEG-3 nanoparticles used in the experimental metastasis study (shown in FIG. 10) were assessed through comparison of the mean in vivo luminescence signal±SEM (Total Flux (p/s)) in the lungs of C57BL/6 mice 12 days after inoculation of B16F10-Red-FLuc cells. The luminescent signal is indicative of the growth of tumor cells expressing firefly luciferase. There was a significant reduction in signal between PEG-mIL2-mIL12 and the vehicle control group, and PEG-TK-mIL2-mIL12 and the vehicle control group (Dunnett's multiple comparisons test, p≤0.05).

FIG. 12 shows a Kaplan Meier survival plot of anti-tumor activity in vivo of two preparations (N/P=4 and N/P=6) of PEG3-mIL-12 nanoparticles in a B16F10-Red-Fluc experimental model of metastatic lung cancer. Nanoparticles were dosed at 3 day intervals, beginning at day 5 (post tumor cell inoculation) as indicated by the arrows above the plot. The nanoparticle formulations produced a significant survival benefit over vehicle control (Trehalose) (p≤0.01, Log rank test) and over the recombinant murine IL-12 (p≤0.05, Log rank test) at the dose tested.

FIG. 13. Anti-tumor activity of PEG-3 nanoparticles used in the experimental metastasis study (shown in FIG. 12) were assessed through comparison of the mean in vivo luminescence signal±SEM (Total Flux (p/s)) in the lungs of C57BL/6 mice 19 days after inoculation of B16F10-Red-FLuc cells. The luminescent signal is indicative of the growth of tumor cells expressing firefly luciferase. There was a significant reduction in signal between PEG-mIL12 (N/P=6) and the vehicle control groups, and recombinant mIL-12 and the vehicle control groups (Dunnett's multiple comparisons test, p≤0.05) and a trend towards significance (p=0.0538) for PEG-mIL12 (N/P=4).

FIG. 14. Two plasmids were used to determine tumor specific expression in the context of CpG burden of the PEG-3 containing plasmids: one plasmid, pGL3-PEG3-fluc, a firefly luciferase gene whose expression is driven by the PEG-3 promoter, contains 357 CpG sites and pCpGfree-PEG-fluc, which is free of CpG sites (including the luciferase gene) with the exception of 43 CpG-sequences within the PEG3 promoter. Formulated nanoparticles were injected into NSG mice, non-tumor bearing or containing LL/2 or B16F10 tumors. BLI imaging was performed 48 h post-injection of the nanoparticles. The region of interest was drawn to cover the entire lung region of each mouse and total flux (photon counts/sec) was calculated to determine the expression of the fLuc. The counts for individual mice treated with either plasmid were grouped. In both the LL/2 and B16F10 models, the pCpGfree-PEG-fluc groups have significantly more counts, corresponding to greater expression of firefly luciferase than in animals treated with the pGL3-PEG3-fluc plasmid. There was no significant difference between the two plasmids in terms of luciferase expression in healthy animals.

FIG. 15. Twelve animals (CD34⁺HU-NSG™ mice from a single human umbilical cord donor) were inoculated with of 10⁶ MDA-MB-231-luc2 cells and tumor growth was confirmed on Day 8 using BLI imaging of the lungs. PEG-lucia, PEG-hIL12 and PEG-IL24 nanoparticles were dosed on study days 4, 7, 10, 13, 16, and 19 and the animals were monitored over a period of 32 days and survival was noted. As observed from the survival data, individual animals treated with nanoparticles containing the human IL-12 and human IL-24 plasmids (PEG-hIL12 and PEG-hIL24, respectively) survived longer compared to animals in the control groups.

### DETAILED DESCRIPTION

It is to be appreciated that certain aspects, modes, embodiments, variations and features of the technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this technology belongs.

As used in this specification and the appended claims, the singular forms “a”, “an” and “the” include plural referents unless the content clearly dictates otherwise. For example, reference to “a cell” includes a combination of two or more cells, and the like.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, a nucleic acids having a “reduced” CpG content refers to a nucleic acid engineered to have a reduced number of CpG motifs compared to its wildtype counterpart. In some embodiments, the reduced CpG nucleic acid is a vector. In some embodiments the vector is used for the delivery of therapeutic genes to a subject. In some embodiments, the vector is a viral vector. In some embodiments, the vector is a plasmid. In some embodiments, the reduced CpG nucleic acid is a therapeutic gene or a reporter gene. In some embodiments, the reduced CpG therapeutic gene is a cytokine. In some embodiments, the reduced CpG cytokine is IL-12.

As used herein, “CpG-free” refers to a nucleic acid construct having no CpG motifs. In some embodiments, the CpG-free nucleic acid is a vector. In some embodiments the vector is used for the delivery of therapeutic genes to a subject. In some embodiments, the vector is a viral vector. In some embodiments, the vector is a plasmid. In some embodiments, a CpG-free plasmid vector is referred to as a “CpG-free plasmid backbone.” In some embodiments, the CpG-free nucleic acid is a therapeutic gene or a reporter gene. In some embodiments, the CpG-free therapeutic gene is a cytokine. In some embodiments, the CpG-free cytokine is IL-12.

### Compositions and Methods

As discussed herein, cancer-specific promoters can be used for targeted expression of reporter and therapeutic genes in a subject having cancer. For example, U.S. patent application Ser. No. 13/881,777 (U.S. Patent Pub. 20130263296), the contents of which are hereby incorporated by reference, shows that the expression of reporter genes driven by the PEG-3 promoter allows for exceptionally sensitive cancer imaging. The PEG-3 promoter is widely accepted in the field to be a universal cancer-specific promoter and is highly effective for cancer therapeutic applications.

The present disclosure relates to improved therapeutic constructs for the treatment of cancer. In some embodiments, the constructs comprise a PEG-3 promoter and a first gene. In some embodiments, the constructs further comprise a second gene. In some embodiments, the constructs further comprise a third gene.

In some embodiments, the first gene comprises a cytokine. Illustrative cytokines include interferons and interleukins such as interleukin 1 (IL-1), IL-2, IL-3, IL-4, IL-5, IL-6, IL-7, IL-8, IL-9, IL-10, IL-11, IL-12, IL-13, IL-14, IL-15, IL-18, β-interferon, α-interferon, γ-interferon, angiostatin, thrombospondin, endostatin, GM-CSF, G-CSF, M-CSF, METH 1, METH 2, tumor necrosis factor, TGFβ, LT and combinations or fusions thereof, for example IL-2 and IL-12 both fused to the same Fc domain (see e.g., Hombach &, Abken Oncoimmunology 2; e23205, 2013).

In some embodiments, therapeutic constructs of the present technology comprise other anti-tumor agents, including, for example, but not limited to, interleukins, chemokines, tumor necrosis factor (TNF); interferon-beta and virus-induced human Mx proteins; TNF alpha and TNF beta; human melanoma differentiation-associated gene-7 (mda-7), also known as interleukin-24 (IL-24), various truncated versions of mda-7/IL-24 such as M4; siRNAs and shRNAs targeting important growth regulating or oncogenes which are required by or overexpressed in cancer cells; antibodies such as antibodies that are specific or selective for attacking cancer cells, chemokines important for the recruitment of leukocytes such as CXCL9, CXCL10, or CXCL11, etc.

In some embodiments, the second and/or third gene encodes another cytokine. Illustrative cytokines include interferons and interleukins such as interleukin 1 (IL-1), IL-2, IL-3, IL-4, IL-5, IL-6, IL-7, IL-8, IL-9, IL-10, IL-11, IL-12, IL-13, IL-14, IL-15, IL-18, β-interferon, α-interferon, γ-interferon, angiostatin, thrombospondin, endostatin, GM-CSF, G-CSF, M-CSF, METH 1, METH 2, tumor necrosis factor, TGFβ, LT and combinations or fusions thereof, for example IL-2 and IL-12 both fused to the same Fc domain (see e.g., Hombach &, Abken Oncoimmunology 2; e23205, 2013). Other anti-tumor agents include: interleukins, chemokines, tumor necrosis factor (TNF); interferon-beta and virus-induced human Mx proteins; TNF alpha and TNF beta; human melanoma differentiation-associated gene-7 (mda-7), also known as interleukin-24 (IL-24), various truncated versions of mda-7/IL-24 such as M4; siRNAs and shRNAs targeting important growth regulating or oncogenes which are required by or overexpressed in cancer cells; antibodies such as antibodies that are specific or selective for attacking cancer cells, etc.

In some embodiments, the second or third gene comprises a nucleic acid sequence encoding a therapeutic molecule. In some embodiments, the therapeutic molecule comprises a cytokine. In some embodiments, the second gene comprises a nucleic acid sequence encoding a fragment of PD-1 or a PD-1 fusion protein. In some embodiments, the fusion includes the extracellular region of PD-1. In some embodiments, the fusion protein comprises a PD-1-immunoglobulin Fc fusion protein. Additionally or alternatively, in some embodiments, the fusion includes one or more of the following molecules: proteins, polypeptides, antibodies or nucleic acid aptamers that bind to and either antagonize or agonise LAG-3, CTLA-4, CD80, CD86, PD-L1, PD-L2, CD48, CD244, TIM-3, Siglecs, HVEM, BTLA, CD160, CD40, CD40L, CD27, 4-1BB, OX40, GITR, VISTA B7-H3, B7-H4, KIRs, NKG2D, NKG2A, MICA, MICB, etc. as described by Mahoney, et al. (Nature Reviews Drug Discovery, 14, 561-565, 2015). In some embodiments, the selection of molecule will depend on whether immune cell activation or repression is required, as is well-known in the art. Additionally or alternatively, in some embodiments, Fc fusions may trap cytokines (see e.g., Huang Current Opinion in Biotechnology, 20:692-699, 2009). Additionally or alternatively, in some embodiments, the fusion protein does not include an Fc sequence. By way of example, but not by way of limitation, in some embodiments, fusion proteins includes PD-1, or the extracellular region of PD-1, and one or more of the NC2 domain of Fibril Associated Collagens with Interrupted Triple helices (FACIT) collagen trimerization domain, non-collagenous domain (NCI) of human collagen XVIII or its trimerization domain (TD) (Boudko and Bachinger J Biol Chem. 287:44536-45, 2012), a C4 bp oligomerization domain (Spencer, et al., PLoS One 7:e33555, 2012) or other coiled-coil domains (Apostolovic, et al., Chem Soc Rev. 39:3541-75, 2010).

Illustrative genes and nucleic acid sequences for use in therapeutic constructs provided herein are described in, for example, U.S. Pat. Nos. 8,163,528, 7,507,792, 5,994,104, 5,846,767, 5,698,520, and 5,629,204.

The present technology provides nucleic acid constructs and methods for their use in cancer treatment. Constructs designed for therapy generally comprise a cancer-specific promoter and a recombinant gene that encodes a therapeutic agent (e.g. a protein or polypeptide whose expression is detrimental to cancer cells) operably linked to the cancer-specific promoter. Thus, targeted killing of cancer cells occurs even when the constructs are administered systemically. These constructs and methods, and various combinations and permutations thereof, are discussed in detail below.

The constructs of the present technology include at least one transcribable element (e.g. a gene composed of sequences of nucleic acids) that is operably connected or linked to a promoter that specifically or selectively drives transcription within cancer cells. Expression of the transcribable element may be inducible or constitutive. Illustrative cancer selective/specific promoters (and or promoter/enhancer sequences) that may be used include but are not limited to: PEG-3, astrocyte elevated gene 1 (AEG-1) promoter, surviving promoter, human telomerase reverse transcriptase (hTERT) promoter, hypoxia-inducible promoter (HIP-1-alpha), DNA damage inducible promoters (e.g. GADD promoters), metastasis-associated promoters (metalloproteinase, collagenase, etc.), ceruloplasmin promoter (Lee, et al., Cancer Res. 64; 1788, 2004), mucin-1 promoters such as DF3/MUC1 (see U.S. Pat. No. 7,247,297), HexII promoter as described in US patent application 2001/00111128; prostate-specific antigen enhancer/promoter (Rodriguez, et al. Cancer Res., 57: 2559-2563, 1997); α-fetoprotein gene promoter (Hallenbeck, et al. Hum. Gene Ther., 10: 1721-1733, 1999); the surfactant protein B gene promoter (Doronin, et al. J. Virol., 75: 3314-3324, 2001); MUC1 promoter (Kurihara, et al. J. Clin. Investig., 106: 763-771, 2000); H19 promoter as per U.S. Pat. No. 8,034,914; those described in issued U.S. Pat. Nos. 7,816,131, 6,897,024, 7,321,030, 7,364,727, and others, etc., as well as derivative forms thereof.

Any promoter that is specific for driving gene expression in cancer cells only, or that is selective for driving gene expression in cancer cells, or at least in cells of a particular type of cancer (so as to treat primary and metastatic cancer in prostate, colon, breast, etc.) may be used in the practice of the present technology. As will be understood by one of skill in the art, promoters that drive gene expression specifically in cancer cells are those that, when operably linked to a gene, function to promote transcription of the gene only in a cancerous cell, and not in non-cancerous cells. As will further be understood by one of skill in the art, promoters that are selective for driving gene expression in cancer cells are those that, when operably linked to a gene, function to promote transcription of the gene to a greater degree in a cancer cell than in a non-cancerous cell. For example, the promoter drives gene expression of the gene at least about 2-fold, or about 3-, 4-, 5-, 6-, 7-, 8-, 9-, or 10-fold, or even about 20-, 30-, 40-, 50-, 60-, 70-, 80-, 90- or 100-fold or more (e.g. 500- or 1000-fold) when located within a cancerous cell than when located within a non-cancerous cell, when measured using standard gene expression measuring techniques that are known to those of skill in the art.

In one embodiment, the promoter is the PEG-3 promoter or a functional derivative thereof. This promoter is described in detail, for example, in issued U.S. Pat. No. 6,737,523, the complete contents of which are herein incorporated by reference. In some embodiments, a “minimal” PEG-3 promoter is utilized, i.e. a minimal promoter that includes a PEA3 protein binding nucleotide sequence, a TATA sequence, and an AP1 protein binding nucleotide sequence, for example, the sequence depicted in, as described in U.S. Pat. No. 6,737,523, Nucleotide sequences which display homology to the PEG-3 promoter and the minimal PEG-3 promoter sequences are also encompassed for use, e.g. those which are at least about 50, 60, 70, 75, 80, 85, 90, 95, 96, 97, 98, or 99% homologous, as determined by standard nucleotide sequence comparison programs which are known in the art.

In some embodiments, the present technology provides vectors for delivery of therapeutic genes. In some embodiments, the vector is a viral vector. In some embodiments, the vector is a non-viral vector.

Illustrative non-viral vectors include but are not limited to, for example, cosmids or plasmids; and, particularly for cloning large nucleic acid molecules, bacterial artificial chromosome vectors (BACs) and yeast artificial chromosome vectors (YACs); as well as liposomes (including targeted liposomes); cationic polymers; ligand-conjugated lipoplexes; polymer-DNA complexes; poly-L-lysine-molossin-DNA complexes; chitosan-DNA nanoparticles; polyethylenimine (PEI, e.g. linear, branched or functionalized PEI)-DNA complexes; PLGA (poly(lactic-co-glycolic acid)); PBAEs (poly β-amino esters); various nanoparticles and/or nanoshells such as multifunctional nanoparticles, metallic nanoparticles or shells (e.g. positively, negatively or neutral charged gold particles, cadmium selenide, etc.); ultrasound-mediated microbubble delivery systems; various dendrimers (e.g. polyphenylene and poly(amidoamine)-based dendrimers; etc (Rodriguez Gascon, et al., 2013, Non-Viral Delivery Systems in Gene Therapy, Gene Therapy—Tools and Potential Applications, Dr. Francisco Martin (Ed.), InTech; Green et al., 2007, Adv. Mater. 19, 2836-2842).

Illustrative viral vectors include but are not limited to: bacteriophages, various baculoviruses, retroviruses, and the like. Those of skill in the art are familiar with viral vectors that are used in “gene therapy” applications, which include but are not limited to: Herpes simplex virus vectors (Geller, et al., Science, 241:1667-1669, 1988); vaccinia virus vectors (Piccini, et al., Meth. Enzymology, 153:545-563, 1987); cytomegalovirus vectors (Mocarski, et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos, et al., Proc. Natl. Acad. Sci. USA, 85:6460-6464, 1988); Blaese, et al., Science, 270:475-479, 1995; Onodera, et al., J. Virol., 72:1769-1774, 1998); adenovirus vectors (Berkner, Biotechniques, 6:616-626, 1988; Cotten, et al., Proc. Natl. Acad. Sci. USA, 89:6094-6098, 1992; Graham, et al., Meth. Mol. Biol., 7:109-127, 1991; Li, et al., Human Gene Therapy, 4:403-409, 1993; Zabner, et al., Nature Genetics, 6:75-83, 1994); adeno-associated and hybrid adeno-associated virus vectors (Goldman, et al., Human Gene Therapy, 10:2261-2268, 1997; Greelish, et al., Nature Med., 5:439-443, 1999; Wang, et al., Proc. Nati. Acad. Sci. USA, 96:3906-3910, 1999; Snyder, et al., Nature Med., 5:64-70, 1999; Herzog, et al., Nature Med., 5:56-63, 1999; Choi, et al., Curr Gene Ther. 5: 299-310, 2005); retrovirus vectors (Donahue, et al., Nature Med., 4:181-186, 1998; Shackleford, et al., Proc. Natl. Acad. Sci. USA, 85:9655-9659, 1988; U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri, et al., Nature Genetics, 17:314-317, 1997), as well as viruses that are replication-competent conditional to a cancer cell such as oncolytic herpes virus NV 1066 and vaccinia virus GLV-1h68, as described in United States patent application 2009/0311664. In particular, adenoviral vectors may be used, e.g. targeted viral vectors such as those described in published United States patent application 2008/0213220.

Those of skill in the art will recognize that the choice of a particular vector will depend on the intended use, and will be selected according to vector properties known in the art.

Host cells which contain the constructs and vectors of the present technology are also encompassed, e.g. in vitro cells such as cultured cells, or bacterial or insect cells which are used to store, generate or manipulate the vectors, and the like. The constructs and vectors may be produced using recombinant technology or by synthetic means.

In some embodiments nucleic acid constructs described herein comprise a CpG-free plasmid, such as, for example, the Invivogen (San Diego, Calif., USA) pCpGfree vector. In some embodiments, constructs comprise a nanoplasmid, such as, for example, the Nature Technology Corporation (Lincoln, Nebr., USA) NTC9385R plasmid. In some embodiments, the nucleic acid construct comprises a minicircle (Chen, et al., Molecular Therapy 8: 495-500, 2003). Any suitable CpG-free plasmid, nanoplasmid, minicircle, or other expression vector may be used as components of the nucleic acid construct. In some embodiments, the nucleic acid construct is formulated into a nanoparticle.

The present technology provides compositions, which comprise one or more vectors or constructs as described herein and a pharmacologically acceptable carrier. The compositions are usually for systemic administration. The preparation of such compositions is known to those of skill in the art. Typically, they are prepared either as liquid solutions or suspensions, or as solid forms suitable for solution in, or suspension in, liquids prior to administration. The preparation may also be emulsified. The active ingredients may be mixed with excipients that are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present technology may contain any of one or more ingredients known in the art to provide the composition in a form suitable for administration. The final amount of vector in the formulations may vary. However, in general, the amount in the formulations will be from about 1-99%.

Targeted cancer therapy is carried out by administering the constructs, vectors, etc. as described herein to a patient in need thereof. In some embodiments, a gene encoding a therapeutic molecule, e.g. a protein or polypeptide, which is deleterious to cancer cells is operably linked to a cancer-specific promoter as described herein in a “therapeutic construct” or “therapeutic vector.” The therapeutic protein may kill cancer cells (e.g. by initiating or causing apoptosis), or may slow their rate of growth (e.g. may slow their rate of proliferation), or may arrest their growth and development or otherwise damage the cancer cells in some manner, or may even render the cancer cells more sensitive to other anti-cancer agents, etc. By way of example only and not by way of limitation, in some embodiments, one or more therapeutic genes (genes encoding therapeutic molecules) are provided in a nucleic acid expression construct, operably linked to a cancer-specific promoter. In some embodiments, the cancer specific promoter is PEG-3. Additionally or alternatively, in some embodiments, the expression construct includes one or more of a nucleic acid sequence encoding an immune checkpoint inhibitor fusion protein.

Genes encoding therapeutic molecules that may be employed in the present technology include but are not limited to, suicide genes, including genes encoding various enzymes; oncogenes; tumor suppressor genes; toxins; cytokines; oncostatins; TRAIL, etc. Illustrative enzymes include, for example, thymidine kinase (TK) and various derivatives thereof; TNF-related apoptosis-inducing ligand (TRAIL), xanthine-guanine phosphoribosyltransferase (GPT); cytosine deaminase (CD); hypoxanthine phosphoribosyl transferase (HPRT); etc. Illustrative tumor suppressor genes include neu, EGF, ras (including H, K, and N ras), p53, Retinoblastoma tumor suppressor gene (Rb), Wilm's Tumor Gene Product, Phosphotyrosine Phosphatase (PTPase), AdE1A and nm23. Suitable toxins include Pseudomonas exotoxin A and S; diphtheria toxin (DT); E. coli LT toxins, Shiga toxin, Shiga-like toxins (SLT-1, -2), ricin, abrin, supporin, gelonin, etc. Suitable cytokines include interferons and interleukins such as interleukin 1 (IL-1), IL-2, IL-3, IL-4, IL-5, IL-6, IL-7, IL-8, IL-9, IL-10, IL-11, IL-12, IL-13, IL-14, IL-15, IL-18, β-interferon, α-interferon, γ-interferon, angiostatin, thrombospondin, endostatin, GM-CSF, G-CSF, M-CSF, METH 1, METH 2, tumor necrosis factor, TGFβ, LT and combinations or fusions thereof, for example IL-2 and IL-12 both fused to the same Fc domain (see e.g., Hombach &, Abken Oncoimmunology 2; e23205 (2013)). Other anti-tumor agents include: interleukins, chemokines, tumor necrosis factor (TNF); interferon-beta and virus-induced human Mx proteins; TNF alpha and TNF beta; human melanoma differentiation-associated gene-7 (mda-7), also known as interleukin-24 (IL-24), various truncated versions of mda-7/IL-24 such as M4; siRNAs and shRNAs targeting important growth regulating or oncogenes which are required by or overexpressed in cancer cells; antibodies such as antibodies that are specific or selective for attacking cancer cells; etc.

When the therapeutic agent is TK (e.g. viral TK), a TK substrate such as acyclovir; ganciclovir; various thymidine analogs (e.g. those containing o-carboranylalkyl groups at the 3-position (Al-Madhoun, et al., Cancer Res. 64:6280-6, 2004) is administered to the subject. These drugs act as prodrugs, which in themselves are not toxic, but are converted to toxic drugs by phosphorylation by viral TK. Both the TK gene and substrate must be used concurrently to be toxic to the host cancer cell.

In some aspects, the present disclosure provides constructs for cancer therapy comprising a nucleic acid encoding an immune checkpoint inhibitor antibody or fusion protein that bind to any of the following molecules LAG-3, CTLA-4, CD80, CD86, PD-L1, PD-L2, CD48, CD244, TIM-3, Siglecs, HVEM, BTLA, CD160, CD40, CD40L, CD27, 4-1BB, OX40, GITR, VISTA B7-H3, B7-H4, KIRs, NKG2D, NKG2A, MICA, MICB, etc. as described by Mahoney, et al. (Nature Reviews Drug Discovery, 14, 561-565, 2015). In some embodiments, the DNA sequence encodes anti-CTLA-4 (Ipilimumab) or anti-PD-1 (Nivolumab or Pembrolizumab) or anti-PD-L1 (Durvalumab) immune checkpoint inhibitor antibody. In some embodiments, the fusion protein is a programmed cell death-1 (PD-1) fusion protein. In some embodiments, the fusion protein comprises PD-1 fused to an immunoglobulin Fc region.

As known in the art, PD-1 is an immunoglobulin superfamily cell surface receptor expressed on T cells and pro-B cells. Functioning as an immune checkpoint, PD-1 down regulates the activation of T-cells, reducing autoimmunity and promoting self-tolerance. The inhibitory effect of PD-1 is accomplished through a dual mechanism of promoting apoptosis in antigen specific T-cells and reducing apoptosis in regulatory (suppressor) T cells. Agents that inhibit PD-1 function activate the immune system and have been used to treat various types of cancer. Accordingly, it is advantageous to use a PD-1 fusion protein in conjunction with cytokines for the treatment of cancer.

Fusion proteins may be made and tested using techniques known in the art, including methodology outlined herein.

Extracellular regions of receptors have been fused and used as traps for cytokines and growth factors. The extracellular domain of PD-1 can likewise be used as a decoy for its interaction between membrane bound PD-1 and its membrane bound ligands PD-L1 and PD-L2 when expressed in a soluble form. The interaction between PD-1 and its ligands are known to be weak (low μM) (Cheng, et al. J. Biol. Chem. 288: 11771-11785, 2013), therefore fusion of the extracellular domain of PD-1 to the Fc portion of IgG provides additional benefit in that this increases the avidity of the molecule and its apparent affinity.

Additionally, fusion with IgG Fc will increase the molecular mass of the molecule and its hydrodynamic radius, thus increasing the circulating half-life of the PD-1 molecule. Half-life is also extended through binding the Brambell receptor (FcRn), which is involved in recycling antibodies back into circulation following internalization within cells. Fc regions from Igbo 1-4 or even other immunoglobulin classes such as IgA, IgE, IgM may be used. Exemplary, non-limiting Fc fusions are described by Huang, et al. (Current Opinion in Biotechnology 20:692-699, 2009).

The hinge region of the immunoglobulins positions the Fab regions to contact the antigen but also possesses the ability to interact with Fc receptors and proteins of the complement system. Fusion with the extracellular domain of PD-1 accommodates flexibility of the hinge region although this may be extended or shortened to provide optimal ligand binding. The sequence of the hinge region may be adapted to increase or decrease the affinity for Fcγ receptors as illustrated in WO2009/006520. Other effector properties of the Fc region may also be modified for example US2008/0227958A1, US2004/0132101A1, WO2007/041635A2, amongst others. In some embodiments, cytokines may additionally be fused to the Fc region, as illustrated in immunokine approaches (Pasche and Neri Drug Discovery Today 17, 583-590, 2012).

Simultaneous expression and secretion of the checkpoint inhibitor fusion molecule with HSV1-TK and/or a cytokine has the following benefits. First, the genes will be expressed locally at the tumor site as driven by the cancer specific promoter, therefore the effect will be localized to the tumor microenvironment. This will limit toxicity and immune-related adverse events. Second, irradiation and checkpoint inhibition has been shown to be synergistic (Deng, et al., J Clin Invest. 124:687-695, 2012), therefore conversion of a radiolabeled prodrug and expression of a checkpoint inhibitor within the tumor environment will also be synergistic and localized. Third, expression of a checkpoint inhibitor in isolation has improved CD4+ and CD8+ T cell responses but has limited clinical benefit (Amancha, et al., J Immunol. 191:6060-70, 2013). Engagement of the PD-1 molecule with its ligand on macrophages has been demonstrated to down regulate synthesis of IL-12 (Cho, et al., Immunology Letters 127:39-47, 2009), thus expression of cytokines will help to restore the immune response to abnormal cells. In particular, expression of PD-L1 has been correlated with poor prognosis in NSLC and poor survival of patients with solid tumors (Wang, et al., EJSO 41 450-456, 2015; Wu el al., PLoS ONE 10(6): e0131403, 2015) and blocking the binding of PD-L1 with membrane bound PD-1 or anti-PD-1 or anti-PD-L1 will interfere with the process on immune suppression.

Various TK enzymes or modified or mutant forms thereof may be used in the practice of the present technology, including but not limited to: HSV1-TK, HSV1-sr39TK, mutants with increased or decreased affinities for various substrates, temperature sensitive TK mutants, codon-optimized TK, the mutants described in U.S. Pat. No. 6,451,571 and US patent application 2011/0136221, both of which are herein incorporated by reference; various suitable human TKs and mutant human TKs, etc.

TK substrates that may be used include but are not limited to: analogues of guanosine, such as ganciclovir and valganciclovir; thymidine analogs, such as “fialuridine” i.e. [1-(2-deoxy-2-fluoro-1-D-arabinofuranosyl)-5-iodouracil], also known as “FIAU” and various forms thereof, e.g. 2′-fluoro-2′-deoxy-β-D-5-[¹²⁵I] iodouracil-arabinofuranoside ([¹²⁵I]FIAU), [¹²⁴I]FIAU; thymidine analogs containing o-carboranylalkyl groups at the 3-position, as described by Al Mahoud, et al., (Cancer Res, 64; 6280-6, 2004) and radiolabeled FXAU derivatives such as ¹³¹I-FIAU, ²¹¹At-FAAU.

Other proteins that may function as therapeutic molecules in the practice of the present technology are transporter molecules which are located on the cell surface or which are transmembrane proteins, e.g. ion pumps which transport various ions across cells membranes and into cells. An illustrative ion pump is the sodium-iodide symporter (NIS) also known as solute carrier family 5, member 5 (SLC5A5). In nature, this ion pump actively transports iodide (I) across e.g. the basolateral membrane into thyroid epithelial cells and can be used with radiolabeled iodide molecules, such as 1-131 Nat Recombinant forms of the transporter encoded by sequences of the constructs described herein may be selectively transcribed in cancer cells, and transport radiolabeled iodine into the cancer cells.

In some embodiments, the present technology provides methods for treating cancer. In some embodiments, the treatment involves administering to a cancer patient, or a subject having cancer, a gene construct (e.g. a plasmid). In this embodiment, expression of the therapeutic gene is mediated by a cancer cell specific or selective promoter as described herein. In some embodiments, the construct expresses at least two therapeutic genes and comprises two promoters in order to prevent or lessen the chance of crossover and recombination within the construct. In some embodiments, the construct comprises a single promoter. In some embodiments, the cancer-specific or cancer selective promoter is the PEG-3 promoter.

In some embodiments, tandem translation mechanisms may be employed, for example, the insertion of one or more internal ribosomal entry site (IRES) into the construct, which permits translation of multiple mRNA transcripts from a single mRNA. In this manner, more than one sequence encoding a therapeutic protein/polypeptide are selectively or specifically produced within the targeted cancer cells.

In some embodiments, the therapeutic gene comprises an IRES sequence. Natural IRES sequences may be used or synthetic or variant sequences that fit with an IRES containing a hairpin loop of a RNRA consensus are used (Robertson, et al., RNA 5:1167-1179, 1999). In some embodiments, therapeutic constructs comprise an IRES tricistronic cassette.

Alternatively, the polypeptides encoded by the constructs of the present technology (e.g. plasmids) may be genetically engineered to contain a contiguous sequence comprising two or more polypeptides of interest (e.g. a reporter and a toxic agent) with an intervening sequence that is cleavable within the cancer cell, e.g. a sequence that is enzymatically cleaved by intracellular proteases, or even that is susceptible to non-enzymatic hydrolytic cleavage mechanisms. In this case, cleavage of the intervening sequence results in production of functional polypeptides, i.e. polypeptides which are able to carry out their intended function, e.g. they are at least 50, 60, 70, 80, 90, or 100% (or possible more) as active as the protein sequences on which they are modeled or from which they are derived (e.g. a sequence that occurs in nature), when measured using standard techniques that are known to those of skill in the art.

In other embodiments of therapy, two different vectors may be administered in a single formulation.

In other embodiments of therapy, the genes of interest are encoded in the genome of a viral vector that is capable of transcription and/or translation of multiple rnRNAs and/or the polypeptides or proteins they encode, by virtue of the properties inherent in the virus. In this embodiment, such viral vectors are genetically engineered to contain and express genes of interest (e.g. therapeutic gene(s)) under the principle control of one or more cancer specific promoters.

In some aspects, the present disclosure provides a nucleic acid construct treatment of cancer. In some embodiments, the construct comprises a cancer-specific promoter, a first gene, a second gene, and a third gene. In some embodiments, the cancer-specific promoter is the PEG-3 promoter. In some embodiments, up to three therapeutic genes are expressed, any suitable cancer-specific promoter, reporter gene, immune checkpoint inhibitor fusion, and therapeutic gene may be used as components of the nucleic acid construct. In some embodiments, the reporter gene comprises a picornavirus 2A ribosome skipping sequence, which is typically characterized by a C-terminal D(V/I)ExNPGP motif (Sharma et al., Nucleic Acids Res., 40: 3143-3151, 2012).

In some embodiments, the therapeutic gene comprises HSV1-TK, an HSV1-TK splice variant, or an HSV1-TK mutant.

In some embodiments, the therapeutic gene comprises sequences encoding an immune checkpoint inhibitor protein that binds to any of the that bind to any of the following molecules LAG-3, CTLA-4, CD80, CD86, PD-L1, PD-L2, CD48, CD244, TIM-3, Siglecs, HVEM, BTLA, CD160, CD40, CD40L, CD27, 4-1BB, OX40, GITR, VISTA B7-H3, B7-H4, KIRs, NKG2D, NKG2A, MICA, MICB, etc. as described by Mahoney, et al. (Nature Reviews Drug Discovery, 14, 561-565). In some embodiments, the DNA sequence encodes anti-CTLA-4 (Ipilimumab) or anti-PD-1 (Nivolumab or Pembrolizumab) immune checkpoint inhibitor antibody. In some embodiments, the fusion protein is a programmed cell death-1 (PD-1) fusion protein. In some embodiments, the fusion protein comprises PD-1 fused to an immunoglobulin Fc region.

In some embodiments, the therapeutic gene comprises a cytokine. In some embodiments, the cytokine is selected from a group consisting of IL-12, IL-24, IL-2, IL-15, and GM-CSF. In some embodiments the cytokine is IL-12, formed as a single chain molecule so that the p35 and p40 proteins are expressed coordinately (Anderson, et al., Human Gene Therapy 8; 1125-1135, 1997).

In some embodiments, a second or third gene comprises of a pathogen associated molecular pattern (PAMP) gene that stimulates the innate immune system, such as flagellin, which is recognized by Toll-like receptor TLRS on immune cells. In some embodiments, a second or third gene comprises a danger associated molecular pattern (DAMP) gene such as heat shock proteins, HSP70, HSP90, heat shock factor 1 (HSF1), HMGB1 or 5100 proteins. Both PAMPs and DAMPs function through activating receptors (e.g., advanced glycosylation end product-specific receptor (AGER/RAGE), TLRs, NOD1-like receptors (NLRs), RIG-I-like receptors (RLRs), and AIM2-like receptors (ALRs) to produce inflammatory and immune responses (Bartlett, et al., Molecular Cancer 12:103, 2013; Tang, et al., Immunol. Rev., 249, 158-175, 2012; Huang, et al., Ageing Res Rev. S1568-1637(14)00113-5, 2014; Li, et al. Seminars in Cancer Biology, 23: 380-390, 2013).

In some embodiments, the nucleic acid construct for treatment of cancer includes two chains, heavy and light chain of a monoclonal antibody or fragment thereof, such as a Fab fragment or single chain variable fragment (scFv) or bispecific antibody. Such antibodies or fragments target proteins involved in angiogenesis or tumor growth such as VEGF or EGFR or HER2, for example (Finlay and Almagro, Front Immunol. 3:342 (2012); Dubel and Reichert Handbook of Therapeutic Antibodies, 2nd Edition Wiley Blackwell ISBN: 978-3-527-32937-3, 2014; Strohl and Strohl, Therapeutic Antibody Engineering, 1st Edition, Woodhead Publishing ISBN:9781907568374, 2012; Spiess, et al., Molecular Immunology 67: 95-106, 2015). Additionally or alternatively, in some embodiments, non-antibody protein scaffolds such as ankyrin repeats, fibronectin domains or three-helix bundle from Z-domain of Protein A from S. aureus amongst others (Hey, et al., Trends in Biotechnology 23: 514-522, 2005; Weidle, et al., Cancer Genomics and Proteomics 10:155-168, 2013) may be expressed under the control of the PEG promoter to receptors or growth factors involved in growth or maintenance of the tumor. In some embodiments, the heavy and light chain of a monoclonal antibody or fragment thereof, such as a Fab fragment or single chain variable fragment (scFv) is provided in addition to a second or third therapeutic gene. In some embodiments, the heavy and light chain of a monoclonal antibody or fragment thereof, such as a Fab fragment or single chain variable fragment (scFv) is provided instead of an immune checkpoint inhibitor fusion (e.g., is provided as the second gene), or instead of a therapeutic gene (e.g., is provided as the third gene).

In some embodiments, the nucleic acid construct for treatment of cancer includes a molecule that induces apoptosis, such as death receptors (DRs, for example TNFR1, CD95, DR3, TRAIL-R1 (CD4), TRAIL-R2 (CD5), and DR6) or their ligands, such as TNF, Fas ligand (FasL), and TNF-related apoptosis-inducing ligand (TRAIL) (Mahmood and Shukla, Experimental Cell Research 316: 887-899, 2010), or p53, p63 or p73 or pro-apoptotic members of the Bcl-2 family such as Bax, Bak, and their subclass of BH-3 only proteins such as BAD, BID, BIM, Hrk, PUMA, BMF, and Noxa related molecules (Tseng, et al., Nat Commun. 6:6456, 2015; Pflaum, et al., Front Oncol. 4: 285, 2014). In some embodiments, the molecule that induces apoptosis is provided in addition to the reporter gene, the immune checkpoint inhibitor fusion and the therapeutic gene. In some embodiments, the molecule that induces apoptosis is provided instead of an immune checkpoint inhibitor fusion (e.g., is provided as the second gene), or instead of a therapeutic gene (e.g., is provided as the third gene).

In some embodiments, the cancer-specific promoter, first gene, second gene, and third gene are cloned into a CpG-free plasmid, such as, for example, the Invivogen pCpGfree vectors. In some embodiments, the cancer-specific promoter, first gene, second gene, and third gene are cloned into a nanoplasmid, such as, for example, the Nature Technology Corporation NTC9385R plasmid. In some embodiments, the nucleic acid construct comprises a minicircle. Any suitable CpG-free plasmid, nanoplasmid, minicircle, or other expression vector may be used as components of the nucleic acid construct. In some embodiments, the nucleic acid construct is modified to be CpG-free. In some embodiments the nucleic acid construct is formulated in to a nanoparticle.

In some embodiments, the nucleic acid construct comprises the components set forth in the Table 1 below.

TABLE 1
Nucleic Acid Constructs

| | Promoter | 1^{st} gene | 2^{nd} gene | 3^{rd} gene |
|---|---|---|---|---|
| 1 | PEG-3 | mIL-12 | | |
| 2 | PEG-3 | HSV1-TK (2A) | mIL-12 | |
| 3 | PEG-3 | SR39 (2A) | mIL-12 | |
| 4 | PEG-3 | hIL-12 | | |
| 5 | PEG-3 | HSV1-TK (2A) | hIL-12 | |
| 6 | PEG-3 | SR-39 (2A) | hIL-12 | |
| 7 | PEG-3 | mIL-2 (2A) | mIL-12 | |
| 8 | PEG-3 | HSV1-TK (2A) | mIL-2 (2A) | mIL-12 |
| 9 | PEG-3 | SR39 (2A) | mIL-2 (2A) | mIL-12 |
| 10 | PEG-3 | hIL-2 (2A) | mIL-12 | |
| 11 | PEG-3 | HSV1-TK (2A) | hIL-2 (2A) | mIL-12 |
| 12 | PEG-3 | SR39 (2A) | hIL-2 (2A) | mIL-12 |
| 13 | PEG-3 | hIL-12 (2A) | hIL-2 | |
| 14 | PEG-3 | hIL-24 | | |
| 15 | PEG-3 | HSV1-TK (2A) | hIL-24 | |
| 16 | PEG-3 | SR39 (2A) | hIL-24 | |
| 17 | PEG-3 | mGM-CSF | | |
| 18 | PEG-3 | HSV1-TK (2A) | mGM-CSF | |
| 19 | PEG-3 | SR39(2A) | mGM-CSF | |
| 20 | PEG-3 | hGM-CSF | | |
| 21 | PEG-3 | HSV-TK (2A) | hGM-CSF | |
| 22 | PEG-3 | SR39 (2A) | hGM-CSF | |
| 23 | PEG-3 | mIL-12 (2A) | hIL-15 (2A) | |
| 24 | PEG-3 | HSV-TK (2A) | mIL-12 (2A) | hIL-15 |
| 25 | PEG-3 | SR39 (2A) | mIL-12 (2A) | hIL-15 |
| 26 | PEG-3 | cytokine (2A) | checkpoint | |
| | | | inhibitor gene* | |
| 27 | PEG-3 | HSV-TK (2A) | cytokine (2A) | checkpoint |
| | | | | inhibitor gene* |
| 28 | PEG-3 | SR39 (2A) | cytokine (2A) | checkpoint |
| | | | | inhibitor gene* |
| 29 | PEG-3 | checkpoint | Cytokine | |
| | | inhibitor gene* | | |
| 30 | PEG-3 | One of (a)-(c)** | PD-1 Fc | |
| 31 | PEG-3 | HSV1-TK (2A) | One of (a)-(c)** | PD-1 Fc |
| 32 | PEG-3 | SR39 (2A) | One of (a)-(c)** | PD-1 Fc |
| 33 | PEG-3 | Cytokine | One of (a)-(c)** | |
| 34 | PEG-3 | One of (a)-(c)** | Cytokine | |
| 35 | PEG-3 | HSV1-TK (2A) | One of (a)-(c)** | Cytokine |
| 36 | PEG-3 | SR39 (2A) | One of (a)-(c)** | Cytokine |

*where the checkpoint inhibitor gene encodes molecules such as an anti-CTLA4, anti-PD1, anti-PD-L1 monoclonal or PD-1 Fc fusion (2A)
**where (a) is a heavy and light chain of an antibody, or fragments thereof, (b) is a molecule that induces apoptosis, and (c) is a molecular pattern gene (2A)

The vector compositions (preparations) of the present technology are typically administered systemically, although this need not always be the case, as localized administration (e.g. intratumoral, or into an external orifice such as the vagina, the nasopharyngeal region, the mouth; or into an internal cavity such as the thoracic cavity, the cranial cavity, the abdominal cavity, the spinal cavity, etc.) is not excluded. For systemic distribution of the vector, routes of administration include but are not limited to: intravenous, by injection, transdermal, via inhalation or intranasally, or via injection or intravenous administration of a cationic polymer-based vehicle (e.g. in vivo-jetPEI®), liposomal delivery, which when combined with targeting moieties will permit enhanced delivery. The ultrasound-targeted microbubble-destruction technique (UTMD) may also be used to deliver therapeutic agents (Dash, et al. Proc Natl Acad Sci USA. 108:8785-90, 2011); hydroxyapatite-chitosan nanocomposites (Venkatesan, et al. Biomaterials. 32:3794-806, 2011); and others (Dash, et al. Discov Med. 11:46-56, 2011); etc. Any method that is known to those of skill in the art, and which is commensurate with the type of construct that is employed, may be utilized. In addition, the compositions may be administered in conjunction with other treatment modalities known in the art, such as various chemotherapeutic agents such as Pt drugs, substances that boost the immune system, antibiotic agents, and the like; or with other detection or imaging methods (e.g. to confirm or provide improved or more detailed imaging, e.g. in conjunction with mammograms, X-rays, Pap smears, prostate specific antigen (PSA) tests, etc.

In some embodiments, the nucleic acid will be formulated into nanoparticles using the cationic polymer linear PEI at N/P ratio of 4 or 6. In some embodiments the nanoparticles are lyophilized in a cryoprotectant sugar solution, such as 9.5% Trehalose.

Those of skill in the art will recognize that the amount of a construct or vector that is administered will vary from patient to patient, and possibly from administration to administration for the same patient, depending on a variety of factors, including but not limited to: weight, age, gender, overall state of health, the particular disease being treated, and concomitant treatment, thus the amount and frequency of administration is best established by a health care professional such as a physician. Typically, optimal or effective tumor-inhibiting or tumor-killing amounts are established e.g. during animal trials and during standard clinical trials. Those of skill in the art are familiar with conversion of doses e.g. from a mouse to a human, which is generally done according to body surface area, as described by Freireich, et al. (Cancer Chemother Rep 50:219-244, 1996); and see Tables 2 and 3 below, which are taken from the website located at dtp,nci.nih.gov.

TABLE 2
Conversion factors in mg/kg

| | Mouse wt. | Rat wt | Monkey wt | Dog wt | Human wt |
|---|---|---|---|---|---|
| | 20 g | 150 g | 3 kg | 8 kg | 60 kg |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | 1⅔ | 1 | ½ |
| Man | 12 | 7 | 3 | 2 | 1 |

For example, given a dose of 50 mg/kg in the mouse, an appropriate dose in a monkey would be 50 mg/kg×¼=13 mg/kg/; or similarly, a dose of about 1.2 mg/kg in the mouse is about 0.1 mg/kg for a human.

TABLE 3
Representative Surface Area to Weight Ratios

| | Body | Surface | Km |
|---|---|---|---|
| Species | Weight (kg) | Area (sq. m.) | factor |
| Mouse | 0.02 | 0.0066 | 3.0 |
| Rat | 0.15 | 0.025 | 5.9 |
| Monkey | 3.0 | 0.24 | 12 |
| Dog | 8.0 | 0.4 | 20 |
| Human, child | 20 | 0.8 | 25 |
| Human, adult | 60 | 1.6 | 37 |

To express the dose as the equivalent mg/sq.m. dose, multiply the dose by the appropriate factor. In adult humans, 100 mg/kg is equivalent to 100 mg/kg×37 kg/sq.m.=3700 mg/sq.m.

In general, for treatment methods, the amount of a vector such as a plasmid will be in the range of from about 0.01 to about 5 mg/kg or from about 0.05 to about 1 mg/kg (e.g. about 0.3 mg/kg) of plasmid, and from about 10⁵ to about 10²⁰ infectious units (IUs), or from about 10⁸ to about 10¹³ IUs for a viral-based vector.

Typically, cancer treatment requires repeated administrations of the compositions. For example, administration may be daily or every few days, (e.g. every 2, 3, 4, 5, or 6 days), or weekly, bi-weekly, or every 3-4 weeks, or monthly, or any combination of these, or alternating patterns of these. For example, a “round” of treatment (e.g. administration one a week for a month) may be followed by a period of no administration for a month, and then followed by a second round of weekly administration for a month, and so on, for any suitable time periods, as required to optimally treat the patient.

The subjects or patients to whom the compositions of the present technology are administered are typically mammals, frequently humans, but this need not always be the case. Veterinary applications are also contemplated, such as dogs, for example.

The constructs and methods of the present technology are not specific for any one type of cancer. As will be understood by one of skill in the art, “cancer” refers to malignant neoplasms in which cells divide and grow uncontrollably, forming malignant tumors, and invade nearby parts of the body. Cancer may also spread or metastasize to more distant parts of the body through the lymphatic system or bloodstream. The constructs and methods of the present technology may be employed to image, diagnose, treat, monitor, etc. any type of cancer, tumor, neoplastic or tumor cells including but not limited to: osteosarcoma, ovarian carcinoma, breast carcinoma, melanoma, hepatocarcinoma, lung cancer, brain cancer, colorectal cancer, hematopoietic cell, prostate cancer, cervical carcinoma, retinoblastoma, esophageal carcinoma, bladder cancer, neuroblastoma, renal cancer, gastric cancer, pancreatic cancer, and others.

In addition, the present technology may also be applied to the treatment of benign tumors, which are generally recognized as not invading nearby tissue or metastasizing. Illustrative benign tumors include but are not limited to moles, uterine fibroids, etc.

### Combinatorial Therapies

The constructs and methods of the present technology may be used in combination with one or more additional cancer treatments as known in the art. For example, treatments comprising the administration of molecules that inhibit pathways such as BRAF/MEK, AKT-PI3K-mTOR, Wnt-β-catenin, EGF/EGFR, chemotherapy agents, radiotherapy or inhibitors of checkpoint molecules, angiogenesis or indoleamine 2,3-dioxygenase, or inhibitors of FOXP3 for example (Lozano, et al., Oncotarget, 8, 71709-71724, 2017; immunotherapy combinations reviewed by Ott, et al., Journal for ImmunoTherapy of Cancer, 5:16, 2017; interleukin 12 combinations reviewed by Lasek and Jakóbisiak, Interleukin 12: Antitumor Activity and Immunotherapeutic Potential in Oncology, SpringerBriefs in Immunology, Springer International Publishing AG ISBN 978-3-319-46906-5, 2016).

Methods and compositions of the present technology and one or more additional cancer treatments may be administered to subject in need thereof separately, simultaneously, or sequentially.

### EXAMPLES

### Example 1: Cloning of Therapeutic Constructs

Removal of CpG sites from a therapeutic plasmid is not an obvious requirement in cancer therapeutics. It has been reported that formulated plasmids containing IL-12 and LacZ (4.5% and 7.4% CpG, respectively) expressed from a CMV promoter and delivered using linear PEI had a similar response to each other in a model of LLC (LL/2) tumors in C57BL/6 mice, therefore demonstrating the immune-stimulatory effect of CpG sites irrespective of payload (Rodrigo-Garzón et al., Cancer Gene Therapy, 17; 20-27, 2010). In that study, the reduction of CpG sites was not investigated and it was concluded that in the case of a lung cancer model using LLC (LL/2) cells, the antitumoral activity is mainly driven by the activation of the innate immune system by the CpG motifs. This activation was not specifically directed at the tumors as the particles were not targeted nor was the gene expression selective for cancerous cells. Therefore, expression from the plasmid payload could occur outside of the region of the tumor, potentially introducing toxicity associated with high systemic levels of cytokine.

It is the intention of the work described within this current application to limit the biological effects to the expressed payload produced within the tumor microenvironment, i.e., to the proteins expressed under the control of the PEG-3 promoter, which is activated within tumor cells, rather than to innate immunity driven solely by the CpG content of the DNA encapsulated within the particles. Hence, CpG-free ORFs (open reading frames) were designed and cloned into the plasmid and subsequently formulated into nanoparticles.

All therapeutic constructs were modified to remove CpG motifs and codon optimized. For all expression cassettes, the termini of the sequences were modified to include a 5′ restriction enzyme site compatible with the plasmid/PEG-3 promoter sequence and a stop codon followed by a NheI site at the 3′ end, to insert into CpG free expression plasmids, such as pCpGfree-N-mcs (Invivogen, San Diego, Calif., US), or other CpG free plasmids, in which the PEG-3 promoter was cloned in place of the mCMV enhancer and EF1 promoter.

Cytokines were cloned in isolation or in combination with additional gene payloads such as CpG-free HSV-1 TK (TK) (SEQ ID NO: 1) or modified CpG-free thymidine kinase (SR39) (SEQ ID NO: 2) expressed from a single PEG-3 promoter. These cytokines include: murine IL-12 (mIL12); TK and murine IL-12 (TK-mIL12); human IL-12 (hIL12); TK and human IL-12 (TK-hIL12); murine IL-2 and murine IL-12 (mIL2-mIL12); TK and murine IL-2 and murine IL-12 (TK-mIL2-mIL12); TK and human IL-2 and murine IL-12 (TK-hIL2-mIL12); human IL-12 and human IL-2 (hIL12-hIL2); human IL-24 (hIL24); TK and murine GM-CSF (TK-mGM-CSF); TK and human GM-CSF (TK-hGM-CSF); mIL-12 and hIL-15 (mIL12-hIL15); TK and mIL-12 and hIL-15 (TK-mIL12-hIL15); TK and murine IL-12 and flagellin (FliC) (TK-mIL12-Flag).

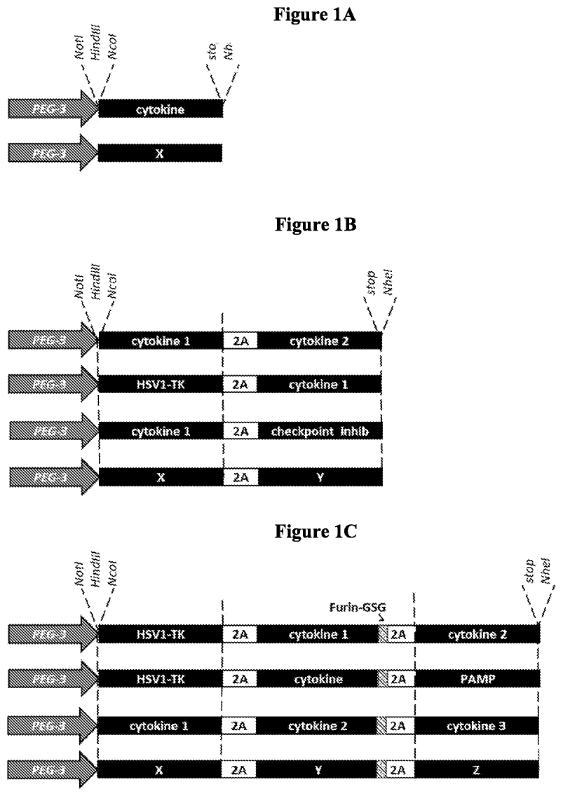

Cytokine sequences: The sequences of human IL-2 (Genbank S77834.1), murine IL-2 (NCBI NM_008366.3); human single chain IL-12 (Human Gene Therapy 1997, 8, 1125-1135), murine single chain IL-12, human IL-15 (Genbank AF031167.1), human MDA 7/IL-24 (NCBI NM_006850.3), human GM-CSF (Genbank M11220.1), murine GM-CSF (GenBank EU366957.1) were analyzed for CpG motifs and rare codons were mutated such that the protein coding sequence was unaffected. These modified sequences were human IL-2 (1 CpG site mutated—SEQ ID NO: 3), murine IL-2 (SEQ ID NO: 4), human single chain IL-12 (30 CpG sites mutated—SEQ ID NO: 5), murine single chain IL-12 (45 CpG sites mutated—SEQ ID NO: 6), human IL-15 containing an IL-2 secretion signal placed upstream of the IL-15 sequence for secretion (3 CpG sites mutated—SEQ ID NO: 7), human MDA 7/IL-24 (9 CpG sites mutated—SEQ ID NO: 8), and human GM-CSF (10 CpG sites mutated—SEQ ID NO: 9), murine GM-CSF (12 CpG sites mutated—SEQ ID NO: 10).

Where the gene ORFs were cloned as a single expression cassette, the gene's coding regions were made with one of the sites NotI, HindIII or NcoI at the 5′ end to fit the restriction endonuclease sites of the PEG-3 promoter and a stop codon and NheI site at the 3′ terminus for cloning into the plasmid (FIGS. 1A-1B). Where there were two ORFs in the cassette, the first ORF was cloned so that it was made with one of the sites NotI, HindIII or NcoI at the 5′ end, a 3′ BamHI or a type IIS restriction site such as Esp3I (Esp3I is a type IIS restriction enzyme that cleaves DNA outside of its recognition site and can be used for “scarless” cloning so that no extraneous sequence is introduced) and no stop codon. The second ORF contained a 5′ BamHI site or a type IIS restriction site such as Esp3I, followed by a 2A ribosome skipping sequence in frame with the gene sequence, a 3′ stop codon and 3′ NheI site. Optionally, a furin cleavage site (RRKR) and GSG linker could be placed 5′ to the 2A site where post translational removal of the 2A site is required. Where there were 3 genes in a cassette, the first ORF was made with one of the sites NotI, HindIII or NcoI at the 5′ end and a 3′ BamHI site or type II S restriction site and lacking a stop codon. The second gene contained a 5′ BamHI (followed by a 2A sequence) or type IIS site and a 3′ Esp3I site (or another appropriate type IIs restriction site) and did not contain a stop codon. The 3′ Esp3I site in the second gene was preceded by a furin cleavage site (RRKR) and GSG linker and a 2A ribosome skipping sequence. The third gene was cloned using a 5′ Esp3I site, a 3′ stop codon and 3′ NheI site. Additional genes can be cloned to the construct using type IIS restriction enzymes and expressed as discrete proteins using additional furin cleavage signals, GSG linkers and 2A ribosome skipping sequences in between the genes. The 3′-end of such expression cassettes would encode a stop codon and a NheI site for cloning into the modified pCpGfree-PEG plasmid upstream of the polyA sequence.

PD-1 Fc: The extracellular domain (ECD) of human PD-1 (UniProt Q15116 residues 21-170) was used as a sequence for the design of PD-1-Fc. This sequence was modified to optimize codon usage and remove CpG sites. The PD-1 sequence, to be used in the fusion, encompassed residues 25-170 fused to a signal sequence from human IgG heavy chain 5′ to the PD-1 coding region (for secretion from the cells). As an example of cloning, a 5′ BamHI restriction endonuclease and a P2A ribosome skipping sequence are placed 5′ to the signal sequence. The BamHI site is used for ligation of a first gene containing a 3′ BamHI site, for example, to the P2A-signal sequence-PD1ECD cassette following digestion with BamHI of both products, purification and ligation with T4 ligase. In the human PD-1 sequence, Cys 73 is mutated to Ser in order to assist expression and folding (Cheng et al. J. Biol. Chem. 288: 11771-11785, 2013). At the C-terminus of the PD-1 sequence, the Fc sequence (hinge region/CH2/CH3 domains) of IgG4 heavy chain are joined. In this example, human IgG4 is used so that there is reduced binding to Fcγ receptors. Other IgG isotypes can be used such as IgG1 from human or from other species, such as mouse IgG2a. Mutations within the hinge region (at position 228 (serine to proline) and at 235 (leucine to glutamic acid) (EU numbering)) of the heavy chain are introduced to stabilize the hinge and reduced binding to FcγRI, respectively. The IgG4 sequence 216-447 (EU numbering) is followed at the 3′ end by a furin cleavage site (RRKR) and GSG linker and T2A ribosome skipping sequence and a Esp3I site to enable “scarless” cloning of the third protein onto the P2A-signal sequence-PD1ECD-Fc-FurinGCGT2A fragment (50 CpG sites removed—SEQ ID NO: 11).

The known TLRS stimulatory epitopes of flagellin (FliC) from Salmonella typhimurium (Genbank D13689.1) (76 CpG sites removed—SEQ ID NO: 12) were synthesized as codon optimized and CpG-free sequences. The primary sequences of these regions were not altered to remove the potential glycosylation sites, although this may be a consideration as native FliC is not glycosylated. Flagellin DNA sequence encoding amino acids 1-191 and 336-495 were synthesized (although full-length protein can be used) with a 5′ Esp3I site and a 3′ stop codon and NheI site for cloning downstream of a first and second gene.

Monoclonal, bispecific or fragments of antibodies can be expressed alone or within a construct expressing murine or human IL-12, for example they can be cloned downstream of the IL-12 sequence, a furin cleavage site a BamHI cloning site and a 2A ribosomal skipping sequence. CpG-free constructs were designed through reverse translation of the peptide sequence using a codon optimized CpG-free human biased genetic code matrix. The expression cassette is exemplified for monoclonal antibodies in an expression cassette with IL-12 such as hIL12-ipilimumab (Drug Bank DB06186) (SEQ ID NO: 13), hIL12-pembrolizumab (Drug Bank DB09037) (SEQ ID NO: 14), hIL12-nivolumab (Drug Bank DB09035) (SEQ ID NO: 15), hIL12-bevacizumab (Drug Bank DB00112) (SEQ ID NO: 16), hIL12-durvalumab (Drug Bank DB11714) (SEQ ID NO: 21), hIL12-atezolizumab (Drug Bank DB11595) (SEQ ID NO: 22). This is also exemplified for a bispecific blinatumomab, hIL12-blinatumomab (Drug Bank DB09052) (SEQ ID NO: 17) and Fab ranibizumab (Drug Bank DB01270) (hIL12-ranibizumab, SEQ ID NO: 18) and anti-murine PD-1 monoclonal, iTME (WO2016/170039) (mIL12-iTME SEQ ID NO: 19).

### Example 2: In Vitro Expression Analysis

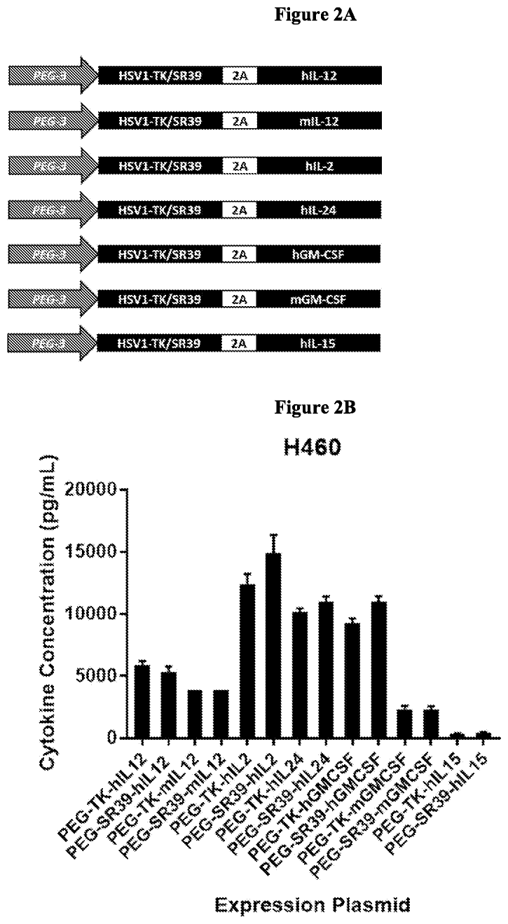

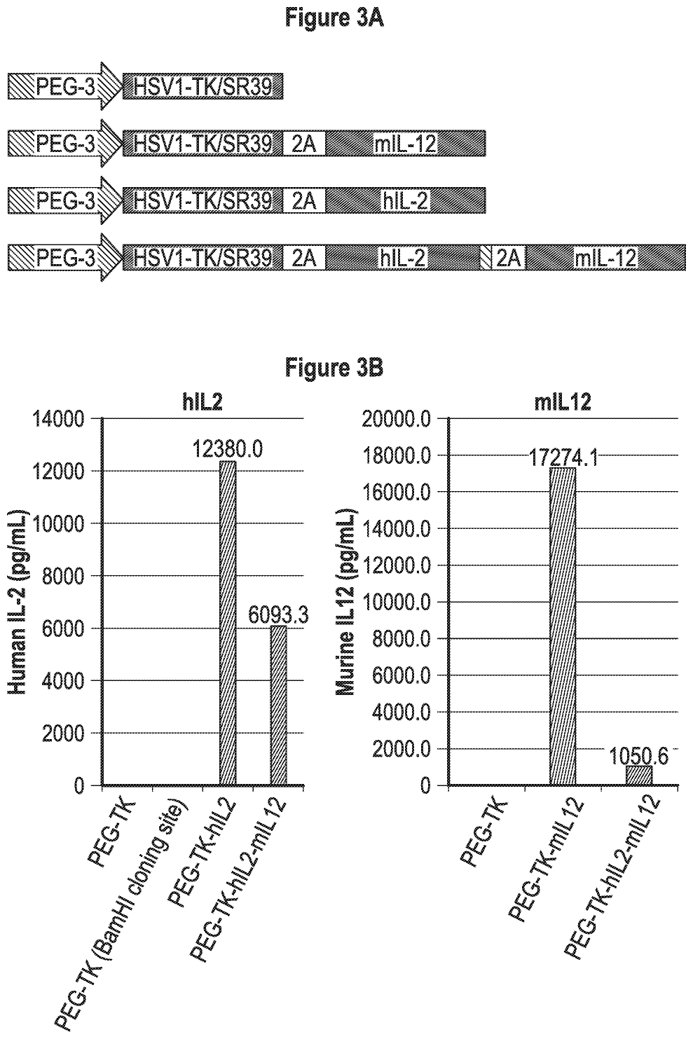

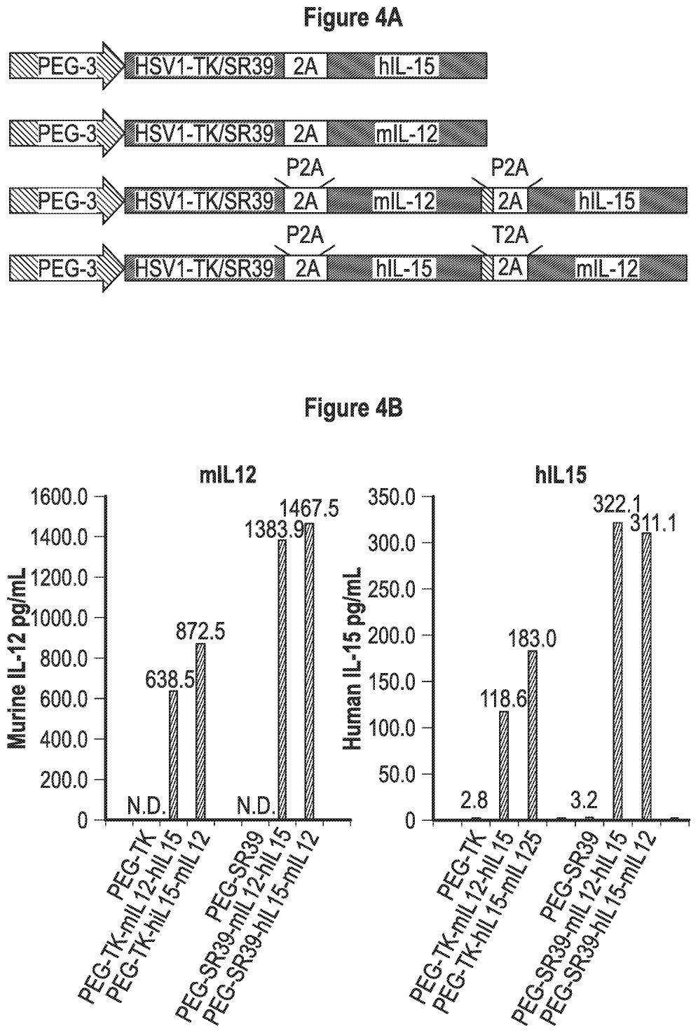

Constructs were transfected into cultured cancer cells, such as human lung cancer cell lines H460 (ATCC® HTB-177™) or H1975 (ATCC® CRL-5908™) or murine lung cancer cell line LL/2 (Perkin Elmer, Watham, Mass.), and tested for expression of the individual proteins by ELISA. Plasmids were formulated with jetPRIME (Polyplus Transfection, Illkirch, FRANCE) according to the manufacturer's instructions. For example, LL/2 cells were plated at a density of 10e5 cells/well in a 12 well plate in DMEM. 1 μg of plasmid was diluted into 25 μL of serum free media and vortexed gently. 4 μL PEIpro was added into 25 μL of serum free media and the PEIpro solution was added to the DNA solution and vortexed gently, followed by 15 min incubation at room temperature. The cells were incubated at 37° C. in 5% CO₂ for 48 hours. Culture supernatant was then removed and stored at −20° C. until testing by ELISA using the relevant anti-cytokine Quantikine ELISA kit (R & D Systems, Minnesota, USA) according to the manufacturer's instructions. Dilutions of the culture supernatants were made in duplicate and quantitation of cytokine expression was measured against standard curves of known standards (FIGS. 2A-2B, 3A-3B, 4A-4B).

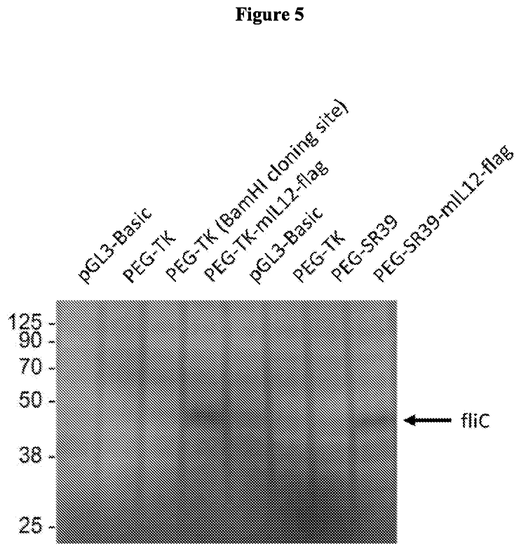

FliC expression was monitored by Western blot analysis in the following manner. Cells were lysed by adding T-per® Tissue Protein Extraction Reagent (#78510, Thermo Fisher, Waltham, Mass., USA) and incubating in ice for 15 min. After clarifying by centrifugation, the total amount of protein was determined by Coomassie (Bradford) Protein assay. A total of 30 μg of cell extract (per well) were loaded on to SDS-PAGE gel. After electrophoresis, proteins were transferred to a polyvinylidenefluoride membrane (Bio-Rad) using a Trans-Blot® TURBO transfer (Bio-Rad). The membrane was blocked with 5% BSA in TBS-T (10 mM Tris-Cl pH 8.0, 150 mM NaCl, 0.01% Tween-20) for 1 hour at room temperature and incubated overnight with 1:1000 dilution of anti-FliC primary antibody (#629701, BioLegend, San Diego, Calif., USA) at 4° C. in the same buffer. After washing the membrane four times with TBS-T for 10 minutes, the membrane was incubated with goat anti-mouse HRP secondary antibody (#31430, Thermo Fisher, Waltham, Mass., USA) diluted 1:10,000 in 5% BSA TBS-T for 1 h at room temperature followed by four washes with TBS-T for 10 minutes. The membrane was visualized by Clarity™ Western ECL kit (BIO-RAD) and ChemiDoc™ XRS+imaging system (BIO-RAD) (FIG. 5).

### Example 3: Thymidine Kinase Activity

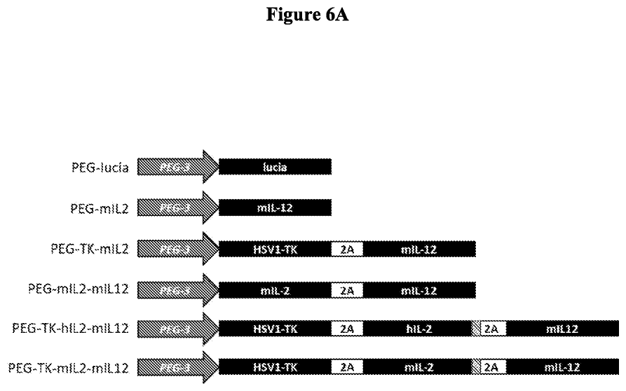

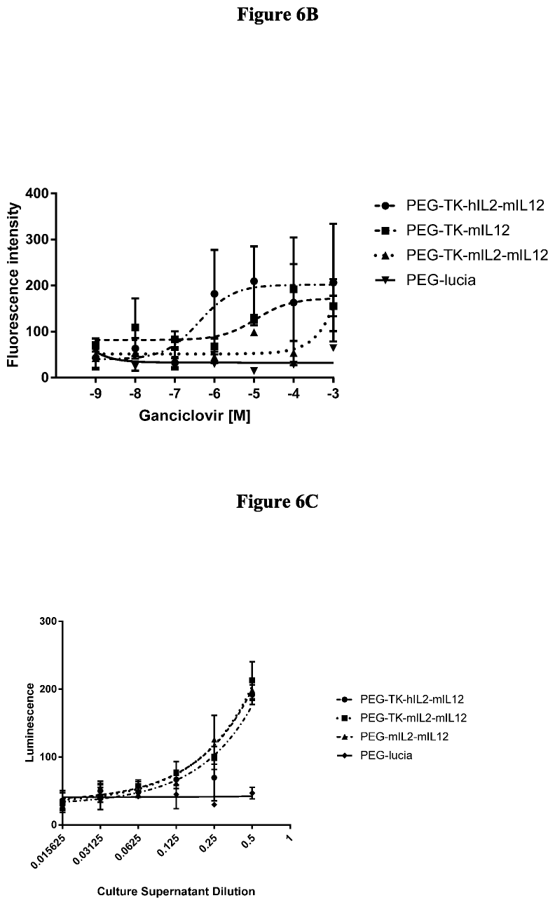

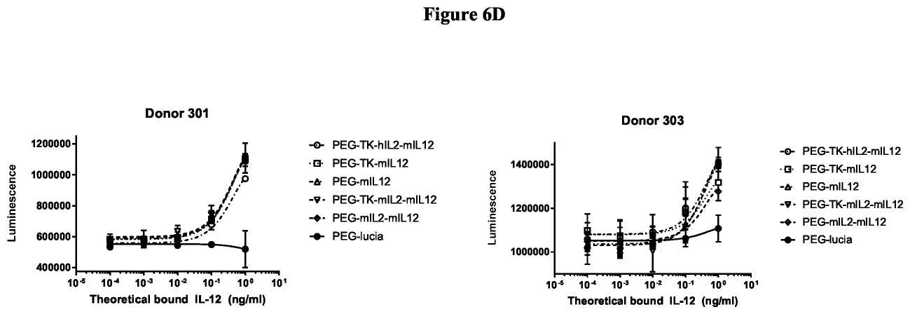

1.5 μg of PEG-TK-hIL2-mIL12, PEG-TK-mIL12, PEG-mIL12 or PEG-lucia plasmid (FIG. 6A) were diluted in 75 μL of pre-warmed OptiMEM medium and gently vortexed. 12 μL of PEIpro reagent was diluted into 75 μL of OptiMEM. The PEIpro solution was then added to the DNA solution and vortexed gently. The DNA/PEIpro solution was incubated for 15 min at room temperature. 2.5 μL of the DNA/PEIpro solution was added to the 96-well plate. LL/2-Red-FLuc cells (Perkin Elmer, Waltham, Mass.) were cultured in a T175 flask until 60-70% confluent. The cell monolayer was briefly washed with 20 mL PBS, trypsinized with 3 mL of trypsin/EDTA for 3 min and 7 mL of media was added once the cells were removed from the surface. The suspension was transferred to a 15 mL Falcon tube and centrifuged at 200 g for 5 min. The supernatant was removed and the cell pellet was resuspended in 3 mL of fresh media. Cells were plated at 1,000 or 5,000 (assay dependent) cells/well in a 96-well plate in 100 μL per well of complete DMEM media. Plates were transferred to a 37° C./5% CO₂ incubator and allowed to grow for 24 hours prior to compound treatment. A 100 mM stock was prepared in DMSO and used to prepare a 10-fold dilution series from 1000 mM to 0.01 μM in DMSO. The media containing transfection reagent were removed from the transfection plate and replaced with 50 μL/well of respective ganciclovir concentration (triplicate wells for each concentration). The plate was incubated for 48 hours at 37° C. CellTox™ Green Cytotoxicity reagent (Promega, Madison, Wis.) was made up to 2× with assay buffer and 50 μL of reagent was added to each well of the 96-well plate with the cells incubated with ganciclovir. The plate was incubated for 15 min at room temperature, protected from light and the green fluorescence was read at 485 nm (excitation) and 520 nm (emission). When the cells were treated with escalating doses of ganciclovir, there was a clear increase in fluorescence intensity (which directly correlates to cytotoxicity of the cells) in the particles formulated with PEG-TK-hIL2-mIL12, PEG-TK-mIL2-mIL12, and PEG-TK-mIL12 that expressed HSV1-TK, but not in nanoparticles formulated with PEG-lucia plasmid that did not express HSV1-TK (FIG. 6B).

### Example 4: Functional Analysis of Expressed IL-2 and mIL-12 In Vitro

The CTLL-2 cell line (ECACC 93042610) is a cytotoxic T cell line of mouse origin derived from C57BL/6 inbred mice (H-2b) and is dependent upon stimulation from IL-2 for survival and growth. In this assay, proliferation was induced by IL-2 expressed in the culture media of a LL/2 cell line transfected with nanoparticles containing engineered plasmids of the PEG-3 promoter and expressing murine IL-2 or human IL-2 in a cassette with mIL-12 (mIL2-mIL12:). Both human and murine IL-2 can act on CTLL2 cells and mIL-12 has also been shown to have a proliferative effect in the presence of IL-2. As a positive control, lyophilised recombinant hIL-2 (rhIL-2) was reconstituted to 100 μg/mL in 100 mM sterile acetic acid containing 0.1% BSA. Stock rhIL-2 was diluted down to 500 ng/mL in RPMI 1640 without T-Stim, which was used to prepare a 2-fold dilution series from 20 ng/mL to 0.163 ng/mL in a 96-well intermediate plate in a final volume of 100 μL/well. 50 μL of each dilution was transferred into the final cell proliferation plate. A 2-fold dilution series from 1:2 to 1:32 for cell culture supernatants was prepared in RPMI 1640 without T-Stim (125 μL:125 μL media). 50 μL of each dilution was transferred into the final cell proliferation plate.

CTLL2 cells that had been maintained at 2×10e5 cells/mL in complete RPMI media (containing T-Stim) were collected and centrifuged at 400 g for 5 min. Cells were re-suspended in 20 mL RPMI media containing all additional supplements except T-Stim and cultured for a further 24 hours at 37° C. in 5% CO₂. Cells were then plated at 4×10e4 cells/well in a 96-well plate in 50 μL of RPMI media without T-Stim on the final cell proliferation plate. In order to assay proliferation, 100 μL of CellTiter-Glo® Reagent (Luminescent Cell Viability Assay, Promega Corp., Madison, Wis.) was added to the cells in line with the manufacturer's guidelines for the CellTiter-Glo® Reagent. Cells were incubated at room temperature (with shaking at 500 rpm) for 15 minutes and the luminescence was recorded on a luminometer and quantified using a standard curve as per manufacturer's instructions. The results show that undiluted culture supernatant in LL/2 cell transfected with PEG-mIL2-mIL12 (SEQ ID NO: 20), PEG-TK-mIL2-mIL12, and PEG-TK-hIL2-mIL12 nanoparticles caused proliferation of CTLL2 cells, which demonstrates expression of active IL-2 and mIL-12. (FIG. 6C).

### Example 5: Functional Analysis of Expressed IL-12 In Vitro

Peripheral blood mononuclear cells (PBMCs) were isolated from whole blood samples by Ficoll Hypaque gradient centrifugation. 10e7 PBMCs were added to a total of 20 mL supplemented medium in a 75 cm² culture flask. 20 μL of 10 mg/mL phytohemagglutinin (PHA) (200 μg PHA) was added and the flask was incubated for 3 days at 37° C. in 5% CO₂. 20 mL of supplemented media was added and then gently mixed by shaking. 20 mL of the contents were then transferred to a clean 75 cm² culture flask and human recombinant IL-2 was added to 50 U/mL and further incubated for 24 hours at 37° C. in 5% CO₂. PBMCs were diluted to 2×10e5 cells/mL for use in the assay.

A 96-well plate was coated with 5 μg/mL mouse anti-IL-12 antibody in NaCO₃ or PBS buffer and incubated at 4° C. overnight. Plates were washed with buffer and then blocked with 1% BSA/PBS for 1 hour at room temperature. Serial dilutions of mIL-12 reference compound (5 ng/mL to 0.008 ng/mL) and cell supernatant (containing expressed mIL-12) were made and 100 μL of reference or test sample dilutions were added to the wells, followed by incubation for 2.5 to 3 hours at room temperature. The plate was washed with PBS buffer and 100 μL PHA stimulated PBMC cells were added (2×10e4 cells/well). The cells were incubated for 7 days at 37° C. in 5% CO₂. Cell proliferation was detected using CellTiter-Glo® Reagent according to the manufacturer's instructions. Cell culture supernatants from LL/2 cells that were transfected with nanoparticles expressing mIL-12 showed a proliferative response from PBMCs isolated from two human donors (FIG. 6D).

### Example 6: Activity of PEG-3 Plasmid Formulated Nanoparticles in a Syngeneic In Vivo Model of Mouse Primary Lung Cancer (Orthotopic LL/2 in C57BL/6 Mice)

Tumor cell culture and inoculation—LL/2-Red-FLuc mouse lung tumor cells (Perkin Elmer, Waltham, Mass., USA) were cultured in MEM supplemented with 10% FBS, 1% GlutaMAX™ and 1% penicillin-streptomycin, and grown at 37° C. in a humidified cell culture incubator supplied with 5% CO₂ (materials supplied by Invitrogen, Carlsbad, Calif., USA). The cells were harvested (Passage 2) by trypsinization, washed twice in HBSS and counted (using trypan blue exclusion). The final cell density was adjusted with HBSS:Matrigel™ (BD Biosciences, East Rutherford, N.J., USA) (1:1, v/v) to 2×10e6 cells/mL. Female C57BL/6 (Envigo, Indianapolis, Ind., USA) mice were inoculated while under intraperitoneally injected anesthesia (Ketamine (14 mg/mL)/Xylazine (1.2 mg/mL)) (Clipper Distributing Company, St Joseph, Mo., USA). The skin at the injection site was liberally swabbed with alcohol and 20 μL aliquot of cell suspension containing 4×10e4 LL/2-Red-FLuc cells were injected into the pleura. Mice were administered a 200 μL bolus dose of Buprenex (Buprenorphine HCl, 0.01 mg/mL) (Hospira, Inc, Lake Forest, Ill., USA) subcutaneously for pain relief at the time of surgery and the following day. The presence of lung tumors was confirmed based on a positive luminescence signal in the thoracic region of whole at Study Day 5. Animals (with positive luminescent signal) were randomized using a matched pair distribution method, based on body weight, into groups of 10, five days post-inoculation (Study Day 5). Procedures involving the care and use of animals in the study were reviewed and approved by the Pennsylvania State College of Medicine Institutional Animal Care and Use Committee prior to conduct. During the study, the care and use of animals was conducted in accordance with the principles outlined in the Guide for the Care and Use of Laboratory Animals, 8th Edition, 2011 (National Research Council).

Monitoring—Mortality and checks for clinical signs were performed once daily in the morning during the study. Body weights were recorded for all animals on Study Day 5 and then at least twice weekly, including the termination day. Whole body imaging was performed at inoculation (Study Day 0) and then all remaining animals on Study Days 5, 9, 13 and at termination.

Formulation of nanoparticles for in vivo use. Nanoparticles comprising of the plasmid and a linear PEI polymer (in vivo-jetPEI®, Polyplus Transfection, Illkirch, France) were prepared under high pressure using a confined impinged jet (CIJ) device. In this device, the streams are impinged in the confined chamber at high Reynolds number, thereby causing the water-soluble polycationic polymers and the water-soluble polyanionic nucleic acid to undergo a polyelectrolyte complexation process that continuously generates nanoparticles. The CIJ device and all the fittings were autoclaved on a dry cycle prior to use. A working solution of in vivo-jetPE10 was made in 9.5% Trehalose and combined under pressure with a stock solution of plasmid in 9.5% Trehalose (according to Patent Application US 2017/0042829). PEG-3 plasmids containing CpG-free genes for mIL-12, TK-hIL2-mIL12 (PEG-mIL-12, PEG-TK-hIL12-mIL12, respectively) or lucia luciferase (Invivogen, San Diego, Calif., USA) (PEG-lucia) were formulated at a N/P=6 ratio followed by lyophilization in 0.05 mg (DNA) aliquots. 0.05 mg (DNA content) of each formulated plasmid, PEG-TK-hIL2-mIL12, plasmid PEG-mIL12 or PEG-lucia control, was reconstituted in 250 μL of nuclease-free water on the day of dosing. Formulated test articles were stored at 4° C. until use on the same day. 9.5% Trehalose buffer was used as a vehicle control. 0.04 mg of each plasmid formulation were administered via intravenous injection (i.v.) in a fixed volume of 200 μL/animal on Study Days 5, 9, 13, 17, and 21.

Imaging—In vivo whole-body luminescence imaging was performed on all animals at inoculation (Study Day 0) and then on all remaining animals on Study Days 5, 9, 13, and at termination using the Perkin Elmer IVIS.Lumina XR imaging system. Animals were administered 150 mg/kg D-luciferin (15 mg/mL solution prepared in PBS) via intraperitoneal injection and were imaged 5-10 minutes later while under isoflurane anesthesia. Animals were allowed to recover from anesthesia prior to dosing. Luminescence signal was measured in the region of interest (thoracic region) and images were captured. Images were analyzed using Living Image 4.4 (Caliper Life Sciences, Hopkinton, Mass., USA).

Termination procedure—All animals were anesthetized for blood collection and euthanized by exsanguination via terminal cardiac bleed by approved standard procedures. The study was terminated on Study Day 23 as the majority of animals had reached the ethical end-point of body weight loss or adverse clinical observations or had died from unknown causes.

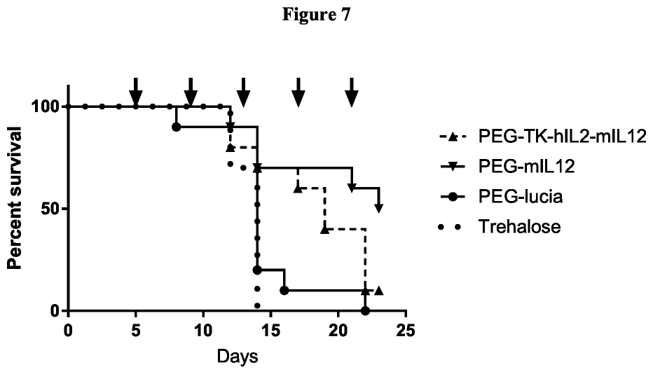

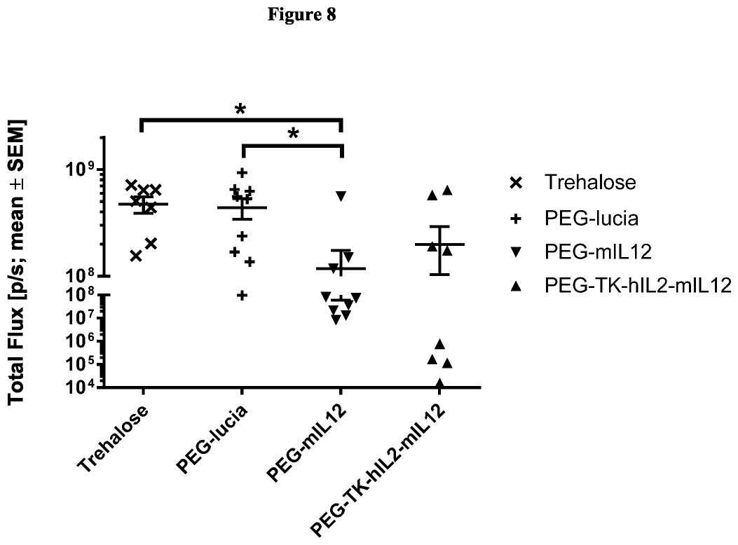

Results—The study was terminated at Day 23 when only six animals remained alive, one treated with plasmid PEG-TK-hIL2-mIL12 and five treated with plasmid PEG-mIL12. Survival time was significantly (p≤0.05) prolonged in animals receiving plasmid PEG-TK-hIL2-mIL12 and PEG-mIL12 compared with vehicle control (9.5% Trehalose), as shown by Kaplan Meier Analysis (FIG. 7). Median survival times were 19 days for PEG-TK-hIL2-mIL12, 23 days for PEG-mIL12 and 14 days for PEG-lucia and the Trehalose groups. Luminescence in vivo imaging on Study Day 13 (after two doses had been administered) showed plasmid PEG-mIL12 to significantly reduce (p≤0.05; Dunnett's Multiple Comparisons Test) the growth of the lung tumors, as indicated by reduced luminescence in the lung region (corresponding to less LL/2-Red-FLuc tumor cells), compared with 9.5% Trehalose control (FIG. 8).

Therefore, two formulations of nanoparticles made in the CIJ device at N/P=6, the single payload cassette (mIL-12) and the three payload cassette (TK-IL2-IL12), improved survival of mice that had been orthotopically inoculated with tumors in the lungs (FIG. 7). In addition, PEG mIL-12 nanoparticles showed a significant reduction in the luminescence of the tumor cells in vivo at Day 13 post inoculation, which is indicative of reduced tumor growth in the lungs (FIG. 8). Accordingly, these results demonstrate that the formulations of the present technology are useful in methods for treating cancer in a subject in need thereof.

### Example 7: Syngeneic Model of Mouse Primary Lung Cancer LL/2

In a second experiment, animals in each group received treatment with either 9.5% Trehalose Control (in a fixed volume of 200 μL/animal) or one of the plasmid-in vivo-jetPEI® formulations (N/P=6) (plasmid PEG-mIL12, plasmid PEG-TK-mGMCSF, plasmid PEG-TK-hIL15-mIL12, plasmid PEG-TK-mIL12-flag and PEG-lucia) each at 2 mg/kg in a dosing volume of 10 mL/kg. All treatments were administered via intravenous injection (i.v.) on Study Days 5, 9, 13, 17, and 21. Methods were as described in Example 6 above for animal treatment and imaging.

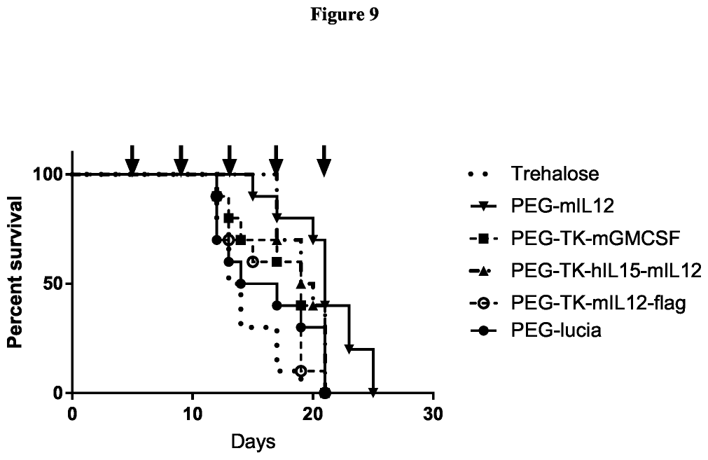

Results—Kaplan Meier survival analysis is shown in FIG. 9. Median survival times for animals treated with nanoparticles PEG-mIL12 (21.0 days), PEG-TK-mGMCSF (19.0 days), PEG-TK-hIL15-mIL12 (19.5 days) and PEG-TK-mIL12-flag (19.0 days) were significantly (p≤0.05) longer than 9.5% Trehalose Control (13.5 days). There was no significant difference in median survival for animals treated with PEG-lucia control (15.5 days) and 9.5% Trehalose Control. Therefore, formulations of active nanoparticles at N/P=6 were effective at prolonging survival in LL/2 mice. Accordingly, these results demonstrate that the formulations of the present technology are useful in methods for treating cancer in a subject in need thereof.

### Example 8: Syngeneic Model of Experimental Metastasis to the Lung Using B16F10-Red-FLuc Cells

Tumor cell culture and inoculation—B16F10-Red-FLuc mouse melanoma cells (Perkin Elmer, Waltham, Mass., USA) were cultured in RPMI 1640 cell culture medium supplemented with 10% FBS, 1% GlutaMAX™, and 1% penicillin-streptomycin, and grown at 37° C. in a humidified cell culture incubator supplied with 5% CO₂. The cells were harvested by trypsinization, washed twice in HBSS and counted (using trypan blue exclusion). The final cell density was adjusted with HBSS to 3.5×10e6 cells/mL. 100 μL of cell suspension, consisting of 3.5×10e5 cells, was discharged into the tail vein of mice at the start of the study (Day 0). Imaging was performed on study Day 5, when the presence of lung tumors was confirmed in sufficient animals to commence the study. Imaging was performed as described in Example 6.

9.5% Trehalose buffer and nanoparticles containing PEG-lucia, PEG-mIL12, PEG-TK-mIL12, PEG-mIL2-mIL12 and PEG-TK-mIL2-mIL12 (each 60 μg/vial) were reconstituted in 300 μl, of nuclease-free water per vial on the day of dosing to give dosing solutions of 200 μg/mL. Formulated test articles were stored at 4° C. and used on day of reconstitution.

9.5% Trehalose buffer and nanoparticles containing PEG-lucia, PEG-mIL12, PEG-TK-mIL12, PEG-mIL2-mIL12 and PEG-TK-mIL2-mIL12 were administered via intravenous injection (i.v.) on Study Days 5, 8, 11, 14 and 17. Treatments were administered at a dose of 2 mg/kg in a dosing volume of 10 mL/kg on Study Days 5, 11, 14 and 17. Due to declining body weight in all groups apart from the vehicle control at Day 6, the dose was reduced to 1 mg/kg in 5 mL/kg for the dose administered on Study Day 8. Dosing then resumed at 2 mg/kg in 10 mL/kg on Study Day 11 as per protocol.

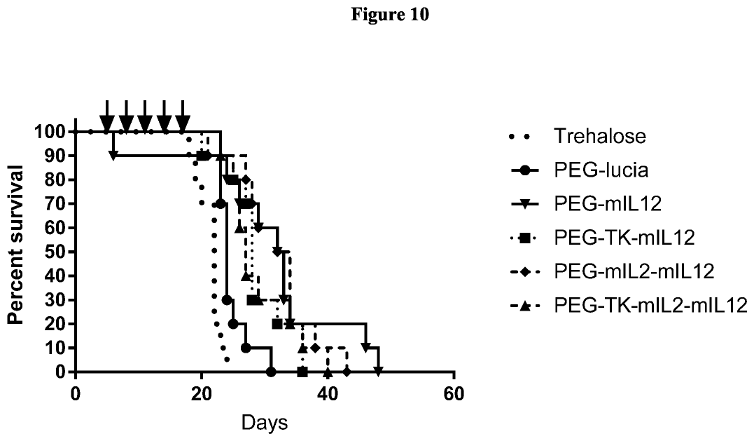

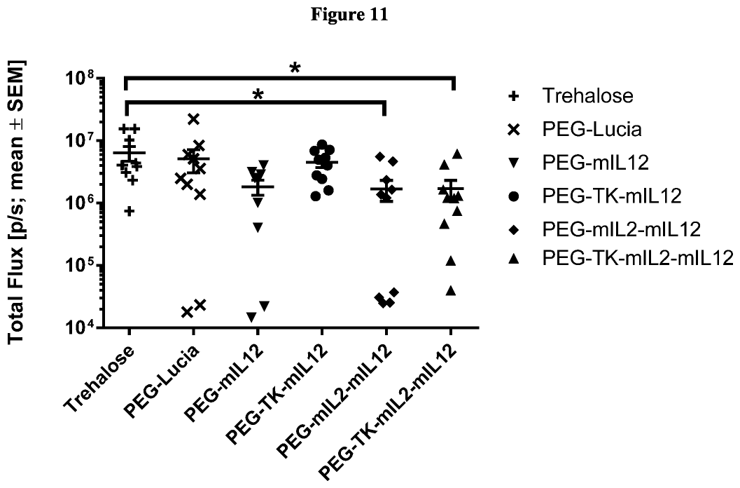

Results—Median survival times for animals treated with PEG-lucia (24.0 days), PEG-mIL12 (32.5 days), PEG-TK-mIL12 (28.0 days), PEG-mIL2-mIL12 (33.0 days), and PEG-TK-mIL2-mIL12 (27.0 days) were significantly (p≤0.05, Log-rank test) longer than 9.5% Trehalose control (22.0 days) (FIG. 10). Survival times were significantly extended (p≤0.05, Log-rank test) for the PEG plasmids expressing IL-12 over PEG-lucia (IL-12 negative control) Luminescence readings on Study Day 12 indicated significant (p≤0.05) inhibition of tumor growth by treatment with PEG-mIL2-mIL12 compared with 9.5% Trehalose control (FIG. 11) and a trend towards significance for PEG-mIL12 (p=0.054). Accordingly, these results demonstrate that the formulations of the present technology are useful in methods for treating cancer in a subject in need thereof.

### Example 9: Syngeneic Model of Experimental Metastasis to the Lung Using B16F10-Red-Fluc Cells

The anti-tumor effect of nanoparticles containing PEG-mIL12 and expressing mIL-12 was compared to recombinant mIL-12 protein administered subcutaneously. The experimental design was as Example 8 but nanoparticles were prepared at N/P=4 and N/P=6 ratios. Nanoparticles were dosed as before for N/P=6, however, for the N/P=4 formulation the dose was maintained at 2 mg/kg in 10 mL/kg at day 8. For dosing of the recombinant protein, 10 μg of recombinant mIL-12 (PeproTech, Rocky Hill, N.J., USA) were reconstituted in PBS to make a 100 μg/mL stock solution. Dosing of the animals was at 4 μg/kg for the initial dose (Day 5) followed by four subsequent doses 12 μg/kg at the same intervals as the nanoparticles (Day 8, 11, 14 and 17).

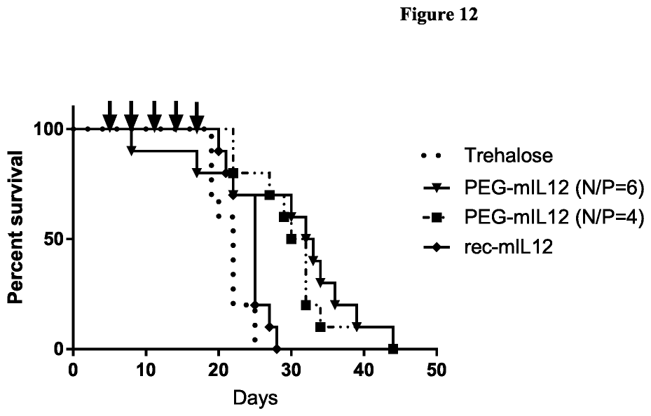

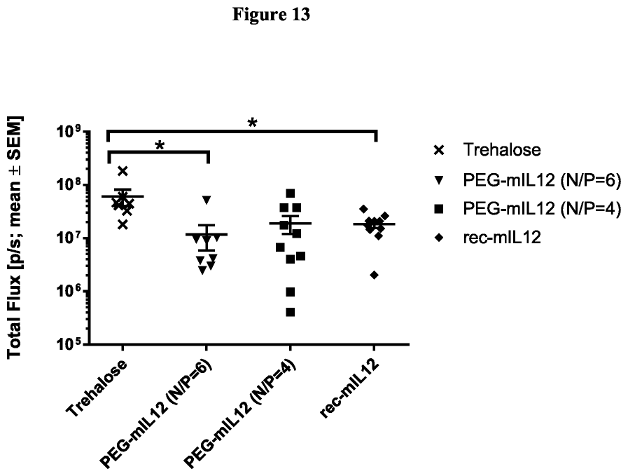

Results—Median survival times for animals treated with PEG-mIL12 nanoparticles (N/P=6) (32.5 days), PEG-mIL12 nanoparticles (N/P=4) (31.0 days) and recombinant mIL-12 (25.0 days) were significantly longer (p≤0.01 for formulations of PEG-mIL12 and p≤0.05 for recombinant mIL-12, Log-rank test) than the Trehalose control (22.0 days). Additionally, the median survival time for the PEG-mIL12 nanoparticles was significantly (p≤0.05, Log-rank test) longer than recombinant mIL-12 at the dose tested (FIG. 12). The dose of recombinant IL-12 protein was chosen so that the toxic side-effects of recombinant IL-12 would be minimized in this study, yet the protein would still be therapeutically effective (Yue et al., 2016, BMC Cancer 16:665; Car et al., 1999, Tox. Pathology 27, 58-63). Luminescence readings on Study Day 19 indicated significant (p≤0.05) inhibition of tumor growth by treatment with PEG-mIL2 (N/P=6) and rec-mIL12 compared with 9.5% Trehalose control and a trend towards significance for PEG-mIL12 (N/P=4) (p=0.0538) (FIG. 13). Accordingly, these results demonstrate that the formulations of the present technology are useful in methods for treating cancer in a subject in need thereof.

### Example 10: Evaluation of Anti-PD1 Antibody in an Experimental Metastasis to the Lung Using B16F10-Red-Fluc Cells

The murine anti-PD1, iTME-0006-0002 (WO2016/170039), sequence was reverse-translated into a CpG-free DNA sequence and synthesized in fusion with mIL-12 or alone with 5′ HindIII site and a 3′ stop codon and a NheI site (SEQ ID NO: 19 and SEQ ID NO: 23, respectively). The cassette, iTME, is cloned into a pCpGfree plasmid (Invivogen, Carlsbad, Calif., USA) containing the PEG-3 promoter to create PEG-iTME and PEG-mIL12-iTME and formulated into nanoparticles with PEI as described in Example 9. The nanoparticles are administered intravenously as previously described in mice harboring experimental metastases to the lung with B16F10-Red-Fluc cells. The effect of PEG-iTME and PEG-mIL12-iTME nanoparticles on survival and tumor growth is compared against Trehalose vehicle control and anti-murine PD-1, RMP1-14 (#14-9982-81, Thermofisher Waltham, Mass., USA) monoclonal antibody intravenously dosed at 4 mg/kg at each dosing point. It is predicted that the nanoparticles PEG-iTME and PEG-mIL12-iTME prolong survival of mice harboring metastatic tumors in the lung and are as effective or more effective than RMP1-14 monoclonal antibody. The same effect is anticipated in man when using recombinant humanized monoclonal antibodies alone or with human IL-12.

### Example 11: In Vivo Bioluminescence Imaging in the NSG-LL2 and NSG-B16F10 Models with CpG Containing and CpG-Free Payload

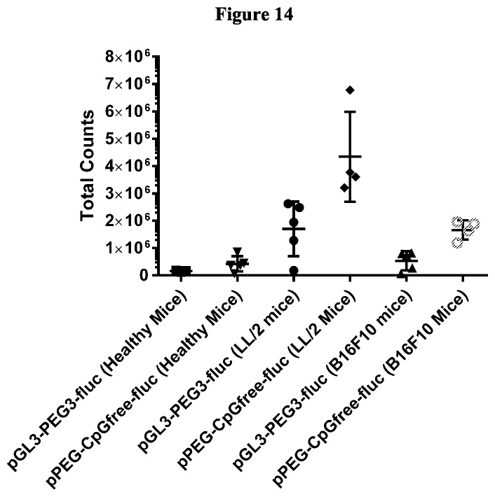

Either LL/2 or B16F10 cells were injected via the tail vein into 6-8 week old NSG mice (10e6 cells per mouse) and were left to infect in the lungs for approximately one week for LL/2 and two weeks for B16F10. Two plasmids were used to determine tumor specific expression in the context of CpG burden of PEG-3 containing plasmids: one plasmid, pGL3-PEG3-fluc, contains 357 CpG sites within the plasmid backbone and the luciferase gene whose expression is driven by the PEG-3 promoter, and the second plasmid pPEG-CpGfree-fluc, is CpG free except for 43 CpG-sequences within the PEG3 promoter. The plasmids were formulated with in vivo-jetPE10 (N/P=6) and the nanoparticles were injected into non-tumor bearing NSG mice or mice containing NSG-LL/2 and NSG-B16F10 tumors (40 μg of plasmid per mouse). BLI imaging was performed 48 h post-injection of the nanoparticles as follows: the mice were injected (i.p.) with 100 μL of D-luciferin (25 mg/mL in sterile PBS) and anesthetized with isoflurane (3%). Six minutes after the injection of D-luciferin, the mice were imaged for a duration of 3 min using the IVIS Spectrum Imaging System (Perkin Elmer) for bioluminescence signals. The region of interest was drawn to cover the entire lung region of each mouse and total flux (photon counts/sec) was calculated to determine the expression of the fLuc (FIG. 14). In the LL/2 and B16F10 models, the pPEG-CpGfree-fluc group has significantly (p≤0.05, unpaired T-test) more counts, corresponding to greater expression of firefly luciferase than in animals treated with the pGL3-PEG3-fluc plasmid. There was no significant difference between the luciferase expression of the two plasmid formulations in healthy animals indicating there was no difference in background expression.

### Example 12: In Vivo Toxicity of CpGhigh Versus CpGlow Plasmids

To further evaluate the benefit of reducing CpG within the plasmid and payload, an experiment was conducted to determine if there was a significant difference between a plasmid containing 43 CpG sites from the PEG-3 promoter (pCpGfree-PEG-TK) and an alternative plasmid containing 357 CpG sites pGL3-PEG3-fluc (a CpGhigh plasmid). Both plasmids were formulated with in vivo jetPEI® (Polyplus) N/P=6 and were injected into CD1 mice via the tail vein. Inflammatory response was determined by assay of the acute inflammatory cytokines IL-12, TNF-α, and IFN-γ. Although there was a cytokine response from both nanoparticle formulations, the pCpGfree-PEG-TK plasmid (CpGlow) showed a significant reduction in the induction of endogenous IL-12, TNF-α, and IFN-γ in serum compared with those resulting from CpG-containing pCpGfree-PEG-TK (Table 4). In particular, endogenous IL-12 induction was at least 100-fold less and IFN-γ at least 3-fold less, on average, for the CpGlow plasmid compared to the CpGhigh plasmid, therefore demonstrating greater safety for the CpGlow plasmid formulation.

TABLE 4

| Endogenous | | | | | | | | |
|---|---|---|---|---|---|---|---|---|
| murine | Nanoparticle | | | | | | | |
| cytokine | formulation | Mouse1 | Mouse2 | Mouse3 | Mouse4 | Mouse5 | Average | SD |
| IL-12 | Non-injected | ND | ND | ND | ND | ND | | |
| (sensitivity: | pGL3-PEG3-fluc | 1202.9 | 593.9 | 747 | 989.4 | 972.4 | 901.1 | 235.6 |
| 7.8 pg/mL) | pCpGfree-PEG-TK | BLQ | ND | ND | ND | BLQ | <7.8 | |
| TNF-α | Non-injected | ND | ND | ND | ND | ND | | |
| (sensitivity: | pGL3-PEG3-fluc | 30.3 | 39.9 | 41.8 | 53.2 | 49.4 | 42.9 | 8.9 |
| 10.9 pg/mL) | pCpGfree-PEG-TK | 21.2 | 30.2 | 26.7 | 38.8 | 27.3 | 28.8 | 6.5 |
| IFN-γ | Non-injected | ND | ND | ND | ND | ND | | |
| (sensitivity: | pGL3-PEG3-fluc | 23108.9 | 17320 | 15293.3 | 22882.2 | 20404.4 | 19801.8 | 3437.8 |
| 9.3 pg/mL) | pCpGfree-PEG-TK | 6091.1 | 8520 | 5848.9 | 5633.3 | 5293.3 | 6277.3 | 1287.5 |

ND: Not detected.
BLQ: Below the limit of quantification.

### Example 13: Activity of PEG-hIL12 and PEG-hIL24 in Humanized (CD34+) NSG Mice

Twelve animals (CD34⁺ HU-NSG™ mice) humanized from CD34⁺ cells from a single human umbilical cord donor (Jackson Laboratory, Bar Harbor, Me., US) were inoculated while under isoflurane inhalation anesthesia (Study Day 0) with 10e6 MDA-MB-231-luc2 cells (Perkin Elmer, Waltham, Mass., US) via the tail vein. Animals were randomized using a matched pair distribution method based on body weight prior to administration of the test articles on day 4. Imaging for in vivo luminescence signal in the thoracic region on Day 8 confirmed the presence of lung tumours. The nanoparticles that were tested were formulated with PEG-lucia, PEG-hIL12 and PEG-IL24 and in vivo-jetPEI. Nanoparticles were administered at 1.5 mg/mL following reconstitution in ultrapure nuclease free water in a dosing volume of 7.5 mL/kg. on study days 4, 7, 10, 13, 16, and 19.

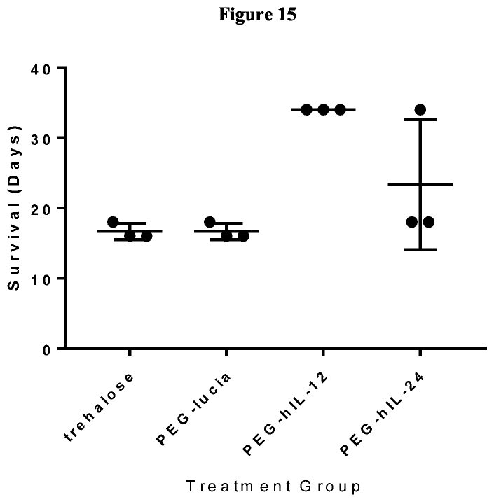

Animals were assessed daily for clinical condition and body weight loss in accordance with ethical guidelines: body weight loss exceeding 15% of initial body weight, or the presence of severe adverse clinical and/or physical signs of toxicity in any animal were considered as criteria for cessation of treatment to the entire group. The animals were monitored over a period of 32 days and survival was noted. As observed from the survival data, individual animals treated with nanoparticles harbouring the PEG-hIL12 and PEG-hIL24 plasmids survived longer compared to animals in the control groups (FIG. 15). Accordingly, these results demonstrate that the formulations of the present technology are useful in methods for treating cancer in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

SEQUENCE LISTING
For the sequences provided herein: [symbol]
= restriction endonuclease cleavage sites used
for cloning.
HSV1-TK CpGfree (NcoI-BamHI-NheI)
>SEQ ID NO: 1 [symbol]
CTTCTTACCCTGGACACCAGCATGCTTCTGCCTTTGACCAGGCTGCCAG
ATCCAGGGGCCACTCCAACAGGAGAACTGCCCTAAGACCCAGAAGACAGCAGG
AAGCCACTGAGGTGAGGCCTGAGCAGAAGATGCCAACCCTGCTGAGGGTGTACA
TTGATGGACCTCATGGCATGGGCAAGACCACCACCACTCAACTGCTGGTGGCACT
GGGCTCCAGGGATGACATTGTGTATGTGCCTGAGCCAATGACCTACTGGAGAGT
GCTAGGAGCCTCTGAGACCATTGCCAACATCTACACCACCCAGCACAGGCTGGA
CCAGGGAGAAATCTCTGCTGGAGATGCTGCTGTGGTGATGACCTCTGCCCAGATC
ACAATGGGAATGCCCTATGCTGTGACTGATGCTGTTCTGGCTCCTCACATTGGAG
GAGAGGCTGGCTCTTCTCATGCCCCTCCACCTGCCCTGACCCTGATCTTTGACAG
ACACCCCATTGCAGCCCTGCTGTGCTACCCAGCAGCAAGGTACCTCATGGGCTCC
ATGACCCCACAGGCTGTGCTGGCTTTTGTGGCCCTGATCCCTCCAACCCTCCCTG
GCACCAACATTGTTCTGGGAGCACTGCCTGAAGACAGACACATTGACAGGCTGG
CAAAGAGGCAGAGACCTGGAGAGAGACTGGACCTGGCCATGCTGGCTGCAATCA
GAAGGGTGTATGGACTGCTGGCAAACACTGTGAGATACCTCCAGTGTGGAGGCT
CTTGGAGAGAGGACTGGGGACAGCTCTCTGGAACAGCAGTGCCCCCTCAAGGAG
CTGAGCCCCAGTCCAATGCTGGTCCAAGACCCCACATTGGGGACACCCTGTTCAC
CCTGTTCAGAGCCCCTGAGCTGCTGGCTCCCAATGGAGACCTGTACAATGTGTTT
GCCTGGGCTCTGGATGTTCTAGCCAAGAGGCTGAGGTCCATGCATGTGTTCATCC
TGGACTATGACCAGTCCCCTGCTGGATGCAGAGATGCTCTGCTGCAACTAACCTC
TGGCATGGTGCAGACCCATGTGACCACCCCTGGCAGCATCCCCACCATCTGTGAC
CTAGCCAGAACCTTTGCCAGGGAGATGGGAGAGGCCAAT [symbol] TGATAA

[symbol]

[symbol]

SR39 (NcoI-BamHI-NheI)
>SEQ ID NO: 2 [symbol]
CTTCTTACCCTGGACACCAGCATGCTTCTGCCTTTGACCAGGCTGCCAG
ATCCAGGGGCCACTCCAACAGGAGAACTGCCCTAAGACCCAGAAGACAGCAGG
AAGCCACTGAGGTGAGGCCTGAGCAGAAGATGCCAACCCTGCTGAGGGTGTACA
TTGATGGACCTCATGGCATGGGCAAGACCACCACCACTCAACTGCTGGTGGCACT
GGGCTCCAGGGATGACATTGTGTATGTGCCTGAGCCAATGACCTACTGGAGAGT
GCTAGGAGCCTCTGAGACCATTGCCAACATCTACACCACCCAGCACAGGCTGGA
CCAGGGAGAAATCTCTGCTGGAGATGCTGCTGTGGTGATGACCTCTGCCCAGATC
ACAATGGGAATGCCCTATGCTGTGACTGATGCTGTTCTGGCTCCTCACATTGGAG
GAGAGGCTGGCTCTTCTCATGCCCCTCCACCTGCCCTGACCATTTTCCTGGACAG
ACATCCCATTGCCTTCATGCTGTGCTACCCAGCAGCAAGGTACCTCATGGGCTCC
ATGACCCCACAGGCTGTGCTGGCTTTTGTGGCCCTGATCCCTCCAACCCTCCCTG
GCACCAACATTGTTCTGGGAGCACTGCCTGAAGACAGACACATTGACAGGCTGG
CAAAGAGGCAGAGACCTGGAGAGAGACTGGACCTGGCCATGCTGGCTGCAATCA
GAAGGGTGTATGGACTGCTGGCAAACACTGTGAGATACCTCCAGTGTGGAGGCT
CTTGGAGAGAGGACTGGGGACAGCTCTCTGGAACAGCAGTGCCCCCTCAAGGAG
CTGAGCCCCAGTCCAATGCTGGTCCAAGACCCCACATTGGGGACACCCTGTTCAC
CCTGTTCAGAGCCCCTGAGCTGCTGGCTCCCAATGGAGACCTGTACAATGTGTTT
GCCTGGGCTCTGGATGTTCTAGCCAAGAGGCTGAGGTCCATGCATGTGTTCATCC
TGGACTATGACCAGTCCCCTGCTGGATGCAGAGATGCTCTGCTGCAACTAACCTC
TGGCATGGTGCAGACCCATGTGACCACCCCTGGCAGCATCCCCACCATCTGTGAC
CTAGCCAGAACCTTTGCCAGGGAGATGGGAGAGGCCAAT [symbol] TGATAA

[symbol]

[symbol]

human IL-2 (HindIII-NheI)
>SEQ ID NO: 3 [symbol]
GGCATTCCGGTACTGTTGGTAAAGCCACCATGTACAGGATGCAACTCCT
GTCTTGCATTGCACTGAGTCTTGCACTTGTCACAAACAGTGCACCTACTTCAAGT
TCTACAAAGAAAACACAGCTGCAACTGGAGCATCTCCTGCTGGATCTGCAGATG
ATCTTGAATGGAATTAATAATTACAAGAATCCCAAACTCACCAGGATGCTCACAT
TTAAGTTTTACATGCCCAAGAAGGCCACAGAACTGAAACATCTTCAGTGTCTGGA
AGAAGAACTCAAACCTCTGGAGGAAGTGCTCAATCTGGCTCAAAGCAAAAACTT
TCACCTGAGACCCAGGGACCTGATCAGCAATATCAATGTAATTGTTCTGGAACTC
AAGGGATCTGAAACAACATTCATGTGTGAATATGCTGATGAGACAGCAACCATT
GTGGAATTTCTGAACAGATGGATTACCTTTTGTCAAAGCATCATCTCAACACTGACTTAA
ACCTGA [symbol]
murine IL-2 (HindIII-NheI)
>SEQ ID NO: 4 [symbol]
GGCATTCCGGTACTGTTGGTAAAGCCACCATGTACAGCATGCAGCTGGC
CTCCTGTGTGACACTGACACTGGTGCTGCTGGTGAACTCTGCACCCACTTCAAGC
TCCACCTCAAGCTCTACAGCTGAAGCCCAGCAGCAGCAGCAGCAGCAGCAGCAG
CAGCAGCAGCACCTGGAGCAGCTGCTGATGGACCTGCAGGAGCTGCTGAGCAGG
ATGGAGAATTACAGGAACCTGAAACTCCCCAGGATGCTGACCTTCAAATTTTACT
TGCCCAAGCAGGCCACAGAACTGAAGGATCTGCAGTGCCTGGAAGATGAACTTG
GACCTCTGAGGCATGTGCTGGATCTGACTCAAAGCAAGAGCTTTCAACTGGAAG
ATGCTGAGAATTTCATCAGCAATATCAGAGTGACTGTGGTCAAACTGAAGGGCT
CTGACAACACATTTGAGTGCCAATTTGATGATGAGTCAGCCACTGTGGTGGACTT
TCTGAGGAGATGGATTGCCTTCTGTCAAAGCATCATCTCAACAAGCCCTCAATAAACCTG
A [symbol]
human scIL-12 (NotI-NheI)
>SEQ ID NO: 5 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCTAAACCTGA

[symbol]

murine scIL-12 (HindIII-NheI)
>SEQ ID NO: 6 [symbol]
GGCATTCCGGTACTGTTGGTAAAGCCACCATGTGTCCTCAGAAGCTCAC
CATCTCCTGGTTTGCCATTGTTTTGCTGGTGTCTCCACTCATGGCCATGTGGGAGC
TGGAGAAAGATGTTTATGTTGTGGAGGTGGACTGGACTCCTGATGCCCCTGGAGA
AACAGTGAACCTCACCTGTGACACCCCTGAAGAAGATGACATCACCTGGACCTC
AGACCAGAGACATGGAGTCATAGGCTCTGGAAAGACCCTGACCATCACTGTCAA
AGAGTTTCTGGATGCTGGCCAGTACACCTGCCACAAAGGAGGGGAGACTCTGAG
CCACTCACATCTGCTGCTCCACAAGAAGGAAAATGGAATTTGGTCCACTGAAATT
CTGAAAAATTTCAAAAACAAGACTTTCCTGAAGTGTGAAGCACCAAATTACTCTG
GAAGGTTCACCTGCTCATGGCTGGTGCAAAGAAACATGGACTTGAAGTTCAACA
TCAAGAGCAGTAGCAGTTCCCCTGACTCTAGGGCAGTGACATGTGGAATGGCCT
CTCTGTCTGCAGAGAAGGTCACACTGGACCAAAGGGACTATGAGAAGTATTCAG
TGTCCTGCCAGGAGGATGTCACCTGCCCAACTGCTGAGGAGACCCTGCCCATTGA
ACTGGCCTTGGAAGCAAGGCAGCAGAATAAATATGAGAACTACAGCACCAGCTT
CTTCATCAGGGACATCATCAAACCAGACCCTCCCAAGAACTTGCAGATGAAGCC
TTTGAAGAACTCACAGGTGGAGGTCAGCTGGGAGTACCCTGACTCCTGGAGCAC
TCCCCATTCCTACTTCTCCCTCAAGTTCTTTGTTAGAATCCAGAGGAAGAAAGAA
AAGATGAAGGAGACAGAGGAGGGGTGTAACCAGAAAGGTGCCTTCCTGGTGGA
GAAGACATCTACAGAAGTCCAATGCAAAGGAGGGAATGTCTGTGTGCAAGCTCA
GGATAGGTATTACAATTCCTCATGCAGCAAGTGGGCATGTGTTCCCTGCAGGGTC
AGATCTGGTGGAGGTGGAAGTGGAGGTGGTGGATCTGGGGGTGGAGGCAGCAG
GGTCATTCCAGTCTCTGGACCTGCCAGGTGTCTTAGCCAGTCCAGAAACCTGCTG
AAGACCACAGATGACATGGTGAAGACTGCCAGAGAAAAACTGAAACATTATTCC
TGCACTGCTGAAGACATTGATCATGAAGACATCACAAGGGACCAAACCAGCACA
TTGAAGACCTGTCTGCCACTGGAACTGCACAAGAATGAGAGTTGCCTGGCTACTA
GAGAGACTTCTTCCACAACAAGAGGGAGCTGCCTGCCCCCACAGAAGACCTCTT
TGATGATGACCCTGTGCCTTGGTAGCATCTATGAGGACTTGAAGATGTACCAGAC
AGAGTTCCAGGCCATCAATGCAGCACTTCAGAATCACAACCATCAGCAGATCAT
TCTGGACAAGGGCATGCTGGTGGCCATTGATGAGCTGATGCAGTCTCTGAATCAT
AATGGAGAGACTCTGAGACAGAAACCTCCTGTGGGAGAAGCAGACCCTTACAGA
GTGAAAATGAAGCTCTGCATCCTGCTTCATGCCTTCAGCACCAGAGTGGTGACCATCAAC
AGGGTGATGGGCTATCTGAGCTCTGCCTAAACCTGA [symbol]
human IL-15 (Esp3I-NheI)
>SEQ ID NO: 7 [symbol]
TATGTACAGGATGCAACTCCTGTCTTGCATTGCACTGAGTCTTGC
ACTTGTCACAAACAGTGCAGGAGCCAACTGGGTGAATGTGATCAGTGATTTGAA
AAAAATTGAAGATCTTATTCAATCTATGCATATTGATGCTACTTTGTATACAGAA
AGTGATGTTCACCCCAGTTGCAAAGTGACAGCAATGAAGTGCTTTCTCTTGGAGC
TGCAAGTTATTTCACTTGAGTCTGGAGATGCAAGTATTCATGATACAGTGGAAAA
TCTGATCATCCTGGCAAACAACAGTTTGTCTTCTAATGGGAATGTGACAGAATCT
GGATGCAAAGAATGTGAGGAACTGGAGGAAAAAAATATTAAAGAATTTTTGCAG
AGTTTTGTGCATATTGTCCAAATGTTCATCAACACTTCTTAAACCTGA [symbol]
human IL-24 (HindIII-NheI)
>SEQ ID NO: 8 [symbol]
GGCATTCCGGTACTGTTGGTAAAGCCACCATGAATTTTCAACAGAGGCT
GCAAAGCCTGTGGACTCTGGCCAGACCCTTCTGCCCTCCTTTGCTGGCCACAGCC
TCTCAAATGCAGATGGTTGTGCTCCCTTGCCTGGGTTTTACCCTGCTTCTCTGGAG
CCAGGTGTCAGGGGCCCAGGGCCAAGAATTCCACTTTGGGCCCTGCCAAGTGAA
GGGGGTTGTTCCCCAGAAACTGTGGGAAGCCTTCTGGGCTGTGAAAGACACTAT
GCAAGCTCAGGATAACATCACCAGTGCCAGGCTGCTGCAGCAGGAGGTTCTGCA
GAATGTCTCTGATGCTGAGAGCTGTTACCTTGTCCACACCCTGCTGGAGTTCTAC
TTGAAAACTGTTTTCAAAAACTACCACAATAGAACAGTTGAAGTCAGGACTCTG
AAGTCATTCTCTACTCTGGCCAACAACTTTGTTCTCATTGTGTCACAACTGCAACC
CAGTCAAGAAAATGAGATGTTTTCCATCAGAGACAGTGCACACAGGAGGTTTCT
GCTGTTCAGAAGAGCATTCAAACAGTTGGATGTGGAAGCAGCTCTGACCAAAGC
CCTTGGGGAAGTGGACATTCTTCTGACCTGGATGCAGAAATTCTACAAGCTCTAAACCTG
A [symbol]
P2A-human GM-CSF (BamHI-NheI)
>SEQ ID NO: 9 [symbol]
TCTGGAAGTGGAGCTACTAACTTCAGCCTGCTGAAGCAGGCTGGAGAT
GTGGCTGCAGAGCCTGCTGCTCTTGGGCACTGTGGCCTGCAGCATCTCTGCACCT
GCCAGAAGCCCCAGCCCCAGCACCCAGCCCTGGGAGCATGTGAATGCCATCCAG
GAGGCCAGGAGGCTCCTGAACCTGAGTAGAGACACTGCTGCTGAGATGAATGAA
ACAGTGGAAGTCATCTCAGAAATGTTTGACCTCCAGGAGCCCACCTGCCTCCAGA
CCAGGCTGGAGCTGTACAAGCAGGGCCTGAGGGGCAGCCTCACCAAGCTCAAGG
GCCCCTTGACCATGATGGCCAGCCACTACAAGCAGCACTGCCCTCCAACCCCTGA
AACTTCCTGTGCAACCCAGATTATCACCTTTGAAAGTTTCAAAGAGAACCTGAAG
GACTTTCTGCTTGTCATCCCCTTTGACTGCTGGGAGCCAGTCCAGGAGTGAACCTGA

[symbol]

P2A-murine GM-CSF (BamHI-NheI)
>SEQ ID NO: 10 [symbol]
TCTGGAAGTGGAGCTACTAACTTCAGCCTGCTGAAGCAGGCTGGAGAT
GTGGAGGAGAACCCTGGACCTATGTGGCTGCAGAATCTGCTTTTCCTGGGCATTG
TGGTCTACAGCCTCTCAGCACCCACCAGGTCACCCATCACTGTCACCAGACCTTG
GAAGCATGTAGAGGCCATCAAAGAAGCCCTGAACCTCCTGGATGACATGCCTGT
CACCTTGAATGAAGAGGTAGAAGTGGTCTCTAATGAGTTCTCCTTCAAGAAGCTG
ACATGTGTGCAGACCAGACTGAAGATATTTGAGCAGGGTCTAAGGGGCAATTTC
ACCAAACTCAAGGGAGCCTTGAACATGACAGCCAGCTACTACCAGACATACTGC
CCCCCAACTCCTGAAACAGACTGTGAAACACAAGTTACCACCTATGCTGATTTCA
TAGACAGCCTTAAAACCTTTCTGACTGATATCCCCTTTGAATGCAAAAAACCAGGCCAAA
AATGAACCTGA [symbol]
P2A-PD1ECD-FcFurin (BamII-Esp3I)
>SEQ ID NO: 11 [symbol]
TCTGGAAGTGGAGCTACTAACTTCAGCCTGCTGAAGCAGGCTGGAGAT
GTGGAGGAGAACCCTGGACCTATGGACTGGACCTGGAGGGTCTTCTGTTTGCTGG
CTGTAACTCCAGGTGCCCACCCCCTGGACTCCCCAGACAGGCCCTGGAACCCCCC
CACCTTCTCCCCAGCCCTGCTGGTGGTGACTGAAGGGGACAATGCCACCTTCACC
TGCAGCTTCTCCAACACATCTGAGAGCTTTGTGCTGAACTGGTACAGGATGAGCC
CCAGCAACCAGACTGACAAGCTGGCTGCCTTCCCTGAGGACAGGAGCCAGCCTG
GCCAGGACTGCAGATTCAGGGTCACACAACTGCCCAATGGGAGGGACTTCCACA
TGAGTGTGGTCAGGGCCAGGAGAAATGACAGTGGCACCTACCTCTGTGGGGCCA
TCTCCCTGGCCCCCAAGGCCCAGATCAAAGAGAGCCTGAGGGCAGAGCTCAGGG
TGACAGAGAGAAGGGCAGAAGTGCCCACAGCCCACCCCAGCCCCTCACCCAGGT
CAGCTGGCCAGTTCCAAACCCTGGTGGAGTCCAAATATGGTCCCCCATGCCCACC
ATGCCCAGCACCTGAGTTTGAGGGGGGACCATCAGTCTTCCTGTTCCCCCCAAAA
CCCAAGGACACTCTCATGATCTCCAGGACCCCTGAGGTCACCTGTGTGGTGGTGG
ATGTGAGCCAGGAAGACCCTGAGGTCCAGTTCAACTGGTATGTGGATGGGGTGG
AGGTGCATAATGCCAAGACAAAGCCTAGGGAGGAGCAGTTCAACAGCACCTACA
GAGTGGTCAGTGTCCTCACAGTCCTGCACCAGGACTGGCTGAATGGCAAGGAGT
ACAAGTGCAAGGTCTCCAACAAAGGCCTCCCCTCCTCCATTGAGAAAACCATCTC
CAAAGCCAAAGGGCAGCCCAGAGAGCCACAGGTGTACACCCTGCCCCCATCCCA
GGAGGAGATGACCAAGAACCAGGTCAGCCTGACCTGCCTGGTCAAAGGCTTCTA
CCCCAGTGACATTGCTGTGGAGTGGGAGAGCAATGGGCAGCCTGAGAACAACTA
CAAGACCACACCTCCAGTGCTGGACTCTGATGGCTCCTTCTTCCTCTACAGCAGG
CTCACAGTGGACAAGAGCAGGTGGCAGGAGGGGAATGTCTTCTCATGCTCTGTG
ATGCATGAGGCTCTGCACAACCACTACACACAGAAGAGCCTCTCCCTGTCTCTGG
GTAAAAGAAGGAAGAGGGGAAGTGGAGAGGGCAGAGGAAGTCTGCTAACATGTGGTGATG
TGGAGGAGAATCCTG [symbol]
FliC Esp3I-NheI
>SEQ ID NO: 12 [symbol]
TATGGAGACAGACACACTCCTGCTGTGGGTGCTGCTGCTCTGGG
TTCCAGGTTCCACTGGTGACATGGCACAAGTCATTAATACAAACAGCCTGTCTCT
GTTGACCCAGAATAACCTGAACAAATCCCAGTCAGCTCTGGGCACAGCTATTGA
GAGACTGTCTTCTGGTCTGAGGATCAACAGTGCCAAAGATGATGCTGCAGGTCA
GGCCATTGCTAACAGGTTTACTGCCAACATCAAAGGTCTGACTCAGGCTTCCAGA
AATGCTAATGATGGTATCTCCATTGCCCAGACCACTGAAGGAGCTCTGAATGAA
ATCAACAACAACCTGCAGAGAGTGAGGGAACTGGCTGTTCAGTCTGCTAACAGC
ACCAACTCCCAGTCTGACCTGGACTCCATCCAGGCTGAAATCACCCAGAGACTG
AATGAAATTGACAGAGTGTCTGGCCAGACTCAGTTCAATGGAGTGAAAGTCCTG
GCCCAGGACAACACCCTGACCATCCAGGTTGGTGCCAATGATGGTGAAACTATT
GATATTGATCTGAAGCAGATCAACTCTCAGACCCTGGGTCTGGATACCCTGAATG
TGCAACAAAAATATAAGGTCAGTGATACAGCTGCAACTGTTACAGGATATACTC
AAAATAAAGATGGTTCCATCAGTATTAATACTACAAAATACACTGCAGATGATG
GTACATCCAAAACTGCACTGAACAAACTGGGTGGGGCAGATGGCAAAACAGAA
GTTGTTTCTATTGGTGGTAAAACTTATGCTGCAAGTAAAGCTGAAGGTCACAACT
TTAAAGCACAGCCTGATCTGGCTGAAGCTGCTGCTACAACCACAGAAAACCCTCT
GCAGAAAATTGATGCTGCTTTGGCACAGGTTGACACCCTGAGATCTGACCTGGGT
GCTGTGCAGAACAGGTTCAACTCTGCTATTACCAACCTGGGCAACACAGTGAAC
AACCTGACTTCTGCCAGAAGCAGGATTGAAGATTCTGACTATGCCACAGAAGTTT
CCAACATGTCTAGAGCCCAGATTCTGCAGCAGGCTGGTACCTCTGTTCTGGCCCA
GGCCAACCAGGTTCCCCAAAATGTCCTCTCTCTGCTGAGATAAACCTGA [symbol]
hIL-12-Ipilimumab (NotI-NheI)
>SEQ ID NO: 13 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCAGGAGAAA
GAGAGGATCCTCTGGAAGTGGAGCTACTAACTTCAGCCTGCTGAAGCAGGCTGG
AGATGTGGAGGAGAACCCTGGACCTATGGAGTTTGGGCTGAGCTGGGTTTTCCTT
GTTGCTCTTTTAAGAGGTGTCCAGTGTCAGGTGCAGTTGGTGGAGTCTGGGGGGG
GTGTGGTGCAGCCAGGGAGGTCACTGAGACTGAGTTGTGCAGCAAGTGGGTTTA
CATTTAGTAGTTATACAATGCATTGGGTTAGGCAAGCTCCAGGGAAGGGTCTGGA
GTGGGTGACTTTTATTTCTTATGATGGTAATAATAAATATTATGCAGATTCAGTTA
AGGGAAGGTTTACTATTAGTAGGGATAATTCAAAAAATACTCTGTATTTGCAGAT
GAATTCTCTGAGGGCTGAGGATACAGCTATTTATTATTGTGCTAGAACTGGTTGG
CTGGGTCCATTTGATTATTGGGGGCAGGGAACACTTGTGACAGTGTCATCAGCTT
CAACAAAAGGTCCATCTGTTTTTCCATTGGCTCCTTCTTCTAAGTCAACTTCTGGT
GGAACTGCAGCTCTGGGATGTCTGGTGAAGGATTATTTTCCAGAACCTGTGACTG
TTTCTTGGAATAGTGGTGCTCTGACTAGTGGAGTTCATACTTTTCCAGCTGTTCTG
CAGAGTTCTGGACTGTATTCTCTGAGTAGTGTGGTTACAGTTCCATCAAGTTCTCT
GGGTACTCAAACTTATATTTGTAATGTGAATCATAAGCCTTCAAATACAAAGGTG
GATAAAAGGGTGGAGCCAAAGTCATGTGATAAGACTCATACATGTCCTCCATGT
CCTGCTCCAGAGCTTCTGGGGGGGCCATCTGTTTTTCTGTTTCCACCAAAGCCTA
AGGATACTCTTATGATTAGTAGGACACCAGAAGTTACATGTGTGGTGGTTGATGT
GTCTCATGAAGATCCAGAGGTGAAGTTTAATTGGTATGTTGATGGGGTGGAGGTT
CATAATGCAAAGACAAAGCCTAGGGAGGAACAGTATAATAGTACATATAGAGTG
GTGTCTGTGCTGACTGTGCTGCATCAGGATTGGCTGAATGGAAAAGAGTATAAGT
GTAAAGTGTCAAATAAGGCTCTGCCTGCACCTATTGAAAAAACAATTTCAAAGG
CAAAAGGGCAGCCAAGGGAGCCTCAAGTTTATACTCTGCCACCTTCAAGGGATG
AACTTACAAAGAATCAAGTGAGTTTGACTTGTCTTGTGAAAGGATTTTATCCTTC
AGATATTGCTGTGGAGTGGGAGTCAAATGGTCAGCCTGAAAATAATTATAAGAC
TACTCCACCAGTGCTGGATAGTGATGGGTCTTTTTTTCTGTATAGTAAGCTGACTG
TGGATAAGTCTAGGTGGCAGCAGGGAAATGTGTTTTCTTGTAGTGTGATGCATGA
GGCTCTGCATAATCATTATACACAGAAGTCTCTGAGTTTGTCTCCTGGTAAAAGA
AGGAAGAGGGGAAGTGGAGAGGGCAGAGGAAGTCTGCTAACATGTGGTGATGT
GGAGGAGAATCCTGGACCTATGGACATGAGGGTCCCTGCTCAGCTCCTGGGGCT
CCTGCTGCTCTGGCTCTCAGGTGCCAGATGTGAGATTGTGCTGACACAATCTCCA
GGAACTTTGAGTCTGTCTCCAGGTGAGAGGGCTACACTGTCATGTAGGGCATCAC
AGTCTGTTGGAAGTTCTTATCTGGCTTGGTATCAACAAAAGCCTGGGCAGGCTCC
AAGACTGCTGATTTATGGTGCTTTTTCTAGAGCTACTGGAATTCCTGATAGGTTTA
GTGGGAGTGGGAGTGGAACAGATTTTACACTGACTATTTCTAGACTGGAACCAG
AAGATTTTGCAGTGTATTATTGTCAGCAGTATGGGTCTTCACCTTGGACTTTTGGT
CAGGGAACTAAAGTGGAAATTAAGAGAACTGTTGCTGCTCCTTCAGTTTTTATTT
TTCCACCTAGTGATGAGCAGCTGAAGAGTGGAACAGCATCTGTGGTGTGTCTTTT
GAATAATTTTTATCCTAGAGAAGCTAAGGTGCAGTGGAAAGTGGATAATGCATT
GCAGAGTGGAAATTCACAAGAATCAGTGACTGAGCAGGATTCAAAAGATAGTAC
ATATAGTCTTTCATCTACTTTGACACTGTCTAAGGCTGATTATGAGAAGCATAAA
GTGTATGCATGTGAGGTGACACATCAGGGGCTGTCTTCACCTGTGACAAAGTCTT
TTAATAGAGGGGAGTGTTGAACCTGA [symbol]
hIL-12-pembrolizumab (NotI-NheI)
>SEQ ID NO: 14 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCAGGAGAAA
GAGAGGATCCTCTGGAAGTGGAGCTACTAACTTCAGCCTGCTGAAGCAGGCTGG
AGATGTGGAGGAGAACCCTGGACCTATGGAGTTTGGGCTGAGCTGGGTTTTCCTT
GTTGCTCTTTTAAGAGGTGTCCAGTGTCAGGTGCAGTTGGTGCAGTCTGGAGTTG
AAGTGAAAAAGCCTGGTGCTTCAGTGAAGGTGAGTTGTAAGGCTTCAGGGTATA
CATTTACTAATTATTATATGTATTGGGTGAGACAGGCTCCTGGTCAGGGACTTGA
GTGGATGGGTGGAATTAATCCTTCTAATGGTGGAACTAATTTTAATGAGAAGTTT
AAGAATAGAGTGACTCTGACTACAGATAGTTCTACTACTACTGCTTATATGGAGC
TGAAGTCTCTGCAGTTTGATGATACAGCTGTGTATTATTGTGCTAGAAGAGATTA
TAGATTTGATATGGGATTTGATTATTGGGGTCAGGGGACAACAGTTACAGTTAGT
TCAGCTTCTACTAAAGGACCATCAGTTTTTCCTCTGGCACCATGTTCTAGGAGTA
CATCAGAGTCTACTGCTGCACTTGGGTGTTTGGTGAAAGATTATTTTCCAGAACC
TGTTACAGTGAGTTGGAATAGTGGAGCTCTGACATCAGGGGTTCATACTTTTCCT
GCTGTGTTGCAGTCATCTGGGCTGTATTCTCTGTCATCTGTTGTGACAGTGCCAAG
TAGTTCATTGGGAACTAAAACTTATACATGTAATGTGGATCATAAGCCTTCTAAT
ACTAAAGTGGATAAGAGGGTGGAATCTAAGTATGGACCACCATGTCCTCCATGT
CCAGCACCTGAATTTCTGGGAGGACCATCTGTGTTTTTGTTTCCACCAAAACCAA
AAGATACATTGATGATTTCAAGGACACCAGAGGTGACATGTGTGGTGGTGGATG
TGAGTCAGGAAGATCCTGAAGTGCAATTTAATTGGTATGTGGATGGAGTGGAGG
TTCATAATGCTAAAACTAAGCCTAGGGAAGAGCAGTTTAATAGTACATATAGGG
TGGTGTCTGTGCTTACAGTTCTGCATCAAGATTGGCTGAATGGAAAAGAGTATAA
GTGTAAAGTTAGTAATAAAGGGCTGCCTTCTTCAATTGAGAAAACAATTAGTAAG
GCAAAGGGTCAGCCTAGAGAGCCTCAAGTTTATACATTGCCACCTTCTCAGGAA
GAGATGACAAAGAATCAGGTGTCTCTGACATGTTTGGTTAAGGGTTTTTATCCAT
CAGATATTGCTGTGGAGTGGGAGTCAAATGGTCAACCAGAGAATAATTATAAAA
CTACACCACCAGTGCTGGATTCAGATGGGTCATTTTTTCTGTATAGTAGACTGAC
TGTGGATAAATCAAGGTGGCAGGAGGGAAATGTGTTTTCTTGTTCTGTGATGCAT
GAAGCTCTGCATAATCATTATACACAGAAATCATTGAGTCTGTCATTGGGTAAGA
GAAGGAAGAGGGGAAGTGGAGAGGGCAGAGGAAGTCTGCTAACATGTGGTGAT
GTGGAGGAGAATCCTGGACCTATGGACATGAGGGTCCCTGCTCAGCTCCTGGGG
CTCCTGCTGCTCTGGCTCTCAGGTGCCAGATGTGAAATTGTGCTGACTCAGAGTC
CTGCTACACTGTCATTGAGTCCAGGGGAAAGAGCAACACTGTCTTGTAGGGCAA
GTAAGGGAGTTTCAACTTCTGGTTATTCATATCTGCATTGGTATCAGCAGAAACC
AGGGCAGGCACCTAGGTTGCTGATTTATCTGGCATCATATTTGGAGAGTGGGGTT
CCTGCAAGATTTTCTGGATCTGGGTCAGGAACAGATTTTACACTGACAATTTCAA
GTCTTGAGCCTGAGGATTTTGCAGTTTATTATTGTCAGCATTCAAGAGATCTGCCT
CTGACTTTTGGAGGAGGTACAAAGGTTGAGATTAAAAGAACTGTGGCAGCACCT
TCAGTGTTTATTTTTCCTCCTAGTGATGAGCAATTGAAAAGTGGTACAGCATCTGT
TGTGTGTCTGCTTAATAATTTTTATCCTAGAGAGGCAAAAGTTCAGTGGAAGGTT
GATAATGCATTGCAATCTGGGAATTCTCAAGAGAGTGTTACAGAACAGGATTCA
AAAGATTCTACTTATTCACTGTCATCAACTCTGACACTGTCAAAGGCAGATTATG
AGAAGCATAAAGTGTATGCTTGTGAGGTGACTCATCAAGGGCTTAGTTCTCCTGT
TACTAAAAGTTTTAATAGAGGTGAGTGTTGAACCTGA [symbol]
hIL12-nivolumab (NotI-NheI)
>SEQ ID NO: 15 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCAGGAGAAA
GAGAGGATCCTCTGGAAGTGGAGCTACTAACTTCAGCCTGCTGAAGCAGGCTGG
AGATGTGGAGGAGAACCCTGGACCTATGGAGTTTGGGCTGAGCTGGGTTTTCCTT
GTTGCTCTTTTAAGAGGTGTCCAGTGTCAGGTGCAGCTGGTGGAGAGTGGTGGGG
GGGTGGTGCAACCTGGGAGGAGTCTGAGACTGGATTGTAAGGCTAGTGGGATTA
CTTTTTCAAATAGTGGAATGCATTGGGTGAGACAGGCTCCTGGGAAGGGGCTTGA
GTGGGTTGCTGTGATTTGGTATGATGGGTCTAAAAGGTATTATGCTGATAGTGTG
AAGGGTAGATTTACAATTTCTAGGGATAATAGTAAAAATACTCTGTTTCTTCAGA
TGAATTCTTTGAGAGCAGAGGATACAGCAGTTTATTATTGTGCAACAAATGATGA
TTATTGGGGGCAGGGTACTCTGGTTACTGTGTCTTCTGCTTCTACAAAGGGGCCA
TCAGTGTTTCCTCTGGCACCTTGTAGTAGATCAACTAGTGAGAGTACAGCTGCTC
TGGGGTGTCTTGTGAAAGATTATTTTCCTGAACCTGTGACTGTGTCTTGGAATTCT
GGAGCACTTACTTCAGGTGTTCATACATTTCCAGCAGTGCTGCAGAGTTCTGGGC
TGTATAGTCTGTCTTCAGTGGTGACAGTGCCTTCATCAAGTCTGGGAACAAAAAC
TTATACATGTAATGTGGATCATAAGCCATCAAATACTAAGGTGGATAAGAGAGT
GGAATCTAAGTATGGTCCACCATGTCCTCCTTGTCCAGCTCCTGAATTTCTGGGG
GGACCTAGTGTGTTTTTGTTTCCACCTAAGCCTAAGGATACACTTATGATTTCAAG
AACTCCTGAGGTTACTTGTGTGGTGGTGGATGTGTCTCAGGAAGATCCAGAAGTG
CAATTTAATTGGTATGTGGATGGGGTTGAAGTGCATAATGCAAAAACAAAACCA
AGGGAGGAGCAGTTTAATTCTACTTATAGGGTGGTGTCTGTGCTTACAGTGCTGC
ATCAAGATTGGTTGAATGGGAAAGAATATAAGTGTAAGGTTTCTAATAAGGGGT
TGCCTTCTAGTATTGAGAAGACTATTTCTAAGGCAAAGGGGCAGCCTAGAGAAC
CTCAAGTTTATACACTTCCTCCAAGTCAGGAGGAGATGACTAAAAATCAGGTTTC
ACTGACATGTCTGGTGAAAGGATTTTATCCATCAGATATTGCAGTTGAGTGGGAA
TCTAATGGGCAGCCTGAGAATAATTATAAGACTACACCACCTGTGCTTGATTCTG
ATGGAAGTTTTTTTCTGTATAGTAGACTGACAGTGGATAAAAGTAGATGGCAGGA
AGGTAATGTGTTTTCTTGTTCTGTGATGCATGAGGCACTGCATAATCATTATACTC
AAAAGAGTCTGTCTCTGTCTCTTGGAAAGAGAAGGAAGAGGGGAAGTGGAGAGG
GCAGAGGAAGTCTGCTAACATGTGGTGATGTGGAGGAGAATCCTGGACCTATGG
ACATGAGGGTCCCTGCTCAGCTCCTGGGGCTCCTGCTGCTCTGGCTCTCAGGTGC
CAGATGTGAGATTGTGCTGACACAGTCTCCTGCAACTCTGTCTCTGTCACCTGGG
GAGAGGGCTACTCTGTCATGTAGGGCTAGTCAGTCTGTGTCATCATATCTGGCAT
GGTATCAGCAAAAACCAGGTCAAGCTCCAAGGCTGCTGATTTATGATGCATCAA
ATAGGGCAACTGGTATTCCAGCAAGGTTTTCTGGGTCAGGAAGTGGAACAGATTT
TACACTGACTATTAGTTCTCTGGAGCCAGAGGATTTTGCAGTGTATTATTGTCAA
CAGAGTTCTAATTGGCCAAGAACATTTGGGCAGGGTACAAAAGTGGAGATTAAA
AGGACAGTGGCTGCTCCTTCTGTGTTTATTTTTCCACCTTCAGATGAACAACTTAA
AAGTGGTACAGCATCAGTGGTGTGTCTGTTGAATAATTTTTATCCAAGGGAAGCT
AAAGTTCAGTGGAAAGTTGATAATGCACTGCAGTCTGGGAATTCTCAGGAATCTG
TTACAGAACAGGATTCAAAAGATTCAACTTATTCTCTTTCTAGTACTCTGACATTG
TCTAAGGCTGATTATGAAAAGCATAAGGTGTATGCTTGTGAGGTGACACATCAG
GGACTTAGTTCACCAGTGACTAAATCTTTTAATAGGGGAGAGTGTTGAACCTGA

[symbol]

[symbol]

hIL12-bevacizumab (NotI-NheI)
>SEQ ID NO: 16 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCAGGAGAAA
GAGAGGATCCTCTGGAAGTGGAGCTACTAACTTCAGCCTGCTGAAGCAGGCTGG
AGATGTGGAGGAGAACCCTGGACCTATGGAGTTTGGGCTGAGCTGGGTTTTCCTT
GTTGCTCTTTTAAGAGGTGTCCAGTGTGAAGTGCAGCTTGTGGAGTCAGGAGGGG
GGCTGGTGCAGCCTGGGGGGAGTCTGAGGCTGAGTTGTGCAGCAAGTGGTTATA
CTTTTACAAATTATGGAATGAATTGGGTGAGACAGGCTCCTGGTAAAGGGCTGG
AGTGGGTTGGGTGGATTAATACTTATACAGGGGAGCCAACATATGCTGCAGATTT
TAAAAGGAGGTTTACTTTTAGTCTGGATACATCTAAGTCAACAGCTTATCTTCAG
ATGAATTCTCTTAGGGCTGAGGATACAGCTGTTTATTATTGTGCAAAGTATCCTC
ATTATTATGGATCATCTCATTGGTATTTTGATGTGTGGGGTCAGGGAACACTGGT
GACTGTTAGTAGTGCTAGTACTAAAGGGCCTTCAGTGTTTCCACTTGCTCCATCA
AGTAAGTCAACATCTGGAGGGACTGCTGCACTGGGGTGTTTGGTGAAGGATTATT
TTCCAGAACCAGTGACTGTTTCTTGGAATTCTGGAGCACTTACTTCTGGTGTGCAT
ACATTTCCTGCAGTGTTGCAGTCATCAGGATTGTATTCACTGTCTTCTGTGGTGAC
TGTGCCATCAAGTTCACTGGGAACACAGACATATATTTGTAATGTTAATCATAAA
CCTTCTAATACAAAGGTGGATAAGAAGGTGGAACCTAAATCTTGTGATAAAACA
CATACTTGTCCACCTTGTCCAGCTCCAGAACTGCTTGGGGGTCCATCTGTGTTTCT
TTTTCCTCCTAAGCCTAAAGATACACTTATGATTTCTAGAACACCAGAAGTTACT
TGTGTGGTGGTGGATGTGAGTCATGAGGACCCAGAAGTTAAGTTTAATTGGTATG
TGGATGGGGTTGAAGTGCATAATGCTAAAACAAAGCCTAGAGAAGAACAGTATA
ATAGTACATATAGAGTGGTGTCTGTGCTGACTGTGCTGCATCAGGATTGGCTGAA
TGGAAAGGAATATAAATGTAAGGTGAGTAATAAAGCTCTTCCAGCTCCTATTGA
GAAGACAATTTCTAAGGCTAAGGGGCAACCAAGGGAACCACAAGTGTATACATT
GCCACCTTCAAGGGAGGAGATGACTAAGAATCAGGTGTCTCTGACTTGTCTTGTT
AAAGGGTTTTATCCTAGTGATATTGCTGTGGAGTGGGAGTCAAATGGACAGCCA
GAAAATAATTATAAAACAACACCACCTGTGCTGGATAGTGATGGAAGTTTTTTTC
TGTATTCTAAGCTGACAGTGGATAAGAGTAGATGGCAGCAGGGTAATGTGTTTA
GTTGTAGTGTTATGCATGAAGCACTGCATAATCATTATACACAGAAATCTCTTTC
TCTGTCACCAGGGAAAAGAAGGAAGAGGGGAAGTGGAGAGGGCAGAGGAAGTC
TGCTAACATGTGGTGATGTGGAGGAGAATCCTGGACCTATGGACATGAGGGTCC
CTGCTCAGCTCCTGGGGCTCCTGCTGCTCTGGCTCTCAGGTGCCAGATGTGATAT
TCAGATGACACAGTCACCAAGTTCTCTTAGTGCTTCTGTGGGGGATAGAGTTACA
ATTACTTGTTCAGCAAGTCAGGATATTAGTAATTATCTTAATTGGTATCAGCAGA
AGCCTGGAAAGGCTCCTAAGGTGTTGATTTATTTTACTAGTTCACTGCATTCTGGT
GTTCCTAGTAGGTTTAGTGGGTCTGGATCAGGAACAGATTTTACACTGACAATTT
CATCACTGCAGCCTGAAGATTTTGCTACTTATTATTGTCAGCAGTATAGTACTGTT
CCTTGGACATTTGGGCAGGGTACAAAGGTGGAGATTAAAAGAACTGTGGCTGCA
CCTAGTGTTTTTATTTTTCCTCCTTCAGATGAGCAGCTGAAATCTGGTACAGCATC
TGTTGTTTGTCTGCTTAATAATTTTTATCCTAGGGAGGCAAAGGTGCAATGGAAG
GTGGATAATGCACTGCAGAGTGGAAATTCTCAAGAATCAGTGACTGAGCAAGAT
TCTAAAGATTCAACTTATTCTCTGAGTTCAACTCTTACTCTGTCTAAGGCTGATTA
TGAAAAACATAAGGTTTATGCTTGTGAGGTGACTCATCAAGGACTTAGTAGTCCT
GTGACAAAGAGTTTTAATAGGGGGGAGTGTTGAACCTGA [symbol]
hIL12-blinatumomab (NotI-NheI)
>SEQ ID NO: 17 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCAGGAGAAA
GAGAGGATCCTCTGGAAGTGGAGCTACTAACTTCAGCCTGCTGAAGCAGGCTGG
AGATGTGGAGGAGAACCCTGGACCTATGGACATGAGGGTCCCTGCTCAGCTCCT
GGGGCTCCTGCTGCTCTGGCTCTCAGGTGCCAGATGTGATATTCAGCTGACACAG
AGTCCTGCAAGTCTGGCTGTTAGTCTGGGGCAAAGAGCAACAATTAGTTGTAAG
GCTTCTCAGTCAGTGGATTATGATGGAGATAGTTATCTGAATTGGTATCAGCAGA
TTCCTGGGCAGCCTCCTAAGCTTCTGATTTATGATGCATCAAATCTTGTGTCAGG
AATTCCACCAAGGTTTTCTGGATCTGGAAGTGGAACTGATTTTACTCTGAATATT
CATCCTGTGGAAAAAGTGGATGCTGCAACATATCATTGTCAGCAGTCAACTGAG
GACCCTTGGACATTTGGAGGGGGGACAAAGCTTGAGATTAAGGGGGGGGGAGG
ATCAGGAGGGGGAGGTTCTGGAGGGGGAGGATCTCAGGTGCAGCTGCAGCAGTC
TGGGGCTGAGCTTGTTAGACCAGGATCTTCTGTGAAAATTTCATGTAAAGCATCA
GGGTATGCTTTTAGTTCTTATTGGATGAATTGGGTGAAACAGAGGCCTGGTCAGG
GACTGGAGTGGATTGGACAGATTTGGCCTGGGGATGGTGATACTAATTATAATG
GAAAGTTTAAAGGAAAAGCTACACTGACAGCAGATGAGTCTTCATCTACTGCAT
ATATGCAGCTTAGTTCTCTGGCAAGTGAGGATTCAGCAGTGTATTTTTGTGCAAG
AAGGGAGACTACAACAGTGGGAAGATATTATTATGCTATGGATTATTGGGGACA
AGGAACAACTGTGACAGTGTCTTCTGGGGGGGGTGGGTCTGATATTAAACTTCAG
CAATCAGGAGCAGAGCTTGCAAGGCCAGGTGCTTCAGTGAAAATGTCATGTAAG
ACTAGTGGGTATACATTTACTAGGTATACTATGCATTGGGTGAAACAAAGACCAG
GACAGGGGCTTGAGTGGATTGGATATATTAATCCAAGTAGGGGATATACAAATT
ATAATCAAAAGTTTAAAGATAAGGCTACTCTGACTACTGATAAGTCAAGTTCTAC
TGCTTATATGCAGCTTTCTTCTTTGACTTCAGAGGATTCAGCAGTGTATTATTGTG
CAAGATATTATGATGATCATTATTGTCTGGATTATTGGGGACAAGGAACAACACT
GACTGTGTCTTCTGTGGAGGGAGGGAGTGGAGGATCAGGTGGGTCAGGAGGTAG
TGGAGGGGTGGATGATATTCAACTGACACAGTCTCCAGCTATTATGAGTGCATCA
CCAGGGGAGAAGGTGACAATGACTTGTAGAGCATCAAGTTCTGTTTCTTATATGA
ATTGGTATCAGCAGAAGTCTGGGACAAGTCCTAAAAGATGGATTTATGATACTTC
TAAAGTGGCATCTGGAGTGCCTTATAGGTTTAGTGGATCTGGATCTGGAACATCT
TATTCATTGACTATTAGTAGTATGGAAGCAGAAGATGCAGCAACTTATTATTGTC
AGCAGTGGTCATCAAATCCTCTTACATTTGGAGCTGGGACTAAGTTGGAATTGAA
ACATCATCATCATCATCATTGAACCTGA [symbol]
hIL12-ranibizumab (NotI-NheI)
>SEQ ID NO: 18 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCAGGAGAAA
GAGAGGATCCTCTGGAAGTGGAGCTACTAACTTCAGCCTGCTGAAGCAGGCTGG
AGATGTGGAGGAGAACCCTGGACCTATGGAGTTTGGGCTGAGCTGGGTTTTCCTT
GTTGCTCTTTTAAGAGGTGTCCAGTGTGAAGTGCAGCTTGTGGAGTCAGGAGGGG
GGCTGGTGCAGCCTGGGGGGAGTCTGAGGCTGAGTTGTGCAGCAAGTGGTTATA
CTTTTACAAATTATGGAATGAATTGGGTGAGACAGGCTCCTGGTAAAGGGCTGG
AGTGGGTTGGGTGGATTAATACTTATACAGGGGAGCCAACATATGCTGCAGATTT
TAAAAGGAGGTTTACTTTTAGTCTGGATACATCTAAGTCAACAGCTTATCTTCAG
ATGAATTCTCTTAGGGCTGAGGATACAGCTGTTTATTATTGTGCAAAGTATCCTC
ATTATTATGGATCATCTCATTGGTATTTTGATGTGTGGGGTCAGGGAACACTGGT
GACTGTTAGTAGTGCTAGTACTAAAGGGCCTTCAGTGTTTCCACTTGCTCCATCA
AGTAAGTCAACATCTGGAGGGACTGCTGCACTGGGGTGTTTGGTGAAGGATTATT
TTCCAGAACCAGTGACTGTTTCTTGGAATTCTGGAGCACTTACTTCTGGTGTGCAT
ACATTTCCTGCAGTGTTGCAGTCATCAGGATTGTATTCACTGTCTTCTGTGGTGAC
TGTGCCATCAAGTTCACTGGGAACACAGACATATATTTGTAATGTTAATCATAAA
CCTTCTAATACAAAGGTGGATAAGAAGGTGGAACCTAAATCTTGTGATAAAACA
CATACTCTGAGAAGGAAGAGGGGAAGTGGAGAGGGCAGAGGAAGTCTGCTAAC
ATGTGGTGATGTGGAGGAGAATCCTGGACCTATGGACATGAGGGTCCCTGCTCA
GCTCCTGGGGCTCCTGCTGCTCTGGCTCTCAGGTGCCAGATGTGATATTCAGCTG
ACACAGTCACCAAGTTCTCTTAGTGCTTCTGTGGGGGATAGAGTTACAATTACTT
GTTCAGCAAGTCAGGATATTAGTAATTATCTTAATTGGTATCAGCAGAAGCCTGG
AAAGGCTCCTAAGGTGTTGATTTATTTTACTAGTTCACTGCATTCTGGTGTTCCTA
GTAGGTTTAGTGGGTCTGGATCAGGAACAGATTTTACACTGACAATTTCATCACT
GCAGCCTGAAGATTTTGCTACTTATTATTGTCAGCAGTATAGTACTGTTCCTTGGA
CATTTGGGCAGGGTACAAAGGTGGAGATTAAAAGAACTGTGGCTGCACCTAGTG
TTTTTATTTTTCCTCCTTCAGATGAGCAGCTGAAATCTGGTACAGCATCTGTTGTT
TGTCTGCTTAATAATTTTTATCCTAGGGAGGCAAAGGTGCAATGGAAGGTGGATA
ATGCACTGCAGAGTGGAAATTCTCAAGAATCAGTGACTGAGCAAGATTCTAAAG
ATTCAACTTATTCTCTGAGTTCAACTCTTACTCTGTCTAAGGCTGATTATGAAAAA
CATAAGGTTTATGCTTGTGAGGTGACTCATCAAGGACTTAGTAGTCCTGTGACAA
AGAGTTTTAATAGGGGGGAGTGTTGAACCTGA [symbol]
mIL12-iTME (HindIII-NheI)
>SEQ ID NO: 19 [symbol]
GGCATTCCGGTACTGTTGGTAAAGCCACCATGTGTCCTCAGAAGCTCAC
CATCTCCTGGTTTGCCATTGTTTTGCTGGTGTCTCCACTCATGGCCATGTGGGAGC
TGGAGAAAGATGTTTATGTTGTGGAGGTGGACTGGACTCCTGATGCCCCTGGAGA
AACAGTGAACCTCACCTGTGACACCCCTGAAGAAGATGACATCACCTGGACCTC
AGACCAGAGACATGGAGTCATAGGCTCTGGAAAGACCCTGACCATCACTGTCAA
AGAGTTTCTGGATGCTGGCCAGTACACCTGCCACAAAGGAGGGGAGACTCTGAG
CCACTCACATCTGCTGCTCCACAAGAAGGAAAATGGAATTTGGTCCACTGAAATT
CTGAAAAATTTCAAAAACAAGACTTTCCTGAAGTGTGAAGCACCAAATTACTCTG
GAAGGTTCACCTGCTCATGGCTGGTGCAAAGAAACATGGACTTGAAGTTCAACA
TCAAGAGCAGTAGCAGTTCCCCTGACTCTAGGGCAGTGACATGTGGAATGGCCT
CTCTGTCTGCAGAGAAGGTCACACTGGACCAAAGGGACTATGAGAAGTATTCAG
TGTCCTGCCAGGAGGATGTCACCTGCCCAACTGCTGAGGAGACCCTGCCCATTGA
ACTGGCCTTGGAAGCAAGGCAGCAGAATAAATATGAGAACTACAGCACCAGCTT
CTTCATCAGGGACATCATCAAACCAGACCCTCCCAAGAACTTGCAGATGAAGCC
TTTGAAGAACTCACAGGTGGAGGTCAGCTGGGAGTACCCTGACTCCTGGAGCAC
TCCCCATTCCTACTTCTCCCTCAAGTTCTTTGTTAGAATCCAGAGGAAGAAAGAA
AAGATGAAGGAGACAGAGGAGGGGTGTAACCAGAAAGGTGCCTTCCTGGTGGA
GAAGACATCTACAGAAGTCCAATGCAAAGGAGGGAATGTCTGTGTGCAAGCTCA
GGATAGGTATTACAATTCCTCATGCAGCAAGTGGGCATGTGTTCCCTGCAGGGTC
AGATCTGGTGGAGGTGGAAGTGGAGGTGGTGGATCTGGGGGTGGAGGCAGCAG
GGTCATTCCAGTCTCTGGACCTGCCAGGTGTCTTAGCCAGTCCAGAAACCTGCTG
AAGACCACAGATGACATGGTGAAGACTGCCAGAGAAAAACTGAAACATTATTCC
TGCACTGCTGAAGACATTGATCATGAAGACATCACAAGGGACCAAACCAGCACA
TTGAAGACCTGTCTGCCACTGGAACTGCACAAGAATGAGAGTTGCCTGGCTACTA
GAGAGACTTCTTCCACAACAAGAGGGAGCTGCCTGCCCCCACAGAAGACCTCTT
TGATGATGACCCTGTGCCTTGGTAGCATCTATGAGGACTTGAAGATGTACCAGAC
AGAGTTCCAGGCCATCAATGCAGCACTTCAGAATCACAACCATCAGCAGATCAT
TCTGGACAAGGGCATGCTGGTGGCCATTGATGAGCTGATGCAGTCTCTGAATCAT
AATGGAGAGACTCTGAGACAGAAACCTCCTGTGGGAGAAGCAGACCCTTACAGA
GTGAAAATGAAGCTCTGCATCCTGCTTCATGCCTTCAGCACCAGAGTGGTGACCA
TCAACAGGGTGATGGGCTATCTGAGCTCTGCCAGAAGGAAGAGGGGATCCTCTG
GAAGTGGAGCTACTAACTTCAGCCTGCTGAAGCAGGCTGGAGATGTGGAGGAGA
ACCCTGGACCTATGATGGTGTTAAGTCTTCTGTACCTGTTGACAGCCCTTCCTGGT
ATCCTGTCAGAGGTGCAGCTGCAGGAGTCAGGACCAGGCCTGGTGAAACCTTCT
CAGAGTCTGTCCCTGACTTGTTCTGTCACTGGGTATTCAATTACATCTTCATATAG
ATGGAACTGGATCAGGAAGTTTCCAGGGAATAGGCTGGAGTGGATGGGGTACAT
AAATTCAGCTGGTATTTCTAATTACAATCCATCTCTGAAGAGAAGAATCTCCATC
ACAAGAGACACATCCAAAAACCAGTTCTTTCTGCAGGTTAATTCTGTGACTACTG
AGGATGCTGCCACATATTACTGTGCAAGAAGTGATAATATGGGGACAACACCTT
TTACTTATTGGGGTCAAGGGACATTGGTGACTGTGAGTTCTGCATCAACAACAGC
ACCATCTGTCTATCCACTGGCCCCTGTGTGTGGAGATACAACTGGCTCCTCAGTG
ACTCTGGGATGCCTGGTCAAGGGTTATTTCCCTGAGCCAGTGACCTTGACCTGGA
ACTCTGGCTCCCTGTCCAGTGGTGTGCACACCTTCCCAGCTGTCCTGCAGTCTGA
CCTCTACACCCTCAGCAGCTCAGTGACTGTAACCTCTAGCACCTGGCCCAGCCAG
TCCATCACCTGCAATGTGGCCCACCCAGCAAGCAGCACCAAGGTGGACAAGAAA
ATTGAGCCCAGAGGGCCCACAATCAAGCCCTGTCCTCCATGCAAATGCCCAGCA
CCTAATGCAGCTGGTGGACCATCTGTCTTCATCTTCCCTCCAAAGATCAAGGATG
TACTCATGATCTCCCTGAGCCCCATAGTCACATGTGTGGTGGTGGATGTGAGTGA
GGATGACCCAGATGTCCAGATCAGCTGGTTTGTGAACAATGTGGAAGTACACAC
AGCTCAGACACAAACCCATAGAGAGGATTACAACAGTACTCTCAGGGTGGTCAG
TGCCCTCCCCATCCAGCACCAGGACTGGATGAGTGGCAAGGAGTTCAAATGCAA
GGTCAACAACAAAGACCTGGGGGCACCCATTGAGAGAACCATCTCAAAACCCAA
AGGGTCAGTAAGAGCTCCACAGGTATATGTCTTGCCTCCACCAGAAGAAGAGAT
GACTAAGAAACAGGTCACTCTGACCTGCATGGTCACAGACTTCATGCCTGAAGA
CATTTATGTGGAGTGGACCAACAATGGGAAAACAGAGCTGAACTACAAGAACAC
TGAACCAGTCCTGGACTCTGATGGTTCTTACTTCATGTACAGCAAGCTGAGAGTG
GAAAAGAAGAACTGGGTGGAAAGAAATAGCTACTCCTGTTCAGTGGTCCATGAG
GGTCTGCACAATCACCACACAACTAAGAGCTTCTCTAGGACTCCAAGAAGGAAG
AGGGGAAGTGGAGAGGGCAGAGGAAGTCTGCTAACATGTGGTGATGTGGAGGA
GAATCCTGGACCTATGAGGTGCCTAGCTGAGTTCCTGGGGCTGCTTGTGCTCTGG
ATTCCTGGAGCCATTGGGGATATTGTGATGACTCAGGGTACTCTGCCTAATCCTG
TGCCAAGTGGGGAGTCTGTGTCTATTACATGTAGGAGTTCAAAGAGTCTTCTTTA
TTCAGATGGAAAAACATATCTGAATTGGTATCTGCAGAGACCTGGGCAGAGTCC
TCAGCTGCTGATTTATTGGATGTCTACTAGGGCATCTGGGGTGTCTGATAGATTTT
CTGGTAGTGGTAGTGGTACAGATTTTACATTGAAGATTTCTGGGGTGGAGGCTGA
AGATGTGGGTATTTATTATTGTCAGCAAGGTCTGGAGTTTCCAACATTTGGGGGA
GGTACTAAGCTGGAGCTGAAGAGAACTGATGCTGCACCAACTGTATCCATCTTCC
CACCATCCAGTGAGCAGCTGACATCTGGAGGTGCCTCAGTTGTGTGCTTCCTGAA
CAACTTCTACCCCAAAGACATCAATGTCAAGTGGAAGATTGATGGCAGTGAAAG
ACAAAATGGGGTCCTGAACAGTTGGACTGATCAGGACAGCAAAGACAGCACCTA
CAGCATGAGCAGCACCCTCACCCTGACCAAGGATGAGTATGAAAGACATAACAG
CTATACCTGTGAGGCCACTCACAAGACATCAACTTCACCCATTGTCAAGAGCTTC
AACAGGAATGAGTGTTGAACCTGA [symbol]
mIL2-mIL12 (HindIII-NheI)
>SEQ ID NO: 20 [symbol]
GGCATTCCGGTACTGTTGGTAAAGCCACCATGTACAGCATGCAGCTGGC
CTCCTGTGTGACACTGACACTGGTGCTGCTGGTGAACTCTGCACCCACTTCAAGC
TCCACCTCAAGCTCTACAGCTGAAGCCCAGCAGCAGCAGCAGCAGCAGCAGCAG
CAGCAGCAGCACCTGGAGCAGCTGCTGATGGACCTGCAGGAGCTGCTGAGCAGG
ATGGAGAATTACAGGAACCTGAAACTCCCCAGGATGCTGACCTTCAAATTTTACT
TGCCCAAGCAGGCCACAGAACTGAAGGATCTGCAGTGCCTGGAAGATGAACTTG
GACCTCTGAGGCATGTGCTGGATCTGACTCAAAGCAAGAGCTTTCAACTGGAAG
ATGCTGAGAATTTCATCAGCAATATCAGAGTGACTGTGGTCAAACTGAAGGGCT
CTGACAACACATTTGAGTGCCAATTTGATGATGAGTCAGCCACTGTGGTGGACTT
TCTGAGGAGATGGATTGCCTTCTGTCAAAGCATCATCTCAACAAGCCCTCAAAGA
AGGAAGAGGGGAAGTGGAGAGGGCAGAGGAAGTCTGCTAACATGTGGTGATGT
GGAGGAGAATCCTGGACCTATGTGTCCTCAGAAGCTCACCATCTCCTGGTTTGCC
ATTGTTTTGCTGGTGTCTCCACTCATGGCCATGTGGGAGCTGGAGAAAGATGTTT
ATGTTGTGGAGGTGGACTGGACTCCTGATGCCCCTGGAGAAACAGTGAACCTCA
CCTGTGACACCCCTGAAGAAGATGACATCACCTGGACCTCAGACCAGAGACATG
GAGTCATAGGCTCTGGAAAGACCCTGACCATCACTGTCAAAGAGTTTCTGGATGC
TGGCCAGTACACCTGCCACAAAGGAGGGGAGACTCTGAGCCACTCACATCTGCT
GCTCCACAAGAAGGAAAATGGAATTTGGTCCACTGAAATTCTGAAAAATTTCAA
AAACAAGACTTTCCTGAAGTGTGAAGCACCAAATTACTCTGGAAGGTTCACCTGC
TCATGGCTGGTGCAAAGAAACATGGACTTGAAGTTCAACATCAAGAGCAGTAGC
AGTTCCCCTGACTCTAGGGCAGTGACATGTGGAATGGCCTCTCTGTCTGCAGAGA
AGGTCACACTGGACCAAAGGGACTATGAGAAGTATTCAGTGTCCTGCCAGGAGG
ATGTCACCTGCCCAACTGCTGAGGAGACCCTGCCCATTGAACTGGCCTTGGAAGC
AAGGCAGCAGAATAAATATGAGAACTACAGCACCAGCTTCTTCATCAGGGACAT
CATCAAACCAGACCCTCCCAAGAACTTGCAGATGAAGCCTTTGAAGAACTCACA
GGTGGAGGTCAGCTGGGAGTACCCTGACTCCTGGAGCACTCCCCATTCCTACTTC
TCCCTCAAGTTCTTTGTTAGAATCCAGAGGAAGAAAGAAAAGATGAAGGAGACA
GAGGAGGGGTGTAACCAGAAAGGTGCCTTCCTGGTGGAGAAGACATCTACAGAA
GTCCAATGCAAAGGAGGGAATGTCTGTGTGCAAGCTCAGGATAGGTATTACAAT
TCCTCATGCAGCAAGTGGGCATGTGTTCCCTGCAGGGTCAGATCTGGTGGAGGTG
GAAGTGGAGGTGGTGGATCTGGGGGTGGAGGCAGCAGGGTCATTCCAGTCTCTG
GACCTGCCAGGTGTCTTAGCCAGTCCAGAAACCTGCTGAAGACCACAGATGACA
TGGTGAAGACTGCCAGAGAAAAACTGAAACATTATTCCTGCACTGCTGAAGACA
TTGATCATGAAGACATCACAAGGGACCAAACCAGCACATTGAAGACCTGTCTGC
CACTGGAACTGCACAAGAATGAGAGTTGCCTGGCTACTAGAGAGACTTCTTCCA
CAACAAGAGGGAGCTGCCTGCCCCCACAGAAGACCTCTTTGATGATGACCCTGT
GCCTTGGTAGCATCTATGAGGACTTGAAGATGTACCAGACAGAGTTCCAGGCCA
TCAATGCAGCACTTCAGAATCACAACCATCAGCAGATCATTCTGGACAAGGGCA
TGCTGGTGGCCATTGATGAGCTGATGCAGTCTCTGAATCATAATGGAGAGACTCT
GAGACAGAAACCTCCTGTGGGAGAAGCAGACCCTTACAGAGTGAAAATGAAGCT
CTGCATCCTGCTTCATGCCTTCAGCACCAGAGTGGTGACCATCAACAGGGTGATGGGCTA
TCTGAGCTCTGCCTAAACCTGA [symbol]
hIL12-durvalumab (NotI-NheI)
>SEQ ID NO: 21 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCAGGAGAAA
GAGAGGATCCTCTGGAAGTGGAGCTACTAACTTCAGCCTGCTGAAGCAGGCTGG
AGATGTGGAGGAGAACCCTGGACCTATGGAGTTTGGGCTGAGCTGGGTTTTCCTT
GTTGCTCTTTTAAGAGGTGTCCAGTGTGAGGTGCAGCTGGTGGAAAGTGGGGGG
GGACTTGTGCAGCCTGGGGGGTCACTTAGGCTTTCATGTGCTGCTTCTGGGTTTA
CATTTAGTAGATATTGGATGAGTTGGGTGAGGCAGGCACCAGGTAAGGGGCTGG
AGTGGGTGGCTAATATTAAGCAAGATGGTTCTGAGAAGTATTATGTGGATTCTGT
TAAGGGTAGGTTTACAATTTCTAGGGATAATGCTAAGAATAGTCTGTATCTGCAG
ATGAATTCACTTAGAGCAGAGGATACTGCAGTGTATTATTGTGCTAGAGAAGGG
GGTTGGTTTGGTGAATTGGCATTTGATTATTGGGGACAGGGGACTCTGGTTACAG
TGTCATCAGCAAGTACTAAGGGGCCATCTGTTTTTCCTCTGGCTCCTTCATCAAA
GAGTACAAGTGGAGGTACAGCTGCTCTTGGTTGTCTTGTGAAGGATTATTTTCCT
GAGCCTGTGACTGTGTCATGGAATTCAGGGGCTCTGACTAGTGGAGTGCATACTT
TTCCTGCTGTGCTGCAGAGTAGTGGACTGTATAGTCTGAGTTCTGTGGTGACAGT
GCCATCATCTAGTCTGGGAACACAAACATATATTTGTAATGTGAATCATAAACCA
TCTAATACAAAGGTTGATAAGAGAGTGGAGCCTAAAAGTTGTGATAAGACACAT
ACATGTCCACCATGTCCTGCTCCTGAATTTGAAGGTGGTCCAAGTGTTTTTCTGTT
TCCTCCTAAGCCTAAGGATACTCTTATGATTTCAAGGACTCCAGAAGTGACTTGT
GTGGTGGTTGATGTTAGTCATGAAGATCCTGAGGTTAAATTTAATTGGTATGTGG
ATGGAGTTGAAGTGCATAATGCAAAGACAAAACCAAGGGAAGAGCAGTATAATT
CTACATATAGGGTGGTTTCAGTGTTGACAGTGCTGCATCAAGATTGGCTGAATGG
AAAGGAATATAAATGTAAGGTTTCTAATAAAGCTCTGCCTGCTAGTATTGAAAAG
ACAATTTCAAAAGCAAAAGGACAACCAAGGGAACCACAGGTTTATACACTTCCT
CCTAGTAGGGAAGAAATGACAAAGAATCAGGTTAGTCTGACATGTCTGGTGAAA
GGGTTTTATCCTTCTGATATTGCAGTGGAATGGGAGTCAAATGGGCAGCCTGAAA
ATAATTATAAGACAACTCCACCAGTTCTTGATTCAGATGGATCTTTTTTTCTGTAT
AGTAAGCTGACAGTGGATAAATCTAGGTGGCAGCAAGGTAATGTGTTTAGTTGT
AGTGTTATGCATGAAGCACTGCATAATCATTATACTCAAAAGTCACTGAGTCTGT
CACCAGGGAAAAGAAGGAAGAGGGGAAGTGGAGAGGGCAGAGGAAGTCTGCTA
ACATGTGGTGATGTGGAGGAGAATCCTGGACCTATGGACATGAGGGTCCCTGCT
CAGCTCCTGGGGCTCCTGCTGCTCTGGCTCTCAGGTGCCAGATGTGAGATTGTTC
TGACACAGTCTCCTGGAACACTGTCACTGTCACCAGGAGAGAGGGCAACACTGT
CATGTAGAGCAAGTCAGAGGGTGAGTAGTAGTTATCTGGCTTGGTATCAGCAGA
AACCAGGGCAGGCACCTAGATTGCTTATTTATGATGCTTCAAGTAGGGCTACAGG
GATTCCTGATAGATTTTCAGGGAGTGGGTCAGGGACAGATTTTACATTGACAATT
AGTAGGTTGGAGCCTGAGGATTTTGCTGTGTATTATTGTCAGCAGTATGGATCTT
TGCCTTGGACATTTGGTCAGGGGACAAAAGTGGAGATTAAGAGGACAGTGGCAG
CTCCATCTGTGTTTATTTTTCCTCCTAGTGATGAGCAGCTTAAATCTGGGACAGCT
TCAGTGGTGTGTTTGCTTAATAATTTTTATCCAAGGGAGGCAAAGGTGCAGTGGA
AGGTTGATAATGCATTGCAGAGTGGAAATTCTCAGGAGAGTGTGACAGAGCAGG
ATTCTAAAGATTCAACATATTCTCTGTCTAGTACACTGACTCTGTCTAAGGCTGAT
TATGAAAAGCATAAGGTGTATGCATGTGAGGTTACACATCAAGGGCTGTCTTCTCCTGTG
ACAAAATCATTTAATAGAGGAGAATGTTGAACCTGA [symbol]
hIL12-atezolizumab (NotI-NheI)
>SEQ ID NO: 22 [symbol]
TCGAGATCTGCGATCTAAGTAAGCTTGGCATTCCGGTACTGTTGGTA
AAGCCACCATGTGTCACCAGCAGTTGGTCATCTCTTGGTTTTCCCTGGTTTTTCTG
GCATCTCCCCTGGTGGCCATCTGGGAACTGAAGAAAGATGTTTATGTGGTGGAAT
TGGATTGGTATCCTGATGCCCCTGGAGAAATGGTGGTCCTCACCTGTGACACCCC
TGAAGAAGATGGTATCACCTGGACCTTGGACCAGAGCAGTGAGGTCCTGGGCTC
TGGCAAAACCCTGACCATCCAAGTCAAAGAGTTTGGAGATGCTGGCCAGTACAC
CTGTCACAAAGGAGGGGAGGTTCTGAGCCATTCCCTCCTGCTGCTTCACAAAAAG
GAAGATGGAATTTGGTCCACTGATATTCTGAAGGACCAGAAAGAACCCAAAAAT
AAGACCTTTCTGAGATGTGAGGCCAAGAATTATTCTGGAAGATTCACCTGCTGGT
GGCTGACCACAATCAGTACTGATTTGACATTCAGTGTCAAAAGCAGCAGAGGCT
CTTCTGACCCCCAAGGGGTGACCTGTGGAGCTGCTACACTCTCTGCAGAGAGAGT
CAGAGGGGACAACAAGGAGTATGAGTACTCAGTGGAGTGCCAGGAGGACAGTG
CCTGCCCAGCTGCTGAGGAGAGTCTGCCCATTGAGGTCATGGTGGATGCTGTTCA
CAAGCTCAAGTATGAAAACTACACCAGCAGCTTCTTCATCAGGGACATCATCAA
ACCTGACCCACCCAAGAACTTGCAGCTGAAGCCACTGAAGAATTCTAGGCAGGT
GGAGGTCAGCTGGGAGTACCCTGACACCTGGAGTACTCCACATTCCTACTTCTCC
CTGACATTCTGTGTTCAGGTCCAGGGCAAGAGCAAGAGAGAAAAGAAAGATAGA
GTCTTCACAGACAAGACCTCAGCCACAGTCATCTGCAGGAAAAATGCCAGCATT
AGTGTGAGGGCCCAGGACAGATACTATAGCTCATCTTGGAGTGAATGGGCATCT
GTGCCCTGCAGTGGTGGAGGTGGAAGTGGAGGTGGTGGATCTGGGGGTGGAGGC
AGCAGAAACCTCCCTGTGGCCACTCCAGACCCAGGAATGTTCCCATGCCTTCACC
ACTCCCAAAACCTGCTGAGGGCTGTCAGCAACATGCTCCAGAAGGCCAGACAAA
CTCTGGAATTTTACCCTTGCACTTCTGAAGAGATTGATCATGAAGATATCACAAA
AGATAAAACCAGCACAGTGGAGGCCTGTCTGCCATTGGAACTCACCAAGAATGA
GAGTTGCCTGAATTCCAGAGAGACCTCTTTCATCACTAATGGGAGTTGCCTGGCC
TCCAGAAAGACCTCTTTTATGATGGCCCTGTGCCTTAGTAGTATTTATGAAGACTT
GAAGATGTACCAGGTGGAGTTCAAGACCATGAATGCAAAGCTTCTGATGGACCC
TAAGAGGCAGATCTTTCTGGATCAAAACATGCTGGCAGTTATTGATGAGCTGATG
CAGGCCCTGAATTTCAACAGTGAGACTGTGCCACAAAAATCCTCCCTTGAAGAA
CCTGATTTTTATAAAACTAAAATCAAGCTCTGCATCCTTCTTCATGCTTTCAGAAT
TAGGGCAGTGACTATTGACAGAGTGATGAGCTATCTGAATGCTTCCAGGAGAAA
GAGAGGATCCTCTGGAAGTGGAGCTACTAACTTCAGCCTGCTGAAGCAGGCTGG
AGATGTGGAGGAGAACCCTGGACCTATGGAGTTTGGGCTGAGCTGGGTTTTCCTT
GTTGCTCTTTTAAGAGGTGTCCAGTGTGAGGTGCAGCTTGTGGAATCTGGGGGGG
GGCTGGTGCAGCCTGGTGGTAGTCTGAGACTGTCATGTGCTGCTAGTGGGTTTAC
ATTTTCAGATTCTTGGATTCATTGGGTGAGACAGGCACCTGGGAAAGGTCTGGAG
TGGGTGGCATGGATTTCACCTTATGGGGGATCTACATATTATGCTGATAGTGTGA
AGGGGAGGTTTACAATTTCTGCTGATACATCTAAGAATACAGCTTATCTTCAGAT
GAATTCTCTGAGAGCTGAAGATACTGCAGTGTATTATTGTGCTAGGAGGCATTGG
CCTGGAGGGTTTGATTATTGGGGTCAAGGTACACTGGTGACTGTTAGTAGTGCTA
GTACTAAAGGTCCTTCTGTGTTTCCACTGGCACCAAGTTCAAAGAGTACATCAGG
AGGGACTGCAGCTCTGGGTTGTTTGGTGAAAGATTATTTTCCTGAACCTGTGACA
GTTTCATGGAATTCTGGAGCACTGACTTCTGGAGTGCATACATTTCCTGCTGTGCT
GCAGTCTAGTGGGTTGTATTCATTGTCAAGTGTGGTTACAGTGCCTTCAAGTTCTC
TGGGTACACAGACTTATATTTGTAATGTGAATCATAAGCCAAGTAATACAAAAGT
GGATAAGAAAGTTGAGCCTAAATCATGTGATAAAACTCATACTTGTCCACCTTGT
CCTGCTCCAGAGCTGTTGGGTGGGCCTAGTGTTTTTCTTTTTCCACCAAAGCCAA
AAGATACTTTGATGATTTCAAGGACACCAGAAGTGACATGTGTGGTTGTTGATGT
TTCTCATGAAGATCCTGAGGTGAAGTTTAATTGGTATGTTGATGGGGTTGAGGTG
CATAATGCTAAGACAAAACCTAGGGAGGAACAGTATGCTTCTACATATAGAGTT
GTGTCAGTGTTGACAGTGCTGCATCAAGATTGGCTTAATGGGAAAGAATATAAGT
GTAAGGTTTCAAATAAGGCATTGCCAGCTCCAATTGAAAAGACAATTTCTAAGG
CTAAGGGTCAGCCTAGGGAGCCACAGGTGTATACTCTGCCACCTTCAAGAGAGG
AAATGACTAAGAATCAGGTGTCATTGACATGTTTGGTGAAAGGATTTTATCCTTC
AGATATTGCTGTGGAATGGGAATCTAATGGACAACCAGAGAATAATTATAAAAC
TACTCCTCCTGTGCTGGATAGTGATGGAAGTTTTTTTCTGTATTCTAAACTTACTG
TTGATAAAAGTAGATGGCAGCAAGGTAATGTTTTTTCTTGTTCTGTGATGCATGA
AGCTCTTCATAATCATTATACTCAGAAGAGTCTGAGTCTGTCTCCTGGAAAGAGA
AGGAAGAGGGGAAGTGGAGAGGGCAGAGGAAGTCTGCTAACATGTGGTGATGT
GGAGGAGAATCCTGGACCTATGGACATGAGGGTCCCTGCTCAGCTCCTGGGGCT
CCTGCTGCTCTGGCTCTCAGGTGCCAGATGTGATATTCAGATGACACAGAGTCCA
AGTTCACTGTCAGCTTCTGTTGGTGATAGAGTTACTATTACATGTAGAGCTTCTCA
GGATGTGAGTACTGCAGTGGCTTGGTATCAGCAGAAGCCAGGGAAGGCTCCAAA
GCTGCTGATTTATTCAGCATCATTTCTGTATTCAGGGGTGCCATCAAGATTTTCAG
GTTCTGGAAGTGGAACAGATTTTACTCTGACTATTTCATCTCTGCAACCAGAAGA
TTTTGCAACATATTATTGTCAGCAGTATCTGTATCATCCAGCAACATTTGGTCAGG
GTACTAAAGTGGAAATTAAAAGGACAGTGGCAGCACCATCAGTTTTTATTTTTCC
ACCTAGTGATGAACAGCTGAAAAGTGGGACAGCTTCAGTGGTGTGTCTGCTTAAT
AATTTTTATCCTAGAGAAGCAAAAGTGCAGTGGAAGGTGGATAATGCACTGCAA
AGTGGGAATTCACAGGAATCAGTGACAGAGCAAGATTCTAAGGATTCTACATAT
AGTCTGTCTTCTACATTGACTCTGTCTAAGGCAGATTATGAAAAGCATAAAGTTT
ATGCATGTGAGGTTACTCATCAGGGATTGTCATCACCTGTTACTAAAAGTTTTAATAGGG
GTGAGTGTTGAACCTGA [symbol]
iTME (HindIII-NheI)
>SEQ ID NO: 23 [symbol]
GGCATTCCGGTACTGTTGGTAAAGCCACCATGATGGTGTTAAGTCTTCT
GTACCTGTTGACAGCCCTTCCTGGTATCCTGTCAGAGGTGCAGCTGCAGGAGTCA
GGACCAGGCCTGGTGAAACCTTCTCAGAGTCTGTCCCTGACTTGTTCTGTCACTG
GGTATTCAATTACATCTTCATATAGATGGAACTGGATCAGGAAGTTTCCAGGGAA
TAGGCTGGAGTGGATGGGGTACATAAATTCAGCTGGTATTTCTAATTACAATCCA
TCTCTGAAGAGAAGAATCTCCATCACAAGAGACACATCCAAAAACCAGTTCTTTC
TGCAGGTTAATTCTGTGACTACTGAGGATGCTGCCACATATTACTGTGCAAGAAG
TGATAATATGGGGACAACACCTTTTACTTATTGGGGTCAAGGGACATTGGTGACT
GTGAGTTCTGCATCAACAACAGCACCATCTGTCTATCCACTGGCCCCTGTGTGTG
GAGATACAACTGGCTCCTCAGTGACTCTGGGATGCCTGGTCAAGGGTTATTTCCC
TGAGCCAGTGACCTTGACCTGGAACTCTGGCTCCCTGTCCAGTGGTGTGCACACC
TTCCCAGCTGTCCTGCAGTCTGACCTCTACACCCTCAGCAGCTCAGTGACTGTAA
CCTCTAGCACCTGGCCCAGCCAGTCCATCACCTGCAATGTGGCCCACCCAGCAAG
CAGCACCAAGGTGGACAAGAAAATTGAGCCCAGAGGGCCCACAATCAAGCCCTG
TCCTCCATGCAAATGCCCAGCACCTAATGCAGCTGGTGGACCATCTGTCTTCATC
TTCCCTCCAAAGATCAAGGATGTACTCATGATCTCCCTGAGCCCCATAGTCACAT
GTGTGGTGGTGGATGTGAGTGAGGATGACCCAGATGTCCAGATCAGCTGGTTTGT
GAACAATGTGGAAGTACACACAGCTCAGACACAAACCCATAGAGAGGATTACAA
CAGTACTCTCAGGGTGGTCAGTGCCCTCCCCATCCAGCACCAGGACTGGATGAGT
GGCAAGGAGTTCAAATGCAAGGTCAACAACAAAGACCTGGGGGCACCCATTGAG
AGAACCATCTCAAAACCCAAAGGGTCAGTAAGAGCTCCACAGGTATATGTCTTG
CCTCCACCAGAAGAAGAGATGACTAAGAAACAGGTCACTCTGACCTGCATGGTC
ACAGACTTCATGCCTGAAGACATTTATGTGGAGTGGACCAACAATGGGAAAACA
GAGCTGAACTACAAGAACACTGAACCAGTCCTGGACTCTGATGGTTCTTACTTCA
TGTACAGCAAGCTGAGAGTGGAAAAGAAGAACTGGGTGGAAAGAAATAGCTAC
TCCTGTTCAGTGGTCCATGAGGGTCTGCACAATCACCACACAACTAAGAGCTTCT
CTAGGACTCCAAGAAGGAAGAGGGGAAGTGGAGAGGGCAGAGGAAGTCTGCTA
ACATGTGGTGATGTGGAGGAGAATCCTGGACCTATGAGGTGCCTAGCTGAGTTC
CTGGGGCTGCTTGTGCTCTGGATTCCTGGAGCCATTGGGGATATTGTGATGACTC
AGGGTACTCTGCCTAATCCTGTGCCAAGTGGGGAGTCTGTGTCTATTACATGTAG
GAGTTCAAAGAGTCTTCTTTATTCAGATGGAAAAACATATCTGAATTGGTATCTG
CAGAGACCTGGGCAGAGTCCTCAGCTGCTGATTTATTGGATGTCTACTAGGGCAT
CTGGGGTGTCTGATAGATTTTCTGGTAGTGGTAGTGGTACAGATTTTACATTGAA
GATTTCTGGGGTGGAGGCTGAAGATGTGGGTATTTATTATTGTCAGCAAGGTCTG
GAGTTTCCAACATTTGGGGGAGGTACTAAGCTGGAGCTGAAGAGAACTGATGCT
GCACCAACTGTATCCATCTTCCCACCATCCAGTGAGCAGCTGACATCTGGAGGTG
CCTCAGTTGTGTGCTTCCTGAACAACTTCTACCCCAAAGACATCAATGTCAAGTG
GAAGATTGATGGCAGTGAAAGACAAAATGGGGTCCTGAACAGTTGGACTGATCA
GGACAGCAAAGACAGCACCTACAGCATGAGCAGCACCCTCACCCTGACCAAGGA
TGAGTATGAAAGACATAACAGCTATACCTGTGAGGCCACTCACAAGACATCAAC
TTCACCCATTGTCAAGAGCTTCAACAGGAATGAGTGTTGAACCTGA

[symbol]

We claim:

1. A method for treating cancer in a subject in need thereof, comprising administering to the subject a nucleic acid construct comprising an expression cassette, wherein the expression cassette comprises a cancer-specific promoter and one or more therapeutic genes.

2. The method of claim 1, wherein the cancer-specific promoter is the PEG-3 promoter.

3. The method of claim 1, wherein the one or more therapeutic genes is a cytokine, a thymidine kinase, a toxin, a pathogen-associated molecular pattern (PAMP), a danger-associated molecular pattern (DAMP), an immune checkpoint inhibitor gene, or any combination thereof.

4. The method of claim 3, wherein the thymidine kinase is HSV1-TK.

5. The method of claim 3, wherein the PAMP is flagellin (FliC).

6. The method of claim 3, wherein the cytokine is a single chain variant of IL-12 (scIL-12).

7. The method of claim 1, wherein if multiple therapeutic genes are present, the multiple therapeutic genes are separated by a picornavirus 2A ribosome skipping sequence.

8. The method of claim 7, wherein the picornavirus ribosome skipping sequence is P2A or T2A.

9. The method of claim 1, wherein the therapeutic gene is engineered to have a reduced CpG content compared to its wild-type counterpart.

10. The method of claim 1, wherein the nucleic acid construct comprises a CpG-free plasmid backbone.

11. The method of claim 1, wherein the nucleic acid construct is formulated into nanoparticles with a cationic polymer.

12. The method of claim 11, wherein the nanoparticles are prepared at a N/P ratio of 4 or 6.

13. The method of claim 11, wherein the nanoparticles are lyophilized.

14. The method of claim 1, wherein the nucleic acid construct is delivered systemically.

15. The method of claim 1, wherein the cancer is selected from the group consisting of breast cancer, melanoma, carcinoma of unknown primary (CUP), neuroblastoma, malignant glioma, cervical cancer, colon cancer, hepatocarcinoma, ovarian cancer, lung cancer, pancreatic cancer, and prostate cancer.

16. The method of claim 3, wherein the immune checkpoint inhibitor gene encodes a monoclonal antibody selected from the group consisting of an anti-PD-1 antibody, an anti-PD-L1 antibody, and an anti-CTLA-4 antibody.

17. The method of claim 3, wherein the immune checkpoint inhibitor gene encodes an immune checkpoint inhibitor fusion protein comprising a PD-1 fusion protein.

18. The method of claim 17, wherein the PD-1 fusion protein comprises a fusion of PD-1 and an immunoglobulin Fc region.

19. The method of claim 3, wherein the cytokine is selected from the group consisting of IL-12, IL-24, IL-2, IL-15, and GM-CSF.

20. A nucleic acid construct for the treatment of cancer comprising an expression cassette, wherein the expression cassette comprises a cancer-specific promoter and one or more therapeutic genes.

21. The nucleic acid construct of claim 20, wherein the cancer-specific promoter is the PEG-3 promoter.

22. The nucleic acid construct of claim 20, wherein the one or more therapeutic genes is a cytokine, a thymidine kinase, a toxin, a pathogen-associated molecular pattern (PAMP), a danger-associated molecular pattern (DAMP), an immune checkpoint inhibitor gene, or any combination thereof.

23. The nucleic acid construct of claim 22, wherein the thymidine kinase is HSV1-TK.

24. The nucleic acid construct of claim 22, wherein the PAMP is flagellin (FliC).

25. The nucleic acid construct of claim 20, wherein if multiple therapeutic genes are present, the multiple therapeutic genes are separated by a picornavirus 2A ribosome skipping sequence.

26. The nucleic acid construct of claim 25, wherein the picornavirus ribosome skipping sequence is P2A or T2A.

27. The nucleic acid construct of claim 20, wherein the therapeutic gene is engineered to have a reduced CpG content compared to its wild-type counterpart.

28. The nucleic acid construct of claim 20, wherein the nucleic acid construct comprises a CpG-free plasmid backbone.

29. The nucleic acid construct of claim 20, wherein the nucleic acid construct is formulated into nanoparticles with a cationic polymer.

30. The nucleic acid construct of claim 29, wherein the nanoparticles are prepared at a N/P ratio of 4 or 6.

31. The nucleic acid construct of claim 29, wherein the nanoparticles are lyophilized.

32. The nucleic acid construct of claim 20, wherein the nucleic acid construct is delivered systemically.

33. The nucleic acid construct of claim 20, wherein the cancer is selected from the group consisting of breast cancer, melanoma, carcinoma of unknown primary (CUP), neuroblastoma, malignant glioma, cervical cancer, colon cancer, hepatocarcinoma, ovarian cancer, lung cancer, pancreatic cancer, and prostate cancer.

34. The nucleic acid construct of claim 22, wherein the immune checkpoint inhibitor gene encodes a monoclonal antibody selected from the group consisting of an anti-PD-1 antibody, an anti-PD-L1 antibody, and an anti-CTLA-4 antibody.

35. The nucleic acid construct of claim 22, wherein the immune checkpoint inhibitor gene encodes an immune checkpoint inhibitor fusion protein comprising a PD-1 fusion protein.

36. The nucleic acid construct of claim 35, wherein the PD-1 fusion protein comprises a fusion of PD-1 and an immunoglobulin Fc region.

37. The nucleic acid construct of claim 22, wherein the cytokine is selected from the group consisting of IL-12, IL-24, IL-2, IL-15, and GM-CSF.

38. The nucleic acid construct of claim 22, wherein the cytokine is a single chain variant of IL-12 (scIL-12).

39. A composition for the treatment of cancer comprising an expression cassette, wherein the expression cassette comprises a cancer-specific promoter and one or more therapeutic genes.

40. The composition of claim 39, wherein the cancer-specific promoter is the PEG-3 promoter.

41. The composition of claim 39, wherein the one or more therapeutic genes is a cytokine, a thymidine kinase, a toxin, a pathogen-associated molecular pattern (PAMP), a danger-associated molecular pattern (DAMP), an immune checkpoint inhibitor gene, or any combination thereof.

42. The composition of claim 41, wherein the thymidine kinase is HSV1-TK.

43. The composition of claim 41, wherein the PAMP is flagellin (FliC).

44. The composition of claim 39, wherein if multiple therapeutic genes are present, the multiple therapeutic genes are separated by a picornavirus 2A ribosome skipping sequence.

45. The composition of claim 44, wherein the picornavirus ribosome skipping sequence is P2A or T2A.

46. The composition of claim 39, wherein the therapeutic gene is engineered to have a reduced CpG content compared to its wild-type counterpart.

47. The composition of claim 39, wherein the nucleic acid construct comprises a CpG-free plasmid backbone.

48. The composition of claim 39, wherein the nucleic acid construct is formulated into nanoparticles with a cationic polymer.

49. The composition of claim 48, wherein the nanoparticles are prepared at a N/P ratio of 4 or 6.

50. The composition of claim 48, wherein the nanoparticles are lyophilized.

51. The composition of claim 39, wherein the nucleic acid construct is delivered systemically.

52. The composition of claim 39, wherein the cancer is selected from the group consisting of breast cancer, melanoma, carcinoma of unknown primary (CUP), neuroblastoma, malignant glioma, cervical cancer, colon cancer, hepatocarcinoma, ovarian cancer, lung cancer, pancreatic cancer, and prostate cancer.

53. The composition of claim 41, wherein the immune checkpoint inhibitor gene encodes a monoclonal antibody selected from the group consisting of an anti-PD-1 antibody, an anti-PD-L1 antibody, and an anti-CTLA-4 antibody.

54. The composition of claim 41, wherein the immune checkpoint inhibitor gene encodes an immune checkpoint inhibitor fusion protein comprising a PD-1 fusion protein.

55. The composition of claim 54, wherein the PD-1 fusion protein comprises a fusion of PD-1 and an immunoglobulin Fc region.

56. The composition of claim 41, wherein the cytokine is selected from the group consisting of IL-12, IL-24, IL-2, IL-15, and GM-CSF.

57. The composition of claim 41, wherein the cytokine is a single chain variant of IL-12 (scIL-12).

58. The nanoparticles of any one of claim 11, 29, or 48, wherein the cationic polymer is linear polyethylenimine.


SEQUENCE LISTING (25 sequences)

SEQ ID NO: 1 - DNA, 1148 bp, Artificial Sequence
  Description of Artificial Sequence Synthetic polynucleotide
ccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaatggatcctgataagctagc

SEQ ID NO: 2 - DNA, 1148 bp, Artificial Sequence
  Description of Artificial Sequence Synthetic polynucleotide
ccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccattttcctggacagacatcccattgccttcatgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaatggatcctgataagctagc

SEQ ID NO: 3 - DNA, 509 bp, Artificial Sequence
  Description of Artificial Sequence Synthetic polynucleotide
aagcttggcattccggtactgttggtaaagccaccatgtacaggatgcaactcctgtcttgcattgcactgagtcttgcacttgtcacaaacagtgcacctacttcaagttctacaaagaaaacacagctgcaactggagcatctcctgctggatctgcagatgatcttgaatggaattaataattacaagaatcccaaactcaccaggatgctcacatttaagttttacatgcccaagaaggccacagaactgaaacatcttcagtgtctggaagaagaactcaaacctctggaggaagtgctcaatctggctcaaagcaaaaactttcacctgagacccagggacctgatcagcaatatcaatgtaattgttctggaactcaagggatctgaaacaacattcatgtgtgaatatgctgatgagacagcaaccattgtggaatttctgaacagatggattaccttttgtcaaagcatcatctcaacactgacttaaacctgagctagc

SEQ ID NO: 4 - DNA, 557 bp, Artificial Sequence
  Description of Artificial Sequence Synthetic polynucleotide
aagcttggcattccggtactgttggtaaagccaccatgtacagcatgcagctggcctcctgtgtgacactgacactggtgctgctggtgaactctgcacccacttcaagctccacctcaagctctacagctgaagcccagcagcagcagcagcagcagcagcagcagcagcagcacctggagcagctgctgatggacctgcaggagctgctgagcaggatggagaattacaggaacctgaaactccccaggatgctgaccttcaaattttacttgcccaagcaggccacagaactgaaggatctgcagtgcctggaagatgaacttggacctctgaggcatgtgctggatctgactcaaagcaagagctttcaactggaagatgctgagaatttcatcagcaatatcagagtgactgtggtcaaactgaagggctctgacaacacatttgagtgccaatttgatgatgagtcagccactgtggtggactttctgaggagatggattgccttctgtcaaagcatcatctcaacaagccctcaataaacctgagctagc

SEQ ID NO: 5 - DNA, 1698 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttcctaaacctgagctagc

SEQ ID NO: 6 - DNA, 1679 bp, Artificial Sequence
  Description of Artificial Sequence Synthetic polynucleotide
aagcttggcattccggtactgttggtaaagccaccatgtgtcctcagaagctcaccatctcctggtttgccattgttttgctggtgtctccactcatggccatgtgggagctggagaaagatgtttatgttgtggaggtggactggactcctgatgcccctggagaaacagtgaacctcacctgtgacacccctgaagaagatgacatcacctggacctcagaccagagacatggagtcataggctctggaaagaccctgaccatcactgtcaaagagtttctggatgctggccagtacacctgccacaaaggaggggagactctgagccactcacatctgctgctccacaagaaggaaaatggaatttggtccactgaaattctgaaaaatttcaaaaacaagactttcctgaagtgtgaagcaccaaattactctggaaggttcacctgctcatggctggtgcaaagaaacatggacttgaagttcaacatcaagagcagtagcagttcccctgactctagggcagtgacatgtggaatggcctctctgtctgcagagaaggtcacactggaccaaagggactatgagaagtattcagtgtcctgccaggaggatgtcacctgcccaactgctgaggagaccctgcccattgaactggccttggaagcaaggcagcagaataaatatgagaactacagcaccagcttcttcatcagggacatcatcaaaccagaccctcccaagaacttgcagatgaagcctttgaagaactcacaggtggaggtcagctgggagtaccctgactcctggagcactccccattcctacttctccctcaagttctttgttagaatccagaggaagaaagaaaagatgaaggagacagaggaggggtgtaaccagaaaggtgccttcctggtggagaagacatctacagaagtccaatgcaaaggagggaatgtctgtgtgcaagctcaggataggtattacaattcctcatgcagcaagtgggcatgtgttccctgcagggtcagatctggtggaggtggaagtggaggtggtggatctgggggtggaggcagcagggtcattccagtctctggacctgccaggtgtcttagccagtccagaaacctgctgaagaccacagatgacatggtgaagactgccagagaaaaactgaaacattattcctgcactgctgaagacattgatcatgaagacatcacaagggaccaaaccagcacattgaagacctgtctgccactggaactgcacaagaatgagagttgcctggctactagagagacttcttccacaacaagagggagctgcctgcccccacagaagacctctttgatgatgaccctgtgccttggtagcatctatgaggacttgaagatgtaccagacagagttccaggccatcaatgcagcacttcagaatcacaaccatcagcagatcattctggacaagggcatgctggtggccattgatgagctgatgcagtctctgaatcataatggagagactctgagacagaaacctcctgtgggagaagcagacccttacagagtgaaaatgaagctctgcatcctgcttcatgccttcagcaccagagtggtgaccatcaacagggtgatgggctatctgagctctgcctaaacctgagctagc

SEQ ID NO: 7 - DNA, 438 bp, Artificial Sequence
  Description of Artificial Sequence Synthetic polynucleotide
cgtctcagacctatgtacaggatgcaactcctgtcttgcattgcactgagtcttgcacttgtcacaaacagtgcaggagccaactgggtgaatgtgatcagtgatttgaaaaaaattgaagatcttattcaatctatgcatattgatgctactttgtatacagaaagtgatgttcaccccagttgcaaagtgacagcaatgaagtgctttctcttggagctgcaagttatttcacttgagtctggagatgcaagtattcatgatacagtggaaaatctgatcatcctggcaaacaacagtttgtcttctaatgggaatgtgacagaatctggatgcaaagaatgtgaggaactggaggaaaaaaatattaaagaatttttgcagagttttgtgcatattgtccaaatgttcatcaacacttcttaaacctgagctagc

SEQ ID NO: 8 - DNA, 668 bp, Artificial Sequence
  Description of Artificial Sequence Synthetic polynucleotide
aagcttggcattccggtactgttggtaaagccaccatgaattttcaacagaggctgcaaagcctgtggactctggccagacccttctgccctcctttgctggccacagcctctcaaatgcagatggttgtgctcccttgcctgggttttaccctgcttctctggagccaggtgtcaggggcccagggccaagaattccactttgggccctgccaagtgaagggggttgttccccagaaactgtgggaagccttctgggctgtgaaagacactatgcaagctcaggataacatcaccagtgccaggctgctgcagcaggaggttctgcagaatgtctctgatgctgagagctgttaccttgtccacaccctgctggagttctacttgaaaactgttttcaaaaactaccacaatagaacagttgaagtcaggactctgaagtcattctctactctggccaacaactttgttctcattgtgtcacaactgcaacccagtcaagaaaatgagatgttttccatcagagacagtgcacacaggaggtttctgctgttcagaagagcattcaaacagttggatgtggaagcagctctgaccaaagcccttggggaagtggacattcttctgacctggatgcagaaattctacaagctctaaacctgagctagc

SEQ ID NO: 9 - DNA, 499 bp, Artificial Sequence
  Description of Artificial Sequence Synthetic polynucleotide
ggatcctctggaagtggagctactaacttcagcctgctgaagcaggctggagatgtggctgcagagcctgctgctcttgggcactgtggcctgcagcatctctgcacctgccagaagccccagccccagcacccagccctgggagcatgtgaatgccatccaggaggccaggaggctcctgaacctgagtagagacactgctgctgagatgaatgaaacagtggaagtcatctcagaaatgtttgacctccaggagcccacctgcctccagaccaggctggagctgtacaagcagggcctgaggggcagcctcaccaagctcaagggccccttgaccatgatggccagccactacaagcagcactgccctccaacccctgaaacttcctgtgcaacccagattatcacctttgaaagtttcaaagagaacctgaaggactttctgcttgtcatcccctttgactgctgggagccagtccaggagtgaacctgagctagc

SEQ ID NO: 10 - DNA, 513 bp, Artificial Sequence
  Description of Artificial Sequence Synthetic polynucleotide
ggatcctctggaagtggagctactaacttcagcctgctgaagcaggctggagatgtggaggagaaccctggacctatgtggctgcagaatctgcttttcctgggcattgtggtctacagcctctcagcacccaccaggtcacccatcactgtcaccagaccttggaagcatgtagaggccatcaaagaagccctgaacctcctggatgacatgcctgtcaccttgaatgaagaggtagaagtggtctctaatgagttctccttcaagaagctgacatgtgtgcagaccagactgaagatatttgagcagggtctaaggggcaatttcaccaaactcaagggagccttgaacatgacagccagctactaccagacatactgccccccaactcctgaaacagactgtgaaacacaagttaccacctatgctgatttcatagacagccttaaaacctttctgactgatatcccctttgaatgcaaaaaaccaggccaaaaatgaacctgagctagc

SEQ ID NO: 11 - DNA, 1339 bp, Artificial Sequence
  Description of Artificial Sequence Synthetic polynucleotide
ggatcctctggaagtggagctactaacttcagcctgctgaagcaggctggagatgtggaggagaaccctggacctatggactggacctggagggtcttctgtttgctggctgtaactccaggtgcccaccccctggactccccagacaggccctggaacccccccaccttctccccagccctgctggtggtgactgaaggggacaatgccaccttcacctgcagcttctccaacacatctgagagctttgtgctgaactggtacaggatgagccccagcaaccagactgacaagctggctgccttccctgaggacaggagccagcctggccaggactgcagattcagggtcacacaactgcccaatgggagggacttccacatgagtgtggtcagggccaggagaaatgacagtggcacctacctctgtggggccatctccctggcccccaaggcccagatcaaagagagcctgagggcagagctcagggtgacagagagaagggcagaagtgcccacagcccaccccagcccctcacccaggtcagctggccagttccaaaccctggtggagtccaaatatggtcccccatgcccaccatgcccagcacctgagtttgaggggggaccatcagtcttcctgttccccccaaaacccaaggacactctcatgatctccaggacccctgaggtcacctgtgtggtggtggatgtgagccaggaagaccctgaggtccagttcaactggtatgtggatggggtggaggtgcataatgccaagacaaagcctagggaggagcagttcaacagcacctacagagtggtcagtgtcctcacagtcctgcaccaggactggctgaatggcaaggagtacaagtgcaaggtctccaacaaaggcctcccctcctccattgagaaaaccatctccaaagccaaagggcagcccagagagccacaggtgtacaccctgcccccatcccaggaggagatgaccaagaaccaggtcagcctgacctgcctggtcaaaggcttctaccccagtgacattgctgtggagtgggagagcaatgggcagcctgagaacaactacaagaccacacctccagtgctggactctgatggctccttcttcctctacagcaggctcacagtggacaagagcaggtggcaggaggggaatgtcttctcatgctctgtgatgcatgaggctctgcacaaccactacacacagaagagcctctccctgtctctgggtaaaagaaggaagaggggaagtggagagggcagaggaagtctgctaacatgtggtgatgtggaggagaatcctgggacctgagacg

SEQ ID NO: 12 - DNA, 1143 bp, Artificial Sequence
  Description of Artificial Sequence Synthetic polynucleotide
cgtctcagacctatggagacagacacactcctgctgtgggtgctgctgctctgggttccaggttccactggtgacatggcacaagtcattaatacaaacagcctgtctctgttgacccagaataacctgaacaaatcccagtcagctctgggcacagctattgagagactgtcttctggtctgaggatcaacagtgccaaagatgatgctgcaggtcaggccattgctaacaggtttactgccaacatcaaaggtctgactcaggcttccagaaatgctaatgatggtatctccattgcccagaccactgaaggagctctgaatgaaatcaacaacaacctgcagagagtgagggaactggctgttcagtctgctaacagcaccaactcccagtctgacctggactccatccaggctgaaatcacccagagactgaatgaaattgacagagtgtctggccagactcagttcaatggagtgaaagtcctggcccaggacaacaccctgaccatccaggttggtgccaatgatggtgaaactattgatattgatctgaagcagatcaactctcagaccctgggtctggataccctgaatgtgcaacaaaaatataaggtcagtgatacagctgcaactgttacaggatatactcaaaataaagatggttccatcagtattaatactacaaaatacactgcagatgatggtacatccaaaactgcactgaacaaactgggtggggcagatggcaaaacagaagttgtttctattggtggtaaaacttatgctgcaagtaaagctgaaggtcacaactttaaagcacagcctgatctggctgaagctgctgctacaaccacagaaaaccctctgcagaaaattgatgctgctttggcacaggttgacaccctgagatctgacctgggtgctgtgcagaacaggttcaactctgctattaccaacctgggcaacacagtgaacaacctgacttctgccagaagcaggattgaagattctgactatgccacagaagtttccaacatgtctagagcccagattctgcagcaggctggtacctctgttctggcccaggccaaccaggttccccaaaatgtcctctctctgctgagataaacctgagctagc

SEQ ID NO: 13 - DNA, 3972 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttccaggagaaagagaggatcctctggaagtggagctactaacttcagcctgctgaagcaggctggagatgtggaggagaaccctggacctatggagtttgggctgagctgggttttccttgttgctcttttaagaggtgtccagtgtcaggtgcagttggtggagtctggggggggtgtggtgcagccagggaggtcactgagactgagttgtgcagcaagtgggtttacatttagtagttatacaatgcattgggttaggcaagctccagggaagggtctggagtgggtgacttttatttcttatgatggtaataataaatattatgcagattcagttaagggaaggtttactattagtagggataattcaaaaaatactctgtatttgcagatgaattctctgagggctgaggatacagctatttattattgtgctagaactggttggctgggtccatttgattattgggggcagggaacacttgtgacagtgtcatcagcttcaacaaaaggtccatctgtttttccattggctccttcttctaagtcaacttctggtggaactgcagctctgggatgtctggtgaaggattattttccagaacctgtgactgtttcttggaatagtggtgctctgactagtggagttcatacttttccagctgttctgcagagttctggactgtattctctgagtagtgtggttacagttccatcaagttctctgggtactcaaacttatatttgtaatgtgaatcataagccttcaaatacaaaggtggataaaagggtggagccaaagtcatgtgataagactcatacatgtcctccatgtcctgctccagagcttctgggggggccatctgtttttctgtttccaccaaagcctaaggatactcttatgattagtaggacaccagaagttacatgtgtggtggttgatgtgtctcatgaagatccagaggtgaagtttaattggtatgttgatggggtggaggttcataatgcaaagacaaagcctagggaggaacagtataatagtacatatagagtggtgtctgtgctgactgtgctgcatcaggattggctgaatggaaaagagtataagtgtaaagtgtcaaataaggctctgcctgcacctattgaaaaaacaatttcaaaggcaaaagggcagccaagggagcctcaagtttatactctgccaccttcaagggatgaacttacaaagaatcaagtgagtttgacttgtcttgtgaaaggattttatccttcagatattgctgtggagtgggagtcaaatggtcagcctgaaaataattataagactactccaccagtgctggatagtgatgggtctttttttctgtatagtaagctgactgtggataagtctaggtggcagcagggaaatgtgttttcttgtagtgtgatgcatgaggctctgcataatcattatacacagaagtctctgagtttgtctcctggtaaaagaaggaagaggggaagtggagagggcagaggaagtctgctaacatgtggtgatgtggaggagaatcctggacctatggacatgagggtccctgctcagctcctggggctcctgctgctctggctctcaggtgccagatgtgagattgtgctgacacaatctccaggaactttgagtctgtctccaggtgagagggctacactgtcatgtagggcatcacagtctgttggaagttcttatctggcttggtatcaacaaaagcctgggcaggctccaagactgctgatttatggtgctttttctagagctactggaattcctgataggtttagtgggagtgggagtggaacagattttacactgactatttctagactggaaccagaagattttgcagtgtattattgtcagcagtatgggtcttcaccttggacttttggtcagggaactaaagtggaaattaagagaactgttgctgctccttcagtttttatttttccacctagtgatgagcagctgaagagtggaacagcatctgtggtgtgtcttttgaataatttttatcctagagaagctaaggtgcagtggaaagtggataatgcattgcagagtggaaattcacaagaatcagtgactgagcaggattcaaaagatagtacatatagtctttcatctactttgacactgtctaaggctgattatgagaagcataaagtgtatgcatgtgaggtgacacatcaggggctgtcttcacctgtgacaaagtcttttaatagaggggagtgttgaacctgagctagc

SEQ ID NO: 14 - DNA, 3978 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttccaggagaaagagaggatcctctggaagtggagctactaacttcagcctgctgaagcaggctggagatgtggaggagaaccctggacctatggagtttgggctgagctgggttttccttgttgctcttttaagaggtgtccagtgtcaggtgcagttggtgcagtctggagttgaagtgaaaaagcctggtgcttcagtgaaggtgagttgtaaggcttcagggtatacatttactaattattatatgtattgggtgagacaggctcctggtcagggacttgagtggatgggtggaattaatccttctaatggtggaactaattttaatgagaagtttaagaatagagtgactctgactacagatagttctactactactgcttatatggagctgaagtctctgcagtttgatgatacagctgtgtattattgtgctagaagagattatagatttgatatgggatttgattattggggtcaggggacaacagttacagttagttcagcttctactaaaggaccatcagtttttcctctggcaccatgttctaggagtacatcagagtctactgctgcacttgggtgtttggtgaaagattattttccagaacctgttacagtgagttggaatagtggagctctgacatcaggggttcatacttttcctgctgtgttgcagtcatctgggctgtattctctgtcatctgttgtgacagtgccaagtagttcattgggaactaaaacttatacatgtaatgtggatcataagccttctaatactaaagtggataagagggtggaatctaagtatggaccaccatgtcctccatgtccagcacctgaatttctgggaggaccatctgtgtttttgtttccaccaaaaccaaaagatacattgatgatttcaaggacaccagaggtgacatgtgtggtggtggatgtgagtcaggaagatcctgaagtgcaatttaattggtatgtggatggagtggaggttcataatgctaaaactaagcctagggaagagcagtttaatagtacatatagggtggtgtctgtgcttacagttctgcatcaagattggctgaatggaaaagagtataagtgtaaagttagtaataaagggctgccttcttcaattgagaaaacaattagtaaggcaaagggtcagcctagagagcctcaagtttatacattgccaccttctcaggaagagatgacaaagaatcaggtgtctctgacatgtttggttaagggtttttatccatcagatattgctgtggagtgggagtcaaatggtcaaccagagaataattataaaactacaccaccagtgctggattcagatgggtcattttttctgtatagtagactgactgtggataaatcaaggtggcaggagggaaatgtgttttcttgttctgtgatgcatgaagctctgcataatcattatacacagaaatcattgagtctgtcattgggtaagagaaggaagaggggaagtggagagggcagaggaagtctgctaacatgtggtgatgtggaggagaatcctggacctatggacatgagggtccctgctcagctcctggggctcctgctgctctggctctcaggtgccagatgtgaaattgtgctgactcagagtcctgctacactgtcattgagtccaggggaaagagcaacactgtcttgtagggcaagtaagggagtttcaacttctggttattcatatctgcattggtatcagcagaaaccagggcaggcacctaggttgctgatttatctggcatcatatttggagagtggggttcctgcaagattttctggatctgggtcaggaacagattttacactgacaatttcaagtcttgagcctgaggattttgcagtttattattgtcagcattcaagagatctgcctctgacttttggaggaggtacaaaggttgagattaaaagaactgtggcagcaccttcagtgtttatttttcctcctagtgatgagcaattgaaaagtggtacagcatctgttgtgtgtctgcttaataatttttatcctagagaggcaaaagttcagtggaaggttgataatgcattgcaatctgggaattctcaagagagtgttacagaacaggattcaaaagattctacttattcactgtcatcaactctgacactgtcaaaggcagattatgagaagcataaagtgtatgcttgtgaggtgactcatcaagggcttagttctcctgttactaaaagttttaatagaggtgagtgttgaacctgagctagc

SEQ ID NO: 15 - DNA, 3945 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttccaggagaaagagaggatcctctggaagtggagctactaacttcagcctgctgaagcaggctggagatgtggaggagaaccctggacctatggagtttgggctgagctgggttttccttgttgctcttttaagaggtgtccagtgtcaggtgcagctggtggagagtggtgggggggtggtgcaacctgggaggagtctgagactggattgtaaggctagtgggattactttttcaaatagtggaatgcattgggtgagacaggctcctgggaaggggcttgagtgggttgctgtgatttggtatgatgggtctaaaaggtattatgctgatagtgtgaagggtagatttacaatttctagggataatagtaaaaatactctgtttcttcagatgaattctttgagagcagaggatacagcagtttattattgtgcaacaaatgatgattattgggggcagggtactctggttactgtgtcttctgcttctacaaaggggccatcagtgtttcctctggcaccttgtagtagatcaactagtgagagtacagctgctctggggtgtcttgtgaaagattattttcctgaacctgtgactgtgtcttggaattctggagcacttacttcaggtgttcatacatttccagcagtgctgcagagttctgggctgtatagtctgtcttcagtggtgacagtgccttcatcaagtctgggaacaaaaacttatacatgtaatgtggatcataagccatcaaatactaaggtggataagagagtggaatctaagtatggtccaccatgtcctccttgtccagctcctgaatttctggggggacctagtgtgtttttgtttccacctaagcctaaggatacacttatgatttcaagaactcctgaggttacttgtgtggtggtggatgtgtctcaggaagatccagaagtgcaatttaattggtatgtggatggggttgaagtgcataatgcaaaaacaaaaccaagggaggagcagtttaattctacttatagggtggtgtctgtgcttacagtgctgcatcaagattggttgaatgggaaagaatataagtgtaaggtttctaataaggggttgccttctagtattgagaagactatttctaaggcaaaggggcagcctagagaacctcaagtttatacacttcctccaagtcaggaggagatgactaaaaatcaggtttcactgacatgtctggtgaaaggattttatccatcagatattgcagttgagtgggaatctaatgggcagcctgagaataattataagactacaccacctgtgcttgattctgatggaagtttttttctgtatagtagactgacagtggataaaagtagatggcaggaaggtaatgtgttttcttgttctgtgatgcatgaggcactgcataatcattatactcaaaagagtctgtctctgtctcttggaaagagaaggaagaggggaagtggagagggcagaggaagtctgctaacatgtggtgatgtggaggagaatcctggacctatggacatgagggtccctgctcagctcctggggctcctgctgctctggctctcaggtgccagatgtgagattgtgctgacacagtctcctgcaactctgtctctgtcacctggggagagggctactctgtcatgtagggctagtcagtctgtgtcatcatatctggcatggtatcagcaaaaaccaggtcaagctccaaggctgctgatttatgatgcatcaaatagggcaactggtattccagcaaggttttctgggtcaggaagtggaacagattttacactgactattagttctctggagccagaggattttgcagtgtattattgtcaacagagttctaattggccaagaacatttgggcagggtacaaaagtggagattaaaaggacagtggctgctccttctgtgtttatttttccaccttcagatgaacaacttaaaagtggtacagcatcagtggtgtgtctgttgaataatttttatccaagggaagctaaagttcagtggaaagttgataatgcactgcagtctgggaattctcaggaatctgttacagaacaggattcaaaagattcaacttattctctttctagtactctgacattgtctaaggctgattatgaaaagcataaggtgtatgcttgtgaggtgacacatcagggacttagttcaccagtgactaaatcttttaataggggagagtgttgaacctgagctagc

SEQ ID NO: 16 - DNA, 3984 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttccaggagaaagagaggatcctctggaagtggagctactaacttcagcctgctgaagcaggctggagatgtggaggagaaccctggacctatggagtttgggctgagctgggttttccttgttgctcttttaagaggtgtccagtgtgaagtgcagcttgtggagtcaggaggggggctggtgcagcctggggggagtctgaggctgagttgtgcagcaagtggttatacttttacaaattatggaatgaattgggtgagacaggctcctggtaaagggctggagtgggttgggtggattaatacttatacaggggagccaacatatgctgcagattttaaaaggaggtttacttttagtctggatacatctaagtcaacagcttatcttcagatgaattctcttagggctgaggatacagctgtttattattgtgcaaagtatcctcattattatggatcatctcattggtattttgatgtgtggggtcagggaacactggtgactgttagtagtgctagtactaaagggccttcagtgtttccacttgctccatcaagtaagtcaacatctggagggactgctgcactggggtgtttggtgaaggattattttccagaaccagtgactgtttcttggaattctggagcacttacttctggtgtgcatacatttcctgcagtgttgcagtcatcaggattgtattcactgtcttctgtggtgactgtgccatcaagttcactgggaacacagacatatatttgtaatgttaatcataaaccttctaatacaaaggtggataagaaggtggaacctaaatcttgtgataaaacacatacttgtccaccttgtccagctccagaactgcttgggggtccatctgtgtttctttttcctcctaagcctaaagatacacttatgatttctagaacaccagaagttacttgtgtggtggtggatgtgagtcatgaggacccagaagttaagtttaattggtatgtggatggggttgaagtgcataatgctaaaacaaagcctagagaagaacagtataatagtacatatagagtggtgtctgtgctgactgtgctgcatcaggattggctgaatggaaaggaatataaatgtaaggtgagtaataaagctcttccagctcctattgagaagacaatttctaaggctaaggggcaaccaagggaaccacaagtgtatacattgccaccttcaagggaggagatgactaagaatcaggtgtctctgacttgtcttgttaaagggttttatcctagtgatattgctgtggagtgggagtcaaatggacagccagaaaataattataaaacaacaccacctgtgctggatagtgatggaagtttttttctgtattctaagctgacagtggataagagtagatggcagcagggtaatgtgtttagttgtagtgttatgcatgaagcactgcataatcattatacacagaaatctctttctctgtcaccagggaaaagaaggaagaggggaagtggagagggcagaggaagtctgctaacatgtggtgatgtggaggagaatcctggacctatggacatgagggtccctgctcagctcctggggctcctgctgctctggctctcaggtgccagatgtgatattcagatgacacagtcaccaagttctcttagtgcttctgtgggggatagagttacaattacttgttcagcaagtcaggatattagtaattatcttaattggtatcagcagaagcctggaaaggctcctaaggtgttgatttattttactagttcactgcattctggtgttcctagtaggtttagtgggtctggatcaggaacagattttacactgacaatttcatcactgcagcctgaagattttgctacttattattgtcagcagtatagtactgttccttggacatttgggcagggtacaaaggtggagattaaaagaactgtggctgcacctagtgtttttatttttcctccttcagatgagcagctgaaatctggtacagcatctgttgtttgtctgcttaataatttttatcctagggaggcaaaggtgcaatggaaggtggataatgcactgcagagtggaaattctcaagaatcagtgactgagcaagattctaaagattcaacttattctctgagttcaactcttactctgtctaaggctgattatgaaaaacataaggtttatgcttgtgaggtgactcatcaaggacttagtagtcctgtgacaaagagttttaataggggggagtgttgaacctgagctagc

SEQ ID NO: 17 - DNA, 3363 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttccaggagaaagagaggatcctctggaagtggagctactaacttcagcctgctgaagcaggctggagatgtggaggagaaccctggacctatggacatgagggtccctgctcagctcctggggctcctgctgctctggctctcaggtgccagatgtgatattcagctgacacagagtcctgcaagtctggctgttagtctggggcaaagagcaacaattagttgtaaggcttctcagtcagtggattatgatggagatagttatctgaattggtatcagcagattcctgggcagcctcctaagcttctgatttatgatgcatcaaatcttgtgtcaggaattccaccaaggttttctggatctggaagtggaactgattttactctgaatattcatcctgtggaaaaagtggatgctgcaacatatcattgtcagcagtcaactgaggacccttggacatttggaggggggacaaagcttgagattaagggggggggaggatcaggagggggaggttctggagggggaggatctcaggtgcagctgcagcagtctggggctgagcttgttagaccaggatcttctgtgaaaatttcatgtaaagcatcagggtatgcttttagttcttattggatgaattgggtgaaacagaggcctggtcagggactggagtggattggacagatttggcctggggatggtgatactaattataatggaaagtttaaaggaaaagctacactgacagcagatgagtcttcatctactgcatatatgcagcttagttctctggcaagtgaggattcagcagtgtatttttgtgcaagaagggagactacaacagtgggaagatattattatgctatggattattggggacaaggaacaactgtgacagtgtcttctggggggggtgggtctgatattaaacttcagcaatcaggagcagagcttgcaaggccaggtgcttcagtgaaaatgtcatgtaagactagtgggtatacatttactaggtatactatgcattgggtgaaacaaagaccaggacaggggcttgagtggattggatatattaatccaagtaggggatatacaaattataatcaaaagtttaaagataaggctactctgactactgataagtcaagttctactgcttatatgcagctttcttctttgacttcagaggattcagcagtgtattattgtgcaagatattatgatgatcattattgtctggattattggggacaaggaacaacactgactgtgtcttctgtggagggagggagtggaggatcaggtgggtcaggaggtagtggaggggtggatgatattcaactgacacagtctccagctattatgagtgcatcaccaggggagaaggtgacaatgacttgtagagcatcaagttctgtttcttatatgaattggtatcagcagaagtctgggacaagtcctaaaagatggatttatgatacttctaaagtggcatctggagtgccttataggtttagtggatctggatctggaacatcttattcattgactattagtagtatggaagcagaagatgcagcaacttattattgtcagcagtggtcatcaaatcctcttacatttggagctgggactaagttggaattgaaacatcatcatcatcatcattgaacctgagctagc

SEQ ID NO: 18 - DNA, 3321 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttccaggagaaagagaggatcctctggaagtggagctactaacttcagcctgctgaagcaggctggagatgtggaggagaaccctggacctatggagtttgggctgagctgggttttccttgttgctcttttaagaggtgtccagtgtgaagtgcagcttgtggagtcaggaggggggctggtgcagcctggggggagtctgaggctgagttgtgcagcaagtggttatacttttacaaattatggaatgaattgggtgagacaggctcctggtaaagggctggagtgggttgggtggattaatacttatacaggggagccaacatatgctgcagattttaaaaggaggtttacttttagtctggatacatctaagtcaacagcttatcttcagatgaattctcttagggctgaggatacagctgtttattattgtgcaaagtatcctcattattatggatcatctcattggtattttgatgtgtggggtcagggaacactggtgactgttagtagtgctagtactaaagggccttcagtgtttccacttgctccatcaagtaagtcaacatctggagggactgctgcactggggtgtttggtgaaggattattttccagaaccagtgactgtttcttggaattctggagcacttacttctggtgtgcatacatttcctgcagtgttgcagtcatcaggattgtattcactgtcttctgtggtgactgtgccatcaagttcactgggaacacagacatatatttgtaatgttaatcataaaccttctaatacaaaggtggataagaaggtggaacctaaatcttgtgataaaacacatactctgagaaggaagaggggaagtggagagggcagaggaagtctgctaacatgtggtgatgtggaggagaatcctggacctatggacatgagggtccctgctcagctcctggggctcctgctgctctggctctcaggtgccagatgtgatattcagctgacacagtcaccaagttctcttagtgcttctgtgggggatagagttacaattacttgttcagcaagtcaggatattagtaattatcttaattggtatcagcagaagcctggaaaggctcctaaggtgttgatttattttactagttcactgcattctggtgttcctagtaggtttagtgggtctggatcaggaacagattttacactgacaatttcatcactgcagcctgaagattttgctacttattattgtcagcagtatagtactgttccttggacatttgggcagggtacaaaggtggagattaaaagaactgtggctgcacctagtgtttttatttttcctccttcagatgagcagctgaaatctggtacagcatctgttgtttgtctgcttaataatttttatcctagggaggcaaaggtgcaatggaaggtggataatgcactgcagagtggaaattctcaagaatcagtgactgagcaagattctaaagattcaacttattctctgagttcaactcttactctgtctaaggctgattatgaaaaacataaggtttatgcttgtgaggtgactcatcaaggacttagtagtcctgtgacaaagagttttaataggggggagtgttgaacctgagctagc

SEQ ID NO: 19 - DNA, 3953 bp, Artificial Sequence
  Description of Artificial Sequence Synthetic polynucleotide
aagcttggcattccggtactgttggtaaagccaccatgtgtcctcagaagctcaccatctcctggtttgccattgttttgctggtgtctccactcatggccatgtgggagctggagaaagatgtttatgttgtggaggtggactggactcctgatgcccctggagaaacagtgaacctcacctgtgacacccctgaagaagatgacatcacctggacctcagaccagagacatggagtcataggctctggaaagaccctgaccatcactgtcaaagagtttctggatgctggccagtacacctgccacaaaggaggggagactctgagccactcacatctgctgctccacaagaaggaaaatggaatttggtccactgaaattctgaaaaatttcaaaaacaagactttcctgaagtgtgaagcaccaaattactctggaaggttcacctgctcatggctggtgcaaagaaacatggacttgaagttcaacatcaagagcagtagcagttcccctgactctagggcagtgacatgtggaatggcctctctgtctgcagagaaggtcacactggaccaaagggactatgagaagtattcagtgtcctgccaggaggatgtcacctgcccaactgctgaggagaccctgcccattgaactggccttggaagcaaggcagcagaataaatatgagaactacagcaccagcttcttcatcagggacatcatcaaaccagaccctcccaagaacttgcagatgaagcctttgaagaactcacaggtggaggtcagctgggagtaccctgactcctggagcactccccattcctacttctccctcaagttctttgttagaatccagaggaagaaagaaaagatgaaggagacagaggaggggtgtaaccagaaaggtgccttcctggtggagaagacatctacagaagtccaatgcaaaggagggaatgtctgtgtgcaagctcaggataggtattacaattcctcatgcagcaagtgggcatgtgttccctgcagggtcagatctggtggaggtggaagtggaggtggtggatctgggggtggaggcagcagggtcattccagtctctggacctgccaggtgtcttagccagtccagaaacctgctgaagaccacagatgacatggtgaagactgccagagaaaaactgaaacattattcctgcactgctgaagacattgatcatgaagacatcacaagggaccaaaccagcacattgaagacctgtctgccactggaactgcacaagaatgagagttgcctggctactagagagacttcttccacaacaagagggagctgcctgcccccacagaagacctctttgatgatgaccctgtgccttggtagcatctatgaggacttgaagatgtaccagacagagttccaggccatcaatgcagcacttcagaatcacaaccatcagcagatcattctggacaagggcatgctggtggccattgatgagctgatgcagtctctgaatcataatggagagactctgagacagaaacctcctgtgggagaagcagacccttacagagtgaaaatgaagctctgcatcctgcttcatgccttcagcaccagagtggtgaccatcaacagggtgatgggctatctgagctctgccagaaggaagaggggatcctctggaagtggagctactaacttcagcctgctgaagcaggctggagatgtggaggagaaccctggacctatgatggtgttaagtcttctgtacctgttgacagcccttcctggtatcctgtcagaggtgcagctgcaggagtcaggaccaggcctggtgaaaccttctcagagtctgtccctgacttgttctgtcactgggtattcaattacatcttcatatagatggaactggatcaggaagtttccagggaataggctggagtggatggggtacataaattcagctggtatttctaattacaatccatctctgaagagaagaatctccatcacaagagacacatccaaaaaccagttctttctgcaggttaattctgtgactactgaggatgctgccacatattactgtgcaagaagtgataatatggggacaacaccttttacttattggggtcaagggacattggtgactgtgagttctgcatcaacaacagcaccatctgtctatccactggcccctgtgtgtggagatacaactggctcctcagtgactctgggatgcctggtcaagggttatttccctgagccagtgaccttgacctggaactctggctccctgtccagtggtgtgcacaccttcccagctgtcctgcagtctgacctctacaccctcagcagctcagtgactgtaacctctagcacctggcccagccagtccatcacctgcaatgtggcccacccagcaagcagcaccaaggtggacaagaaaattgagcccagagggcccacaatcaagccctgtcctccatgcaaatgcccagcacctaatgcagctggtggaccatctgtcttcatcttccctccaaagatcaaggatgtactcatgatctccctgagccccatagtcacatgtgtggtggtggatgtgagtgaggatgacccagatgtccagatcagctggtttgtgaacaatgtggaagtacacacagctcagacacaaacccatagagaggattacaacagtactctcagggtggtcagtgccctccccatccagcaccaggactggatgagtggcaaggagttcaaatgcaaggtcaacaacaaagacctgggggcacccattgagagaaccatctcaaaacccaaagggtcagtaagagctccacaggtatatgtcttgcctccaccagaagaagagatgactaagaaacaggtcactctgacctgcatggtcacagacttcatgcctgaagacatttatgtggagtggaccaacaatgggaaaacagagctgaactacaagaacactgaaccagtcctggactctgatggttcttacttcatgtacagcaagctgagagtggaaaagaagaactgggtggaaagaaatagctactcctgttcagtggtccatgagggtctgcacaatcaccacacaactaagagcttctctaggactccaagaaggaagaggggaagtggagagggcagaggaagtctgctaacatgtggtgatgtggaggagaatcctggacctatgaggtgcctagctgagttcctggggctgcttgtgctctggattcctggagccattggggatattgtgatgactcagggtactctgcctaatcctgtgccaagtggggagtctgtgtctattacatgtaggagttcaaagagtcttctttattcagatggaaaaacatatctgaattggtatctgcagagacctgggcagagtcctcagctgctgatttattggatgtctactagggcatctggggtgtctgatagattttctggtagtggtagtggtacagattttacattgaagatttctggggtggaggctgaagatgtgggtatttattattgtcagcaaggtctggagtttccaacatttgggggaggtactaagctggagctgaagagaactgatgctgcaccaactgtatccatcttcccaccatccagtgagcagctgacatctggaggtgcctcagttgtgtgcttcctgaacaacttctaccccaaagacatcaatgtcaagtggaagattgatggcagtgaaagacaaaatggggtcctgaacagttggactgatcaggacagcaaagacagcacctacagcatgagcagcaccctcaccctgaccaaggatgagtatgaaagacataacagctatacctgtgaggccactcacaagacatcaacttcacccattgtcaagagcttcaacaggaatgagtgttgaacctgagctagc

SEQ ID NO: 20 - DNA, 2261 bp, Artificial Sequence
  Description of Artificial Sequence Synthetic polynucleotide
aagcttggcattccggtactgttggtaaagccaccatgtacagcatgcagctggcctcctgtgtgacactgacactggtgctgctggtgaactctgcacccacttcaagctccacctcaagctctacagctgaagcccagcagcagcagcagcagcagcagcagcagcagcagcacctggagcagctgctgatggacctgcaggagctgctgagcaggatggagaattacaggaacctgaaactccccaggatgctgaccttcaaattttacttgcccaagcaggccacagaactgaaggatctgcagtgcctggaagatgaacttggacctctgaggcatgtgctggatctgactcaaagcaagagctttcaactggaagatgctgagaatttcatcagcaatatcagagtgactgtggtcaaactgaagggctctgacaacacatttgagtgccaatttgatgatgagtcagccactgtggtggactttctgaggagatggattgccttctgtcaaagcatcatctcaacaagccctcaaagaaggaagaggggaagtggagagggcagaggaagtctgctaacatgtggtgatgtggaggagaatcctggacctatgtgtcctcagaagctcaccatctcctggtttgccattgttttgctggtgtctccactcatggccatgtgggagctggagaaagatgtttatgttgtggaggtggactggactcctgatgcccctggagaaacagtgaacctcacctgtgacacccctgaagaagatgacatcacctggacctcagaccagagacatggagtcataggctctggaaagaccctgaccatcactgtcaaagagtttctggatgctggccagtacacctgccacaaaggaggggagactctgagccactcacatctgctgctccacaagaaggaaaatggaatttggtccactgaaattctgaaaaatttcaaaaacaagactttcctgaagtgtgaagcaccaaattactctggaaggttcacctgctcatggctggtgcaaagaaacatggacttgaagttcaacatcaagagcagtagcagttcccctgactctagggcagtgacatgtggaatggcctctctgtctgcagagaaggtcacactggaccaaagggactatgagaagtattcagtgtcctgccaggaggatgtcacctgcccaactgctgaggagaccctgcccattgaactggccttggaagcaaggcagcagaataaatatgagaactacagcaccagcttcttcatcagggacatcatcaaaccagaccctcccaagaacttgcagatgaagcctttgaagaactcacaggtggaggtcagctgggagtaccctgactcctggagcactccccattcctacttctccctcaagttctttgttagaatccagaggaagaaagaaaagatgaaggagacagaggaggggtgtaaccagaaaggtgccttcctggtggagaagacatctacagaagtccaatgcaaaggagggaatgtctgtgtgcaagctcaggataggtattacaattcctcatgcagcaagtgggcatgtgttccctgcagggtcagatctggtggaggtggaagtggaggtggtggatctgggggtggaggcagcagggtcattccagtctctggacctgccaggtgtcttagccagtccagaaacctgctgaagaccacagatgacatggtgaagactgccagagaaaaactgaaacattattcctgcactgctgaagacattgatcatgaagacatcacaagggaccaaaccagcacattgaagacctgtctgccactggaactgcacaagaatgagagttgcctggctactagagagacttcttccacaacaagagggagctgcctgcccccacagaagacctctttgatgatgaccctgtgccttggtagcatctatgaggacttgaagatgtaccagacagagttccaggccatcaatgcagcacttcagaatcacaaccatcagcagatcattctggacaagggcatgctggtggccattgatgagctgatgcagtctctgaatcataatggagagactctgagacagaaacctcctgtgggagaagcagacccttacagagtgaaaatgaagctctgcatcctgcttcatgccttcagcaccagagtggtgaccatcaacagggtgatgggctatctgagctctgcctaaacctgagctagc

SEQ ID NO: 21 - DNA, 3981 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttccaggagaaagagaggatcctctggaagtggagctactaacttcagcctgctgaagcaggctggagatgtggaggagaaccctggacctatggagtttgggctgagctgggttttccttgttgctcttttaagaggtgtccagtgtgaggtgcagctggtggaaagtggggggggacttgtgcagcctggggggtcacttaggctttcatgtgctgcttctgggtttacatttagtagatattggatgagttgggtgaggcaggcaccaggtaaggggctggagtgggtggctaatattaagcaagatggttctgagaagtattatgtggattctgttaagggtaggtttacaatttctagggataatgctaagaatagtctgtatctgcagatgaattcacttagagcagaggatactgcagtgtattattgtgctagagaagggggttggtttggtgaattggcatttgattattggggacaggggactctggttacagtgtcatcagcaagtactaaggggccatctgtttttcctctggctccttcatcaaagagtacaagtggaggtacagctgctcttggttgtcttgtgaaggattattttcctgagcctgtgactgtgtcatggaattcaggggctctgactagtggagtgcatacttttcctgctgtgctgcagagtagtggactgtatagtctgagttctgtggtgacagtgccatcatctagtctgggaacacaaacatatatttgtaatgtgaatcataaaccatctaatacaaaggttgataagagagtggagcctaaaagttgtgataagacacatacatgtccaccatgtcctgctcctgaatttgaaggtggtccaagtgtttttctgtttcctcctaagcctaaggatactcttatgatttcaaggactccagaagtgacttgtgtggtggttgatgttagtcatgaagatcctgaggttaaatttaattggtatgtggatggagttgaagtgcataatgcaaagacaaaaccaagggaagagcagtataattctacatatagggtggtttcagtgttgacagtgctgcatcaagattggctgaatggaaaggaatataaatgtaaggtttctaataaagctctgcctgctagtattgaaaagacaatttcaaaagcaaaaggacaaccaagggaaccacaggtttatacacttcctcctagtagggaagaaatgacaaagaatcaggttagtctgacatgtctggtgaaagggttttatccttctgatattgcagtggaatgggagtcaaatgggcagcctgaaaataattataagacaactccaccagttcttgattcagatggatctttttttctgtatagtaagctgacagtggataaatctaggtggcagcaaggtaatgtgtttagttgtagtgttatgcatgaagcactgcataatcattatactcaaaagtcactgagtctgtcaccagggaaaagaaggaagaggggaagtggagagggcagaggaagtctgctaacatgtggtgatgtggaggagaatcctggacctatggacatgagggtccctgctcagctcctggggctcctgctgctctggctctcaggtgccagatgtgagattgttctgacacagtctcctggaacactgtcactgtcaccaggagagagggcaacactgtcatgtagagcaagtcagagggtgagtagtagttatctggcttggtatcagcagaaaccagggcaggcacctagattgcttatttatgatgcttcaagtagggctacagggattcctgatagattttcagggagtgggtcagggacagattttacattgacaattagtaggttggagcctgaggattttgctgtgtattattgtcagcagtatggatctttgccttggacatttggtcaggggacaaaagtggagattaagaggacagtggcagctccatctgtgtttatttttcctcctagtgatgagcagcttaaatctgggacagcttcagtggtgtgtttgcttaataatttttatccaagggaggcaaaggtgcagtggaaggttgataatgcattgcagagtggaaattctcaggagagtgtgacagagcaggattctaaagattcaacatattctctgtctagtacactgactctgtctaaggctgattatgaaaagcataaggtgtatgcatgtgaggttacacatcaagggctgtcttctcctgtgacaaaatcatttaatagaggagaatgttgaacctgagctagc

SEQ ID NO: 22 - DNA, 3969 bp, Artificial Sequence
  Description of Artificial Sequence Synthetic polynucleotide
gcggccgctcgagatctgcgatctaagtaagcttggcattccggtactgttggtaaagccaccatgtgtcaccagcagttggtcatctcttggttttccctggtttttctggcatctcccctggtggccatctgggaactgaagaaagatgtttatgtggtggaattggattggtatcctgatgcccctggagaaatggtggtcctcacctgtgacacccctgaagaagatggtatcacctggaccttggaccagagcagtgaggtcctgggctctggcaaaaccctgaccatccaagtcaaagagtttggagatgctggccagtacacctgtcacaaaggaggggaggttctgagccattccctcctgctgcttcacaaaaaggaagatggaatttggtccactgatattctgaaggaccagaaagaacccaaaaataagacctttctgagatgtgaggccaagaattattctggaagattcacctgctggtggctgaccacaatcagtactgatttgacattcagtgtcaaaagcagcagaggctcttctgacccccaaggggtgacctgtggagctgctacactctctgcagagagagtcagaggggacaacaaggagtatgagtactcagtggagtgccaggaggacagtgcctgcccagctgctgaggagagtctgcccattgaggtcatggtggatgctgttcacaagctcaagtatgaaaactacaccagcagcttcttcatcagggacatcatcaaacctgacccacccaagaacttgcagctgaagccactgaagaattctaggcaggtggaggtcagctgggagtaccctgacacctggagtactccacattcctacttctccctgacattctgtgttcaggtccagggcaagagcaagagagaaaagaaagatagagtcttcacagacaagacctcagccacagtcatctgcaggaaaaatgccagcattagtgtgagggcccaggacagatactatagctcatcttggagtgaatgggcatctgtgccctgcagtggtggaggtggaagtggaggtggtggatctgggggtggaggcagcagaaacctccctgtggccactccagacccaggaatgttcccatgccttcaccactcccaaaacctgctgagggctgtcagcaacatgctccagaaggccagacaaactctggaattttacccttgcacttctgaagagattgatcatgaagatatcacaaaagataaaaccagcacagtggaggcctgtctgccattggaactcaccaagaatgagagttgcctgaattccagagagacctctttcatcactaatgggagttgcctggcctccagaaagacctcttttatgatggccctgtgccttagtagtatttatgaagacttgaagatgtaccaggtggagttcaagaccatgaatgcaaagcttctgatggaccctaagaggcagatctttctggatcaaaacatgctggcagttattgatgagctgatgcaggccctgaatttcaacagtgagactgtgccacaaaaatcctcccttgaagaacctgatttttataaaactaaaatcaagctctgcatccttcttcatgctttcagaattagggcagtgactattgacagagtgatgagctatctgaatgcttccaggagaaagagaggatcctctggaagtggagctactaacttcagcctgctgaagcaggctggagatgtggaggagaaccctggacctatggagtttgggctgagctgggttttccttgttgctcttttaagaggtgtccagtgtgaggtgcagcttgtggaatctgggggggggctggtgcagcctggtggtagtctgagactgtcatgtgctgctagtgggtttacattttcagattcttggattcattgggtgagacaggcacctgggaaaggtctggagtgggtggcatggatttcaccttatgggggatctacatattatgctgatagtgtgaaggggaggtttacaatttctgctgatacatctaagaatacagcttatcttcagatgaattctctgagagctgaagatactgcagtgtattattgtgctaggaggcattggcctggagggtttgattattggggtcaaggtacactggtgactgttagtagtgctagtactaaaggtccttctgtgtttccactggcaccaagttcaaagagtacatcaggagggactgcagctctgggttgtttggtgaaagattattttcctgaacctgtgacagtttcatggaattctggagcactgacttctggagtgcatacatttcctgctgtgctgcagtctagtgggttgtattcattgtcaagtgtggttacagtgccttcaagttctctgggtacacagacttatatttgtaatgtgaatcataagccaagtaatacaaaagtggataagaaagttgagcctaaatcatgtgataaaactcatacttgtccaccttgtcctgctccagagctgttgggtgggcctagtgtttttctttttccaccaaagccaaaagatactttgatgatttcaaggacaccagaagtgacatgtgtggttgttgatgtttctcatgaagatcctgaggtgaagtttaattggtatgttgatggggttgaggtgcataatgctaagacaaaacctagggaggaacagtatgcttctacatatagagttgtgtcagtgttgacagtgctgcatcaagattggcttaatgggaaagaatataagtgtaaggtttcaaataaggcattgccagctccaattgaaaagacaatttctaaggctaagggtcagcctagggagccacaggtgtatactctgccaccttcaagagaggaaatgactaagaatcaggtgtcattgacatgtttggtgaaaggattttatccttcagatattgctgtggaatgggaatctaatggacaaccagagaataattataaaactactcctcctgtgctggatagtgatggaagtttttttctgtattctaaacttactgttgataaaagtagatggcagcaaggtaatgttttttcttgttctgtgatgcatgaagctcttcataatcattatactcagaagagtctgagtctgtctcctggaaagagaaggaagaggggaagtggagagggcagaggaagtctgctaacatgtggtgatgtggaggagaatcctggacctatggacatgagggtccctgctcagctcctggggctcctgctgctctggctctcaggtgccagatgtgatattcagatgacacagagtccaagttcactgtcagcttctgttggtgatagagttactattacatgtagagcttctcaggatgtgagtactgcagtggcttggtatcagcagaagccagggaaggctccaaagctgctgatttattcagcatcatttctgtattcaggggtgccatcaagattttcaggttctggaagtggaacagattttactctgactatttcatctctgcaaccagaagattttgcaacatattattgtcagcagtatctgtatcatccagcaacatttggtcagggtactaaagtggaaattaaaaggacagtggcagcaccatcagtttttatttttccacctagtgatgaacagctgaaaagtgggacagcttcagtggtgtgtctgcttaataatttttatcctagagaagcaaaagtgcagtggaaggtggataatgcactgcaaagtgggaattcacaggaatcagtgacagagcaagattctaaggattctacatatagtctgtcttctacattgactctgtctaaggcagattatgaaaagcataaagtttatgcatgtgaggttactcatcagggattgtcatcacctgttactaaaagttttaataggggtgagtgttgaacctgagctagc

SEQ ID NO: 23 - DNA, 2237 bp, Artificial Sequence
  Description of Artificial Sequence Synthetic polynucleotide
aagcttggcattccggtactgttggtaaagccaccatgatggtgttaagtcttctgtacctgttgacagcccttcctggtatcctgtcagaggtgcagctgcaggagtcaggaccaggcctggtgaaaccttctcagagtctgtccctgacttgttctgtcactgggtattcaattacatcttcatatagatggaactggatcaggaagtttccagggaataggctggagtggatggggtacataaattcagctggtatttctaattacaatccatctctgaagagaagaatctccatcacaagagacacatccaaaaaccagttctttctgcaggttaattctgtgactactgaggatgctgccacatattactgtgcaagaagtgataatatggggacaacaccttttacttattggggtcaagggacattggtgactgtgagttctgcatcaacaacagcaccatctgtctatccactggcccctgtgtgtggagatacaactggctcctcagtgactctgggatgcctggtcaagggttatttccctgagccagtgaccttgacctggaactctggctccctgtccagtggtgtgcacaccttcccagctgtcctgcagtctgacctctacaccctcagcagctcagtgactgtaacctctagcacctggcccagccagtccatcacctgcaatgtggcccacccagcaagcagcaccaaggtggacaagaaaattgagcccagagggcccacaatcaagccctgtcctccatgcaaatgcccagcacctaatgcagctggtggaccatctgtcttcatcttccctccaaagatcaaggatgtactcatgatctccctgagccccatagtcacatgtgtggtggtggatgtgagtgaggatgacccagatgtccagatcagctggtttgtgaacaatgtggaagtacacacagctcagacacaaacccatagagaggattacaacagtactctcagggtggtcagtgccctccccatccagcaccaggactggatgagtggcaaggagttcaaatgcaaggtcaacaacaaagacctgggggcacccattgagagaaccatctcaaaacccaaagggtcagtaagagctccacaggtatatgtcttgcctccaccagaagaagagatgactaagaaacaggtcactctgacctgcatggtcacagacttcatgcctgaagacatttatgtggagtggaccaacaatgggaaaacagagctgaactacaagaacactgaaccagtcctggactctgatggttcttacttcatgtacagcaagctgagagtggaaaagaagaactgggtggaaagaaatagctactcctgttcagtggtccatgagggtctgcacaatcaccacacaactaagagcttctctaggactccaagaaggaagaggggaagtggagagggcagaggaagtctgctaacatgtggtgatgtggaggagaatcctggacctatgaggtgcctagctgagttcctggggctgcttgtgctctggattcctggagccattggggatattgtgatgactcagggtactctgcctaatcctgtgccaagtggggagtctgtgtctattacatgtaggagttcaaagagtcttctttattcagatggaaaaacatatctgaattggtatctgcagagacctgggcagagtcctcagctgctgatttattggatgtctactagggcatctggggtgtctgatagattttctggtagtggtagtggtacagattttacattgaagatttctggggtggaggctgaagatgtgggtatttattattgtcagcaaggtctggagtttccaacatttgggggaggtactaagctggagctgaagagaactgatgctgcaccaactgtatccatcttcccaccatccagtgagcagctgacatctggaggtgcctcagttgtgtgcttcctgaacaacttctaccccaaagacatcaatgtcaagtggaagattgatggcagtgaaagacaaaatggggtcctgaacagttggactgatcaggacagcaaagacagcacctacagcatgagcagcaccctcaccctgaccaaggatgagtatgaaagacataacagctatacctgtgaggccactcacaagacatcaacttcacccattgtcaagagcttcaacaggaatgagtgttgaacctgagctagc

SEQ ID NO: 24 - protein, 8 aa, Unknown
  Description of Unknown Picornavirus 2A ribosome skipping sequence
  MOD_RES (2)..(2): Val or Ile
  MOD_RES (4)..(4): Any amino acid
DXEXNPGP

SEQ ID NO: 25 - protein, 4 aa, Unknown
  Description of Unknown Furin cleavage site
RRKR